(19) United States
(12) Patent Application Publication
(10) Pub. No.: US 2009/0100545 A1
(43) Pub. Date: Apr. 16, 2009

(54) Means and Methods to Modulate Flavonoid Biosynthesis in Plants and Plant Cells

(75) Inventors: Frank Van Breusegem, Brakel (BE); Sandy Vanderauwera, Affligem (BE)

Correspondence Address: TRASK BRITT, P.O. BOX 2550, SALT LAKE CITY, UT, 84110, US

(73) Assignees: VIB BZW, Zwijnaarde (BE); Universiteit Gent, Gent (BE)

(21) Appl. No.: 11/920,395

(22) PCT Filed: Jun. 6, 2006
(86) PCT No.: PCT/EP2006/062939

§ 371 Date: Oct. 29, 2008

(30) Foreign Application Priority Data
Jun. 3, 2005 (EP) 05104855.1

(51) Int. Cl. A01H 1/00; C12N 15/82; C12N 15/63; A01H 5/00; C12N 5/10

(52) U.S. Cl. 800/282; 435/455; 435/320.1; 800/298; 435/419

(57) ABSTRACT

The present invention provides a method for increasing the flavonoid content of plants and plant cells wherein said method comprises increasing the activity of genes implicated in the flavonoid biosynthesis pathway. The invention further relates to recombinant plant and plant cells obtainable by the process of the invention and to flavonoids made there from.

### FIELD OF THE INVENTION

The present invention provides a method for increasing the flavonoid content of plants and plant cells wherein said method comprises increasing the activity of genes implicated in the flavonoid biosynthesis pathway. The invention further relates to recombinant plant and plant cells obtainable by the process of the invention and to flavonoids made there from.

### BACKGROUND OF THE INVENTION

Flavonoids are found to be ubiquitous in all vascular plants, in various parts like flowers, fruits, vegetables and seeds. These secondary metabolites form a large family of low molecular weight polyphenolic compounds and may be found under five separate headings: 1) the anthocyanins and anthochlors, which are red-to-blue and yellow flower pigments, respectively; 2) the minor flavonoids, which include flavanones, dihydro-flavonols and dihydrochalcones; 3) the flavones and flavonols, the most widely occurring and structurally variable flavonoids; 4) the isoflavonoids, a distinctive class found mainly in one plant family, the Leguminosae; and 5) the tannins, which are characterised by their affinity to bind with protein. Among the tannins are both flavonoids (the proanthocyanidins or flavolans) and simpler phenolics based on gallic acid (the gallo- and ellagi-tannins). More than 4000 flavonoids have been described, most are conjugated to sugar molecules and are commonly located in the upper epidermal layers of leaves. Reports in the prior art show that there is increasing evidence that flavonoids are potentially health-protecting components in the human diet. Indeed, several epidemiological studies suggest a direct relationship between cardioprotection and increased consumption of flavonoids, in particular flavonols of the quercetin and kaempferol type, from dietary sources such as onion, apples and tea. Flavonoids have also been reported to exhibit a wide range of biological activities in vitro including anti-inflammatory, anti-allergic and vasodilatory activity. Such activity has been attributed in part to their ability to act as antioxidants, capable of scavenging free radicals and preventing free radical production. Within this group of compounds, those having the most potent antioxidant activity are the flavonols. In addition, flavonoids can also inhibit the activity of key processes such as lipid peroxidation, platelet aggregation and capillary permeability. Flavanones and their glycosides are also considered important determinants of taste. For example, in contrast to many other fruit, the genus Citrus is characterised by a substantial accumulation of flavanone glycosides. It is noteworthy that in grapefruit the sour taste results mainly from the accumulation of the bitter flavanone glycoside, naringin. Another issue is that certain flavonoids have the ability to inhibit phytopathogens in several plant species. Flavonoid levels can also be manipulated in order to select particular flower colours and patterns. Moreover, increased amounts of condensed tannins in certain forage crops are useful for decreasing bloat in cattle, improving ruminal protein bypass, reducing intestinal parasites, and reducing sileage degradation by proteolysis. From the above it is clear that it would be desirable to produce plants and plant cell cultures which intrinsically posses, elevated levels of flavonoids. Health protecting compounds can for example be produced in plant cell cultures and isolated in pure compounds by extraction and purification. Although it is clear that the flavonoid biosynthetic pathway has been widely studied in a number of different plant species there are still many key genes unknown. The present invention has identified a transcriptional regulon of 69 genes, which are involved in the synthesis of flavonoids, more particularly anthocyanins. These genes can be used to modulate the levels flavonoids of in plants and plant cells.

### AIMS AND DETAILED DESCRIPTION OF THE INVENTION

The flavonoids comprise an astonishingly diverse and valuable group of more than 4500 known compounds. Among their subclasses are the anthocyanins (pigments), proanthocyanidins or condensed tannins (feeding deterrents and wood protectants), and isoflavonoids (defensive products and signaling molecules). The present invention has identified a transcriptional regulon of 69 genes, which can be used to modulate the production of flavonoids in plants and plant cells. Accordingly the invention provides in a first embodiment the use of polynucleotides consisting from the list SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 and/or 69 or fragments or homologues thereof to modulate the biosynthesis of flavonoids in plants or plant cells.

In yet another embodiment said polynucleotides consisting from the list SEQ ID NO: 1-69 are used to modulate the biosynthesis of anthocyanins.

In yet another embodiment the invention provides a recombinant DNA vector comprising at least one of the polynucleotide sequences selected from SEQ ID NO: 1-69.

In yet another embodiment the invention provides a transgenic plant of plant cell that is transformed with a recombinant DNA vector comprising at least one of the polynucleotide sequences selected from SEQ ID NO: 1-69.

As used herein, the word “polynucleotide” may be interpreted to mean the DNA and cDNA sequence as detailed by Yoshikai et al. (1990) Gene 87:257, with or without a promoter DNA sequence as described by Salbaum et al. (1988) EMBO J. 7(9):2807.

As used herein, “fragment” refers to a polypeptide or polynucleotide of at least about 9 amino acids or 27 base pairs, typically 50 to 75, or more amino acids or base pairs, wherein the polypeptide contains an amino acid core sequence. If desired, the fragment may be fused at either terminus to additional amino acids or base pairs, which may number from 1 to 20, typically 50 to 100, but up to 250 to 500 or more. A “functional fragment” means a polypeptide fragment possessing the biological property able to modulate the production of at least one flavonoid in an organism or cell derived thereof. In a particular embodiment said functional fragment is able to modulate the production of at least one flavonoid in a plant or plant cell derived thereof. The term ‘production’ includes intracellular production and secretion into the medium. The term ‘modulates or modulation’ refers to an increase or a decrease. Often an increase of at least one flavonoid is desired but sometimes a decrease of at least one flavonoid is wanted. Said decrease can for example refer to the decrease of an undesired intermediate product of at least one flavonoid. With an increase in the production of one or more metabolites it is understood that said production may be enhanced by at least 20%, 30%, 40%, 50%, 60%, 70%, 80%, 90% or at least 100% relative to the untransformed plant or plant cell which was used to transform with an expression vector comprising an expression cassette further comprising at least one polynucleotide or homologue or variant or fragment thereof of the invention. Conversely, a decrease in the production of the level of one or more flavonoids may be decreased by at least 20%, 30%, 40%, 50%, 60%, 70%, 80%, 90% or at least 100% relative to the untransformed plant or plant cell which was used to transform with an expression vector comprising an expression cassette further comprising at least one polynucleotide or homologue or variant or fragment thereof of the invention. The terms ‘identical’ or percent ‘identity’ in the context of two or more nucleic adds or polypeptide sequences, refer to two or more sequences or subsequences that are the same or have a specified percentage of amino add residues or nucleotides that are the same (i.e. 70% identity over a specified region), when compared and aligned for maximum correspondence over a comparison window, or designated region as measured using sequence comparison algorithms or by manual alignment and visual inspection. Preferably, the identity exists over a region that is at least about 25 amino acids or nucleotides in length, or more preferably over a region that is 50-100 amino acids or nucleotides or even more in length. Examples of useful algorithms are PILEUP (Higgins & Sharp, CABIOS 5:151 (1989), BLAST and BLAST 2.0 (Altschul et al. J. Mol. Biol. 215: 403 (1990). Software for performing BLAST analyses is publicly available through the National Center for Biotechnology Information (http://www/ncbi.nlm.nih.gov/). In the present invention the term ‘homologue’ also refers to ‘identity’. For example a homologue of SEQ ID NO: 1-69 has at least 60% identity to one of these sequences. According to still further features in the described preferred embodiments the polynucleotide fragment encodes a polypeptide able to modulate the flavonoid biosynthesis, which may therefore be allelic, species and/or induced variant of the amino acid sequence set forth in SEQ ID NO: 1-69. It is understood that any such variant may also be considered a homologue.

The present invention accordingly provides in another embodiment a method for modulating the production of at least one flavonoid in plant or plant cells, by transformation of said plant or plant cells with an expression vector comprising an expression cassette that further comprises at least one gene comprising a fragment, variant or homologue encoded by at least one sequence selected from SEQ ID NO: 1-69.

In another embodiment the invention provides a recombinant DNA vector comprising at least one polynucleotide sequence, homologue, fragment or variant selected from at least one of the sequences comprising SEQ ID NO: 1-69. The vector may be of any suitable type including, but not limited to, a phage, virus, plasmid, phagemid, cosmid, bacmid or even an artificial chromosome. The at least one polynucleotide sequence preferably codes for at least one polypeptide that is involved in the biosynthesis and/or regulation of synthesis of at least one flavonoid (e.g. a transcription factor, a repressor, an enzyme that regulates a feed-back loop, a transporter, a chaperone). The term “recombinant DNA vector” as used herein refers to DNA sequences containing a desired coding sequence and appropriate DNA sequences necessary for the expression of the operably linked coding polynucleotide sequence in a particular host organism (e.g. plant cell). Plant cells are known to utilize promoters, polyadenylation signals and enhancers.

In yet another embodiment the invention provides a transgenic plant or derived cell thereof transformed with said recombinant DNA vector.

A recombinant DNA vector comprises at least one “Expression cassette”. Expression cassettes are generally DNA constructs preferably including (5′ to 3′ in the direction of transcription): a promoter region, a polynucleotide sequence, homologue, variant or fragment thereof of the present invention operatively linked with the transcription initiation region, and a termination sequence including a stop signal for RNA polymerase and a polyadenylation signal. It is understood that all of these regions should be capable of operating in biological cells, such as plant cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The polynucleotide sequence, homologue, variant or fragment thereof of the invention may be expressed in for example a plant cell under the control of a promoter that directs constitutive expression or regulated expression. Regulated expression comprises temporally or spatially regulated expression and any other form of inducible or repressible expression. Temporally means that the expression is induced at a certain time point, for instance, when a certain growth rate of the plant cell culture is obtained (e.g. the promoter is induced only in the stationary phase or at a certain stage of development). Spatially means that the promoter is only active in specific organs, tissues, or cells (e.g. only in roots, leaves, epidermis, guard cells or the like). Other examples of regulated expression comprise promoters whose activity is induced or repressed by adding chemical or physical stimuli to the plant cell. In a preferred embodiment the expression is under control of environmental, hormonal, chemical, and/or developmental signals. Such promoters for plant cells include promoters that are regulated by (1) heat, (2) light, (3) hormones, such as abscisic add and methyl jasmonate (4) wounding or (5) chemicals such as salicylic acid, chitosans or metals. Indeed, it is well known that the expression of secondary metabolites (such as flavonoids) can be boosted by the addition of for example specific chemicals, jasmonate and elicitors. In a particular embodiment the co-expression of several (more than one) polynucleotide sequence or homologue or variant or fragment thereof, in combination with the induction of secondary metabolite synthesis is beneficial for an optimal and enhanced production of flavonoids. Alternatively, the at least one polynucleotide sequence, homologue, variant or fragment thereof is placed under the control of a constitutive promoter. A constitutive promoter directs expression in a wide range of cells under a wide range of conditions. Examples of constitutive plant promoters useful for expressing heterologous polypeptides in plant cells include, but are not limited to, the cauliflower mosaic virus (CaMV) 35S promoter, which confers constitutive, high-level expression in most plant tissues including monocots; the nopaline synthase promoter and the octopine synthase promoter. The expression cassette is usually provided in a DNA or RNA construct which is typically called an “expression vector” which is any genetic element, e.g., a plasmid, a chromosome, a virus, behaving either as an autonomous unit of polynucleotide replication within a cell (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages, cosmids, plant viruses and artificial chromosomes. The expression cassette may be provided in a DNA construct which also has at least one replication system. In addition to the replication system, there will frequently be at least one marker present, which may be useful in one or more hosts, or different markers for individual hosts. The markers may a) code for protection against a biocide, such as antibiotics, toxins, heavy metals, certain sugars or the like; b) provide complementation, by imparting prototrophy to an auxotrophic host: or c) provide a visible phenotype through the production of a novel compound in the plant. Exemplary genes, which may be employed, include neomycin phosphotransferase (NPTII), hygromycin phosphotransferase (HPT), chloramphenicol acetyltransferase (CAT), nitrilase, and the gentamicin resistance gene. For plant host selection, non-limiting examples of suitable markers are β-glucuronidase, providing indigo production, luciferase, providing visible light production, Green Fluorescent Protein and variants thereof, NPTII, providing kanamycin resistance or G418 resistance, HPT, providing hygromycin resistance, and the mutated aroA gene, providing glyphosate resistance.

The term “promoter activity” refers to the extent of transcription of a polynucleotide sequence, homologue, variant or fragment thereof that is operably linked to the promoter whose promoter activity is being measured. The promoter activity may be measured directly by measuring the amount of RNA transcript produced, for example by Northern blot or indirectly by measuring the product coded for by the RNA transcript, such as when a reporter gene is linked to the promoter. The term “operably linked” refers to linkage of a DNA segment to another DNA segment in such a way as to allow the segments to function in their intended manners. A DNA sequence encoding a gene product is operably linked to a regulatory sequence when it is ligated to the regulatory sequence, such as, for example a promoter, in a manner, which allows modulation of transcription of the DNA sequence, directly or indirectly. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art.

In a particular embodiment the polynucleotides or homologues or variants or fragments thereof of the present invention can be introduced in plants or plant cells that are different from Arabidopsis and said polynucleotides can be used for the modulation of flavonoid synthesis in plants or plant cells.

The term “heterologous DNA” and or “heterologous RNA” refers to DNA or RNA that does not occur naturally as part of the genome or DNA or RNA sequence in which it is present, or that is found in a cell or location in the genome or DNA or RNA sequence that differs from that which is found in nature. Heterologous DNA and RNA (in contrast to homologous DNA and RNA) are not endogenous to the cell into which it is introduced, but has been obtained from another cell or synthetically or recombinantly produced. An example is a gene isolated from one plant species operably linked to a promoter isolated from another plant species. Generally, though not necessarily, such DNA encodes RNA and proteins that are not normally produced by the cell in which the DNA is transcribed or expressed. Similarly exogenous RNA encodes for proteins not normally expressed in the cell in which the exogenous RNA is present. Heterologous DNA or RNA may also refer to as foreign DNA or RNA. Any DNA or RNA that one of skill in the art would recognize as heterologous or foreign to the cell in which it is expressed is herein encompassed by the term heterologous DNA or heterologous RNA. Examples of heterologous DNA include, but are not limited to, DNA that encodes proteins, polypeptides, receptors, reporter genes, transcriptional and translational regulatory sequences, selectable or traceable marker proteins, such as a protein that confers drug resistance, RNA including mRNA and antisense RNA and ribozymes.

Accordingly, the invention provides in a further aspect a gene construct in the form of an expression cassette comprising as operably linked components in the 5′-3′ direction of transcription, one or more units each comprising a suitable promoter in a plant cell, a plurality of nucleotide sequences selected from the group consisting of sequences SEQ ID NO: 1-69 for flavonoid biosynthesis and a suitable transcriptional and translational termination regulatory region.

The promoter and termination regulatory regions will be functional in the host plant cell and may be heterologous or homologous to the plant cell and the gene. Suitable promoters, which may be used, are described above.

The termination regulatory region may be derived from the 3′ region of the gene from which the promoter was obtained or from another gene. Suitable termination regions, which may be used, are well known in the art and include Agrobacterium tumefaciens nopaline synthase terminator (Tnos), Agrobacterium tumefaciens mannopine synthase terminator (Tmas), the rubisco small subunit terminator (TrbcS) and the Ca 35S terminator (T35S).

The present invention can be practiced with any plant variety for which cells of the plant can be transformed with an expression cassette of the current invention and for which transformed cells can be cultured in vitro. Suspension culture, callus culture, hairy root culture, shoot culture or other conventional plant cell culture methods may be used (as described in: Drugs of Natural Origin, G. Samuelsson, 1999, ISBN 9186274813).

By “plant cells” it is understood any cell which is derived from a plant and can be subsequently propagated as callus, plant cells in suspension, organized tissue and organs (e.g. hairy roots). Tissue cultures derived from the plant tissue of interest can be established. Methods for establishing and maintaining plant tissue cultures are well known in the art (see, e.g. Trigiano R. N. and Gray D. J. (1999), “Plant Tissue Culture Concepts and Laboratory Exercises”, ISBN: 0-8493-2029-1; Herman E. B. (2000), “Regeneration and Micropropagation: Techniques, Systems and Media 1997-1999”, Agricell Report). Typically, the plant material is surface-sterilized prior to introducing it to the culture medium. Any conventional sterilization technique, such as chlorinated bleach treatment can be used. In addition, antimicrobial agents may be included in the growth medium. Under appropriate conditions plant tissue cells form callus tissue, which may be grown either as solid tissue on solidified medium or as a cell suspension in a liquid medium.

A number of suitable culture media for callus induction and subsequent growth on aqueous or solidified media are known. Exemplary media include standard growth media, many of which are commercially available (e.g., Sigma Chemical Co., St. Louis, Mo.). Examples include Schenk-Hildebrandt (SH) medium, Linsmaier-Skoog (LS) medium, Murashige and Skoog (MS) medium, Gamborg's B5 medium, Nitsch & Nitsch medium, White's medium, and other variations and supplements well known to those of skill in the art (see, e.g., Plant Cell Culture, Dixon, ed. IRL Press, Ltd. Oxford (1985) and George et al., Plant Culture Media, Vol 1, Formulations and Uses Exegetics Ltd. Wilts, UK, (1987)). For the growth of conifer cells, particularly suitable media include 1/2 MS, 1/2 L.P., DCR, Woody Plant Medium (WPM), Gamborg's B5 and its modifications, DV (Durzan and Ventimiglia, In Vitro Cell Dev. Biol. 30:219-227 (1994)), SH, and White's medium.

In a particular embodiment the current invention can be combined with other known methods to enhance the production and/or the secretion of flavonoids in plant cell cultures such as (1) by improvement of the plant cell culture conditions, (2) by the transformation of the plant cells with a transcription factor capable to induce genes involved in the pathway of flavonoid formation, (3) by the addition of specific elicitors to the plant cell culture, and 4) by the induction of organogenesis.

The term “plant” as used herein refers to vascular plants (e.g. gymnosperms and angiosperms). The method comprises transforming a plant cell with an expression cassette of the present invention and regenerating such plant cell into a transgenic plant. Such plants can be propagated vegetatively or reproductively. The transforming step may be carried out by any suitable means, including by Agrobacterium-mediated transformation and non-Agrobacterium-mediated transformation, as discussed in detail below. Plants can be regenerated from the transformed cell (or cells) by techniques known to those skilled in the art. Where chimeric plants are produced by the process, plants in which all cells are transformed may be regenerated from chimeric plants having transformed germ cells, as is known in the art. Methods that can be used to transform plant cells or tissue with expression vectors of the present invention include both Agrobacterium and non-Agrobacterium vectors. Agrobacterium-mediated gene transfer exploits the natural ability of Agrobacterium tumefaciens to transfer DNA into plant chromosomes and is described in detail in Gheysen, G., Angenon, G. and Van Montagu, M. 1998. Agrobacterium-mediated plant transformation: a scientifically intriguing story with significant applications. In K. Lindsey (Ed.), Transgenic Plant Research. Harwood Academic Publishers, Amsterdam, pp. 1-33 and in Stafford, H. A. (2000) Botanical Review 66: 99-118. A second group of transformation methods is the non-Agrobacterium mediated transformation and these methods are known as direct gene transfer methods. An overview is brought by Barcelo, P. and Lazzeri, P. A. (1998) Direct gene transfer: chemical, electrical and physical methods. In K. Lindsey (Ed.), Transgenic Plant Research, Harwood Academic Publishers, Amsterdam, pp. 35-55. Hairy root cultures can be obtained by transformation with virulent strains of Agrobacterium rhizogenes, and they can produce high contents of secondary metabolites characteristic to the mother plant. Protocols used for establishing of hairy root cultures vary, as well as the susceptibility of plant species to infection by Agrobacterium (Toivounen L. (1993) Biotechnol. Prog. 9, 12; Vanhala L. et al. (1995) Plant Cell Rep. 14, 236). It is known that the Agrobacterium strain used for transformation has a great influence on root morphology and the degree of secondary metabolite accumulation in hairy root cultures. It is possible that by systematic done selection e.g. via protoplasts, to find high yielding, stable, and from single cell derived-hairy root clones. This is possible because the hairy root cultures possess a great somaclonal variation. Another possibility of transformation is the use of viral vectors (Turpen TH (1999) Philos Trans R Soc Lond B Biol Sci 354(1383): 665-73).

Any plant tissue or plant cells capable of subsequent clonal propagation, whether by organogenesis or embryogenesis, may be transformed with an expression vector of the present invention. The term ‘organogenesis’ means a process by which shoots and roots are developed sequentially from meristematic centers; the term ‘embryogenesis’ means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include protoplasts, leaf disks, pollen, embryos, cotyledons, hypocotyls, megagametophytes, callus tissue, existing meristematic tissue (e.g. apical meristems, axillary buds, and root meristems), and induced meristem tissue (e.g., cotyledon meristem and hypocotyls meristem).

These plants may include, but not limited to, plants or plant cells of agronomically important crops, such as plants from the Pisum family such as peas, family of Brassicae, such as green cabbage, Brussel sprouts, cauliflower, the family of Phaseolus such as barlotti beans, green beans, kidney beans, the family of Spinacea such as spinach, the family of Solanaceae such as potato and tomato, the family of Daucus, such as carrots, family of Capsicum such as green and red pepper, and the family of Ribesiaceae such as strawberries, blackberries, raspberries, black currant and edible grasses from the family of Gramineae such as maize, and citrus fruit for example from the family of Rutaceae such as lemon, orange, tangerine, or from the apple family. Also preferred are oil producing plants such as sunflower, soybean and rape. Also preferred are plants which can form the basis of an infusion such as black tea leaves, green tea leaves, jasmine tea leaves. It is also understood that the invention may be applied to plants that produce valuable compounds. Examples of such plants include, but not limited to, Papaver spp., Rauwolfia spp., Taxus spp., Cinchona spp., Eschscholtzia californica, Camptotheca acuminata, Hyoscyamus spp., Berberis spp., Coptis spp., Datura spp., Atropa spp., Thalictrum spp., Peganum spp.

It may well be that increase in flavonoid content observed in plants modified according to the invention comprises an increase in a plurality of different flavonoid types depending on the nature of the plant tissue in which modified gene expression is occurring.

In yet another embodiment suitable expression cassettes comprising the nucleotide sequences of the present invention can be used for transformation into other species (different from Arabidopsis). This transformation into other species or genera can be carried out randomly or can be carried out with strategically chosen nucleotide sequences. The random combination of genetic material from one or more species of organisms can lead to the generation of novel metabolic pathways (for example through the interaction with metabolic pathways resident in the host organism or alternatively silent metabolic pathways can be unmasked) and eventually lead to the production of novel classes of compounds. This novel or reconstituted metabolic pathways can have utility in the commercial production of novel, valuable flavonoids.

Various assays within the knowledge of the person skilled in the art may be used to determine whether the plant cell shows an increase in gene expression, for example, Northern blotting or quantitative reverse transcriptase PCR (RT-PCR). Whole transgenic plants may be regenerated from the transformed cell by conventional methods. Such transgenic plants having improved flavonoid levels may be propagated and crossed to produce homozygous lines. Such plants produce seeds containing the genes for the introduced trait and can be grown to produce plants that will produce the selected phenotype.

The recombinant DNA and molecular cloning techniques applied in the below examples are all standard methods well known in the art and are e.g. described by Sambrook et al. (1989) Molecular cloning: A laboratory manual, second edition, Cold Spring Harbor Laboratory Press. Methods for tobacco cell culture and manipulation applied in the below examples are methods described in or derived from methods described in Nagata et al. (1992) Int. Rev. Cytol. 132, 1.

### EXAMPLES

### 1. Identification of 69 Genes Involved in Flavonoid Biosynthesis

Genome-wide analysis of photorespiratory hydrogen peroxide regulated gene expression in Arabidopsis reveals a high light induced transcriptional regulon involved in anthocyanin biosynthesis.

By using ATH1 Affymetrix microarrays, expression profiles were compared between control and catalase-deficient Arabidopsis thaliana plants. Reduced catalase levels already provoked differences in nuclear gene expression under ambient growth conditions and these effects are amplified by high light exposure in a sun simulator for 3 and 8 h. Genome-wide expression analysis allowed the characterization of complete pathways and functional categories during H₂O₂ stress. In addition by analyzing transcriptome data sets obtained from a combination of different perturbations it becomes possible to identify more robustly co-regulated genes over a wide range of stresses, which are to be part of the same regulon and, therefore, to be considered as “brothers in arms” within the studied biological process. From such a “guilt by assocation” analysis the function of hitherto unknown genes can be predicted with more certainty. Through the analysis of transcriptomic changes provoked by photorespiratory H₂O₂, a transcriptional regulon of genes associated with anthocyanin biosynthesis was identified. In addition to the genes known to be involved in anthocyanin biosynthesis, several unknown genes that can be put forward as potential candidates for a function within the production of anthocyanins in leaves.

The 1495 differentially expressed genes with CV>2 were subjected to hierarchical average linkage clustering. Different prominent clusters of transcriptional changes stand out clearly: cluster A (484 genes) represents mainly genotype-independent HL-repressed genes; cluster B groups 437 genes that are exclusively induced by HL in the CAT2HP1 plants; cluster C contains 111 genes that are repressed in the CAT2HP1 plants and cluster D (463 genes) comprises mainly (genotype independent) HL-induced genes.

As an alternative for a CV analysis, genes were classified according to their fold change in expression. Therefore, the threshold for positive response was set at threefold change in expression. The expression of 906 genes was affected by HL itself. Of the 906 exclusively HL differentially regulated genes, 379 were upregulated and 527 were downregulated. Screening for differentially expressed genes in response to photorespiratory H₂O₂, revealed 349 and 88H₂O₂-induced or H₂O₂-repressed genes, respectively. In our analysis, HL drives after 3 h the upregulation of nearly 380 genes in control plants. When assessing the expression profiles of these genes in both HL-exposed CAT2HP1 and control plants, a clear subcluster could be recognized in which the induction by HL was significantly delayed in the CAT2HP1 plants. Whereas in control plants transcripts levels increased rapidly within 3 h of HL, they only reached their highest expression levels after 8 h in the CAT2HP1 plants. Within this subcluster, genes known to be involved in the regulation, biosynthesis and sequestration of anthocyanins were predominantly present. To enable a more robust identification of other genes in the regulon, we selected all genes whose expression levels were at least threefold induced after 3 h or 8 h of HL stress, but had at least a 1.5-fold lower expression in the CAT2HP1 compared to control plants. The expression characteristics of 176 genes matched these criteria. To further validate the robustness of the selected genes, we assessed their expression during leaf senescence. Senescence is a well-characterized process in which anthocyanin levels are upregulated (Hoch W A et al (2001) Tree Physiol. 21, 1-8). Expression profiles of the 176 genes during the HL treatment and their behavior during senescence were clustered together and resulted unexpectedly in a major division into two prominent clusters. Cluster B (105 genes) grouped genes involved in the anthocyanin biosynthesis and regulation together with 69 genes previously not associated with anthocyanin biosynthesis and/or regulation.

### 2. Functional Analysis of the Genes

Full-length cDNAs were PCR-amplified with gene-specific primers from cDNA obtained from Col-0 Arabidopsis plants and cloned into the GateWay destination vector pB7WG2D, which is a binary vector for overexpression in plants (Karimi et al. (2002) Trends Plant Sci. 7(5):193-5). Constructs were transformed into Arabidopsis thaliana Col-0 plants through Agrobacterium-mediated floral dip transformation (Clough and Bent (1998) Plant J 16(6):735-43). Primary transformants were selected through resistance to basta resistance and were selfed. Progeny plants were assessed for transgene overexpression through RT-PCR, Northern blot analysis or Western blot analysis and segregation analysis was performed to identify lines with single T-DNA locus. Selected lines were subjected to a phenotypic analysis (visual scoring for increased coloration) and biochemical analysis (determination of anthocyanins via methanol-extraction or HPLC analysis) under ambient and high light conditions (1000 μmol m⁻² sec⁻¹).

### 3. Anthocyanin Measurements

Plants were grown on MS medium for 14 days and exposed to continuous HL irradiation (approximately 1000 μmol m⁻² sec⁻¹) for 23 h. Fresh weight was recorded for each sample, and ranged from 0.099 to 0.185 g per sample. Samples were frozen in liquid nitrogen en ground with mortar and pestle. Anthocyanins were measured according to a procedure based on the methods of Rabino and Mancinelli (1986), and Feinbaum and Ausubel (1988). Total plant pigments were extracted in 0.75 ml of 1% HCl/methanol, and 0.5 ml of distilled H₂O was added. Chlorophyll was separated from the anthocyanins by back-extraction with chloroform. The quantity of anthocyanin pigments was determined by spectrophotometric measurements of the aqueous/methanol phase. The absorbance at 530 nm minus the absorbance at 657 nm was used as a measure of anthocyanin content, and values were normalized to the fresh weight of each sample. Results are expressed as absorbance per g FW. The results of some transgenic lines are presented in Table 1.

4. Analysis of Anthocyanins from Arabidopsis Thaliana Via HPLC

### Sample Preparation

Leaves are harvested, freeze-dried and gently grinded into a rough powder. Approx. 100±10 mg of sample is weighed exactly into a test tube and extracted with 2 ml of MeOH for 1 hour using magnetic stirrer (700 rpm). The tube is centrifuged (10 min, 3000 rpm) and the supernatant is collected. Extraction procedure is repeated with 30 minutes extraction time and the supernatants are combined. The extract is then filtrated through a 0.45 μm syringe filter. Chlorophyll is removed from the extract to avoid interference in the analysis of the anthocyanins by adding water (half the volume of the extract) and petroleum ether (1:1 with the MeOH—H₂O solution) and vortexing for 5 seconds. After 15 minutes the petroleum ether fraction containing chlorophyll is removed. The petroleum ether extraction is repeated three times. The remaining extract is evaporated to dryness. The dry extract is weighed and dissolved in 1 ml of MeOH. The sample is hydrolysed to formulate the anthocyanins into aglycons. 200 μl of 37% HCl is added and the sample is held in 90° C. water bath for 60 minutes. Anthocyanin aglycons are analysed by HPLC. Cyanidin chloride is used as an external standard (concentration range from 8.4 ppm to 210 ppm).

### Detection

HPLC analysis is performed using Waters equipment combined with PDA detector and with Empower software. Reverse-phase separation is attained in room temperature using an Agilent Hypersil C-18 (5 μm, 4.6×150 mm) column. Samples of 30 μl were injected. A gradient solvent system is used with solvent A being formic acid/water (10:90 v/v), solvent B being methanol/acetonitrile/formic acid/water (10:1:10:79 v/v/v/v) and solvent C being methanol/formic acid/water (10:10:80 v/v/v). The following gradient, with a flow rate 0.9 ml/min, is used for elution: from 0 to 24 min 80-40% A and 20-60% B, from 24 to 36 min 40-20% A and 60-80% C, from 36 to 37 min 20-80% A and 80% C to 20% B followed by isocratic elution from 37 to 50 min 80% A with 20% B.

### Materials and Methods

### Plant Material, Growth Conditions and Stress Treatments

Catalase deficient (CAT2HP1) and control (PTHW) plants were obtained as described by Vandenabeele et al. (2004). Unless mentioned otherwise, the plants were grown under controlled conditions in phytotron exposure chambers, which had been specially designed for plant stress research (Thiel et al., 1996). The light regime was 12 h/12 h at 100-140 μmol m⁻² sec⁻¹, the climate adjusted to a relative humidity of 70% and 22° C. day/18° C. night temperatures. For high light (HL) treatments, six-week-old plants were transferred to a sun simulator with identical growth conditions and exposed to continuous HL irradiation (photosynthetically active radiation 400-700 nm at approximately 1600-1800 μmol m⁻² sec⁻¹). 0, 3 and 8 hours after the onset of HL stress, middle-aged leaves of 20-30 plants per line were sampled and pooled for RNA-analysis. The two biological repeat experiments were done with a temporal interval of one year.

### Microarray Analysis

In two independent experiments, RNA was isolated from 20-30 control or catalase deficient plants using TRIzol Reagent (Invitrogen, Carlsbad, Calif., USA). The concentration of total RNA was determined with a Nanodrop ND-1000 spectrophotometer, and the quality was examined with the RNA 6000 Nano Assay (Agilent Technologies, 2100 Bioanalyzer). Each of the different pools of control and CAT2HP1 plants, subjected to 0, 3 and 8 hours of HL irradiation, was hybridized to one Affymetrix chip (Genechip® Arabidopsis ATH1 Genome Array; Affymetrix, Santa Clara, Calif., USA). For each hybridization, 15 micrograms of total RNA was used. Affymetrix chip analyses were performed at the ETH-Functional Genomics Center (Zurich, Switzerland) and the VIB Microarray Facility (Leuven, Belgium), respectively. Conditions for reverse transcription, RNA labeling, hybridization and scanning were performed according to manufacturer's instructions (https://www.affymetrix.com/). Raw data were processed with the statistical algorithm of Affymetrix Microarray Suite (MAS) 5.0 as described by Liu et al. (2002). Subsequently, a per chip normalization was performed, dividing all measurements on each chip by the 50^{th} percentile value (median). To calculate the median, measurements were limited by flag values: only measurements flagged as present were used. Genes with at least four present calls over the 12 different data points were retained for further analysis. Expression values were obtained by taking the average of the normalized values of the two independent repeats. As a selection criterion for differential expression a coefficient of variation (CV) was used, which was calculated as the ratio of the standard deviation on all measurements of the time course and the (absolute value of the) average expression over the time course. Expression values of genes with a CV higher than 2 were taken for hierarchical cluster analysis, using CLUSTER and TREEVIEW software (Eisen et al., 1998), to obtain a global view of the transcriptional changes. For the in pair comparison at different time points, fold changes were calculated using the average expression value of the two independent experiments. Only fold changes with at least two present calls (i.e. detectable expression) over the four data points were used. Analyses were based on annotations compiled by TAIR (http://www.arabidopsis.org/)

### Publicly Available Affymetrix GeneChip Data

The GeneChip data were retrieved from the international AtGenExpress repository (from The Arabidopsis Functional Genomics Network—http://www.uni-frankfurt.de/fb15/botanik/mcb/AFGN/AFGNHome.html) and downloaded from TAIR (http://www.arabidopsis.org/servlets/Search?type=expr&search_action=new search). Raw data were processed with the statistical algorithm of Affymetrix MAS 5.0 (Liu et al., 2002) and we performed a per chip normalization as described above. Growth stage annotations were based on Boyes et al. (2001).

### Tables

TABLE 1
Anthocyanin measurements in transgenic Arabidopsis thaliana lines
overexpressing SEQ ID NO: 13, 15, 18, 23, 24, 30, 52, 54, 56, 63 and 68
versus the untransformed line. Values are means values from three
independent transformants. Anthocyanin values are expressed as
absorbance per gram fresh weight (abs/g Fw).

| | Anthocyanin |
|---|---|
| Transgenic line | content (abs/g Fw) |
| SEQ ID 13 | 0.11 |
| SEQ ID 15 | 0.14 |
| SEQ ID 18 | 0.21 |
| SEQ ID 23 | 0.25 |
| SEQ ID 24 | 0.12 |
| SEQ ID 30 | 0.15 |
| SEQ ID 52 | 0.17 |
| SEQ ID 54 | 0.11 |
| SEQ ID 56 | 0.13 |
| SEQ ID 63 | 0.14 |
| SEQ ID 68 | 0.11 |
| Untransformed line | 0.06 |

1. In a method of modulating biosynthesis of a flavonoid in a plant or plant cell, the method being of the type utilizing a polynucleotide in the plant or plant cell, the improvement comprising:
utilizing, as the polynucleotide, a nucleotide selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, a fragment of any thereof, a homolog of any thereof, and combinations of any thereof
so as to modulate the biosynthesis of a flavonoid in the plant or plant cell.

2. The method according to claim 1 wherein said flavonoid is a anthocyan.

3. A recombinant DNA vector comprising at least one polynucleotide sequence selected from the group consisting of SEQ ID NO: 1-69.

4. A transgenic plant that is transformed with the recombinant DNA vector according to claim 3.

5. A plant cell comprising the recombinant DNA vector of claim 3.


SEQUENCE LISTING (69 sequences)

SEQ ID NO: 1 - DNA, 2004 bp, Arabidopsis thaliana
atgtggcaaacgtggccacgtcagccaattctactagatattttttcaaatccaaatactctttccacaaccgttagatcatggtcggttcgccacccactttcaatcataaccgttaaaacattcgctagattttttctagatattttcttttctccacactattatagaaagaataaagttcttttttttgctctcttctcatttatctctccactcacaaatattttgatttgttttgtaactgtttctctttctctggagctttcttcttcttcttcaataatcgatttaggtttttcaaagctaagtgtttgtgttgtgataatgactagtagcgaggaagtagttgaagtgacggtggttaaagcacctgaagctggcggaggaaagttatcacgtcggaagattcggaagaaagacgccggtgttgatggtttggtgaagtgggagagatttctcccgaaaatcgcgcttagagttttgctcgttgaagctgatgattctactagacagattatcgctgctcttctcaggaaatgtagttacagagttgctgcagtacctgatggcttaaaagcttgggagatgctaaaaggaaagcctgaaagtgttgatttgatattaacagaggttgatctaccttcaatatctggatatgctctgctaacacttatcatggagcatgatatttgcaagaacattcctgttataatgatgtcgacacaggactcggtgaatactgtgtataagtgtatgttgaaaggtgcggctgattatcttgttaagccgttgaggaggaatgagcttagaaatctttggcagcatgtctggagaagacaaacttcacttgctcctgatagctttccatggaatgagagtgttggacagcagaaagccgagggtgcgtctgcaaacaactcgaacggaaagagagacgatcatgttgtgagtgggaatggtggtgatgcccagagctcgtgtacaagaccagagatggaaggtgagagcgcagacgtggaggttagtgcgagagacgcagtacagatggagtgcgcaaagtctcagtttaatgagacacggcttctagcaaatgagttgcagagtaagcaagcagaagccattgacttcatgggagcatcgtttagaagaactggacgacgtaacagagaagaaagtgttgctcaatacgaatctcggatagagcttgatctttctctgagaagacctaatgcttctgagaaccaatcttctggagaccggccttctcttcatccttctagtgcctcagctttcacacggtacgttcacaggccgttgcagacacaatgttcagcctccccagtggttactgatcaaagaaagaatgttgcagcaagtcaagatgataacattgtgctaatgaaccaatacaatacatctgaaccgcctccaaatgctccaagaagaaacgacaccagcttttacactggagctgactcacctggtccaccgtttagtaatcagctgaattcttggccgggacagagttcataccctacgccaacccctatcaacaatatacagttcagagatcccaacacagcttatacatctgcaatggctcctgcttcactctccccaagccctagttccgttagcccgcatgagtacagttccatgtttcacccattcaacagtaaacccgaggggttacaagaccgggattgttccatggatgtagatgagaggagatacgtctcttctgcaaccgaacatagtgcaataggcaatcacattgatcagcttattgagaagaagaacgaagatggctattcattatccgtcgggaaaattcagcaatctcttcaacgagaagccgctttaaccaaattccgaatgaagcgaaaggacagatgttatgagaaaaaggttcgttacgagagccggaagaaattagcagagcaacgaccacgaatcaaaggccaattcgttcgtcaagtccaatccacacaagctccatag

SEQ ID NO: 2 - DNA, 651 bp, Arabidopsis thaliana
atgaactcattttctgctttttctgaaatgtttggctccgattacgagtcttcggtttcctcaggcggtgattatattccgacgcttgcgagcagctgccccaagaaaccggcgggtcgtaagaagtttcgtgagactcgtcacccaatatacagaggagttcgtcggagaaactccggtaagtgggtttgtgaggttagagaaccaaacaagaaaacaaggatttggctcggaacatttcaaaccgctgagatggcagctcgagctcacgacgttgccgctttagcccttcgtggccgatcagcctgtctcaatttcgctgactcggcttggagactccgaatcccggaatcaacttgcgctaaggacatccaaaaggcggcggctgaagctgcgttggcgtttcaggatgagatgtgtgatgcgacgacggatcatggcttcgacatggaggagacgttggtggaggctatttacacggcggaacagagcgaaaatgcgttttatatgcacgatgaggcgatgtttgagatgccgagtttgttggctaatatggcagaagggatgcttttgccgcttccgtccgtacagtggaatcataatcatgaagtcgacggcgatgatgacgacgtatcgttatggagttattaa

SEQ ID NO: 3 - DNA, 540 bp, Arabidopsis thaliana
atgcaagactcttcctctcacgaatcgcaacgtaacctccggtcaccggtgccggagaaaaccggaaagagttctaagactaaaaatgagcaaaaaggtgtttctaaacaaccaaattttcgtggggtcagaatgagacaatggggaaaatgggtgtctgaaattagagaaccaagaaagaaatcaagaatatggctcggtactttctctacgccggagatggcggcgcgtgcacacgacgtggcggctttagccatcaaaggtggctctgcccaccttaatttcccggagctagcttaccatttgccgagaccggctagcgcggaccctaaagacattcaagaagccgccgccgcagcagctgccgttgactggaaagcaccggagtctccgtctagcaccgtgacgtcatctccagtcgccgacgacgctttctccgatcttcctgatcttttgcttgacgtgaatgatcacaacaaaaacgatggattctgggactcgtttccgtacgaagatcctttcttcttggaaaattactag

SEQ ID NO: 4 - DNA, 642 bp, Arabidopsis thaliana
atgaactcattttcagctttttctgaaatgtttggctccgattacgagcctcaaggcggagattattgtccgacgttggccacgagttgtccgaagaaaccggcgggccgtaagaagtttcgtgagactcgtcacccaatttacagaggagttcgtcaaagaaactccggtaagtgggtttctgaagtgagagagccaaacaagaaaaccaggatttggctcgggactttccaaaccgctgagatggcagctcgtgctcacgacgtcgctgcattagccctccgtggccgatcagcatgtctcaacttcgctgactcggcttggcggctacgaatcccggagtcaacatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaa

SEQ ID NO: 5 - DNA, 927 bp, Arabidopsis thaliana
atggcgtcgtggatgaaagcggtgctaatctctactggcgtcgtagccacggctatgcatctaaaggttattgttcctgtggctatggatttctcacaaaatccgattattttgagctctttcctcacgtggctgaaaccgccgtatctttacgtcatcactaacgtcatcatcatcgttgtcggagtttcctaccggattactactgtctccagccacgtcgacggcaaagactatgaggcttcttacagtggcgacaataagtttcagactgatcatcagcagatcgtccaagaagctcctctaaggcgacgaacggagacgaaagatgcggattttggtttcatcggcaaagttttgcagatcgttaaggagccggaggttgtgtatgaagagaaggagaggccggcgacggtagaggaggaggagaagaagtgtataattgtggtgagcaaatcggaaaatcaacctccggtggagaagcctcttgttacggctaggatcggccaaaagaaaccggtggttaagactacaccagcagaaaggaattctatgagagcgttgagagttgcgaaaccgaaacgtaacgagacgttagagaatacgtggaagatgattatggaaggcaacaagtcaacgcttccgttgaccagttattacaagagacccgacacgttcggacttggcgaagagacaaaacaatcaggtgttttgaagaaatcggagacgtttagtgacagaactaactgttaccagtctctgccgccgccacctccgccgctagtgaaggtgaagaaggtgaaagtgtcacggagtagggatgagcttaaccggaaagtagaagcgtttataaaaaaatgcaacgacgagaggttcgcgtcgatgaaactggacaacgaagtggctcgtcatggtctttcttattaa

SEQ ID NO: 6 - DNA, 2460 bp, Arabidopsis thaliana
atggcggacaagctagctcttcctcttctccttccctgcactccttcctctaaaccttattctcacgaccaaaaccaccatatctctcggacgccttttcttactacgtctctttcgtcaccacctcctccgcctgtagagcctctcctccacgatgttttccttcaccagaaccctaattccagacaacccatcagctctcaaacatctagaaaccgtaaccggactcgaattggcaagtcacgtgaccctaacctcggtaaaccttggtcttaccatggtctttctccacaaggtcagcaagttcttcgttccctcatcgaacccaattttgattccggtcaattagattctgtactctctgagctattcgagccttttaaggataaaccagagtctacctcgtcggagttactagcttttcttaaaggattaggatttcataagaaattcgatttggctctgcgtgcttttgattggtttatgaagcaaaaggattatcaatccatgttggataactctgttgttgctataatcatcagtatgctaggtaaagaaggcagagtatcttccgctgcaaatatgttcaatggtttgcaggaagacgggttttcgcttgatgtctactcttatacttcgttgatatcagcgtttgctaatagcggaaggtatagggaagctgtaaatgtgttcaagaagatggaggaagatggttgtaaaccgactttgataacgtataatgttatcttgaatgtgtttgggaaaatgggtactccttggaataagattacgtctcttgttgagaagatgaagagtgatgggattgctccggatgcgtatacttacaacactcttataacttgttgtaaacgaggctctttgcatcaggaagctgctcaggtttttgaagaaatgaaggctgctgggtttagttatgataaggttacttataatgcgttattagatgtttatggaaagtctcatcggcctaaggaagctatgaaggttttgaatgaaatggtgctcaatggattttctccgagcattgtgacttacaactccttgatctctgcatatgcgagggatggtatgctggatgaggcaatggagcttaaaaatcagatggcggaaaagggaacgaaacctgatgtttttacttatacaacacttttgtcagggtttgagagggctgggaaggtcgaatctgctatgagtatttttgaagagatgagaaatgcagggtgcaaaccaaatatttgtacttttaatgcctttataaagatgtatggtaacaggggaaagtttactgaaatgatgaagatatttgacgagatcaatgtgtgtggtctctcccccgacattgtcacttggaatacactattagcagtctttggccaaaacgggatggattcagaagtatcgggtgtattcaaggaaatgaagagagctgggttcgtacccgaaagggaaactttcaacaccctaatcagtgcgtatagccgctgtggttcgtttgaacaagctatgactgtttacagacgaatgcttgatgctggggtcactcctgacctttccacctataacactgtgttggcagctttggcccgtggaggaatgtgggaacaatctgaaaaagttcttgcagagatggaggatggtcggtgcaaaccaaatgaattaacttactgctctctacttcatgcatatgcaaatggcaaggagattggtctgatgcattctctagcagaagaggtttattctggagttatcgagcctcgagctgtgcttttgaagacccttgtcttggtttgtagtaagtgtgatcttttgccagaggctgaacgtgcattctctgagctcaaagaaagagggttttcaccagacataaccacattaaattccatggtctccatatatggaagaaggcagatggtggcaaaggcgaacggagtcttggactacatgaaagaaaggggtttcacaccaagcatggcgacctacaatagcctcatgtatatgcatagtcggtctgcagatttcggaaaatcagaggaaatcttgagggaaatactggctaaggggatcaagccagacatcatatcgtacaacacagtcatttacgcctattgtagaaatactcggatgagagatgcatctagaatattttcagagatgaggaattcagggattgtccctgatgttatcacctacaatacgtttattggttcttatgcagctgactcaatgtttgaggaggccatcggcgtcgttaggtacatgatcaagcatggttgtagaccaaaccagaacacctacaactccattgtcgatggatactgcaagctaaacaggaaagatgaggcaaaactttttgtcgaagatctgaggaatcttgatccccatgctcccaaaggcgaggatcttaggttgctggaacggatagtgaagaagtggccatag

SEQ ID NO: 7 - DNA, 2535 bp, Arabidopsis thaliana
atgaggattatgattaagggaggtgtttggaagaacaccgaagatgagattctcaaagccgccgtgatgaagtatggtaagaaccaatgggctcggatctcgtctcttctcgttcgtaagtctgctaaacagtgtaaagctcgctggtacgagtggctcgatccatctatcaaaaagactgaatggaccagagaagaagatgagaagcttctacatcttgctaaacttctgcctactcaatggagaactattgctcctattgtgggtcgtacaccatctcaatgtcttgagaggtatgagaagctccttgatgcagcatgcactaaggatgaaaattatgatgcagcggatgatccacgaaaattacgtcctggtgagattgatccgaacccagaagcaaagcctgctcgtcctgatccggtagacatggacgaagatgagaaagaaatgctttctgaagcaagagctagattggctaacacgaggggaaagaaggctaaaagaaaagctagagaaaaacaacttgaggaagctagaaggcttgcttctctgcaaaaaagaagagaactaaaagcagctgggattgatggaaggcataggaaaagaaagagaaagggaatcgactataatgcagaaattccttttgaaaagagggcacctgcgggattttatgatactgcggatgaagatcgtcctgctgatcaagtaaaatttccaactaccattgaagaacttgaaggaaaaagaagagctgatgtagaagcacatttacgcaaacaagatgttgcaaggaataaaattgctcagagacaggatgctccagcagctatattgcaagcaaacaagctgaatgatccggaagttgttaggaagaggtcaaagctgatgttaccaccaccgcagatttcagaccacgagctagaagaaattgctaagatgggctatgccagtgaccttcttgccgagaatgaggagctaacagaaggcagtgctgctactcgtgcacttttggcaaattactcacaaacaccaaggcaaggaatgacacccatgaggacacctcaaagaactcctgctggtaaaggtgatgctattatgatggaagcagaaaacctggccagattaagagactctcagacacctttgctaggaggagaaaatcctgagttgcacccttctgacttcactggggtcactccgagaaagaaggagattcaaacgcctaatccaatgttgaccccttcaatgactcctggtggtgctggtcttactccaagaattggcttgacgccatcaagggatgggtcttctttttctatgacacccaaagggactcccttcagggatgaacttcacattaacgaagacatggacatgcacgaaagtgcaaaacttgagaggcagagacgagaggaagctagaaggagtttacgctctggtttgactgggcttcctcagccaaagaacgagtaccaaatagttgcacaacctcctcctgaggaaagtgaagagccagaagagaaaattgaggaagacatgtcagacaggatagcgagggaaaaggcggaggaagaagcaagacaacaggcattgcttaagaagagatccaaggtcttgcagagagatcttcctagacccccagctgcttcattggcagtaattaggaactcgttgctttcagctgatggagacaaaagttctgttgttcctcctactccgattgaggttgcagataaaatggtaagagaggagcttctacagttgctggagcatgataatgcaaagtatccgcttgatgacaaagctgagaagaagaaaggagccaagaaccgtaccaaccgttctgcttctcaagttcttgcaattgacgattttgatgaaaatgagctccaagaggctgacaaaatgataaaggaggaggggaagtttctgtgtgtgtcaatgggacatgagaacaagacacttgatgattttgtagaagctcacaacacatgcgtgaatgatctcatgtatttccccactcgaagcgcttacgagctctcaagtgttgctgggaacgcggacaaagttgcagcttttcaggaggagatggagaatgtgagaaaaaagatggaggaggatgagaagaaggcagaacacatgaaggccaagtacaaaacttatacaaagggtcatgagaggagggcagagaccgtgtggacccaaatagaggcgacattgaagcaggctgagattggtggaacagaagtagagtgctttaaagcattgaagaggcaagaagagatggctgcatcttttaggaaaaagaatttgcaagaggaagtgataaagcaaaaggaaacagagagtaaactgcagactcgctatgggaatatgttggcaatggttgaaaaagcagaggagataatggtcggtttccgagcacaggcattgaagaaacaagaggatgttgaagattctcacaaactgaaagaagctaagctagccactggagaggaagaggacatagccatagccatggaagcttctgcataa

SEQ ID NO: 8 - DNA, 3444 bp, Arabidopsis thaliana
atggccgccgacgaactgatgccgtctcacaggtcacacaggactcccaaatcaggtcctaccgcgaggaagaaatctgaactagataagaagaagcgtggaatctccgttgacaagcagaaaaaccttaaggcgtttggtgttaaatcggttgttcatgcgaagaaagcaaaacatcacgctgcggagaaggagcaaaagcggcttcatcttccgaaaattgatcgtaattatggcgaagctcctcctttcgtcgtcgtggttcaaggccccccaggagttggaaagtctctcgtgattaaatctcttgtgaaggaatttacaaaacagaatgtacccgaggttcgaggacctattaccattgtacaaggtaaacagagaaggtttcaatttgtggagtgcccgaatgatatcaatgcgatggtggattgtgcaaaggttgctgatctagccctacttgttgtagacgggagttatggttttgagatggaaacctttgaattcctcaatattatgcaagtgcatggatttcctagagttatgggtgttctcactcaccttgataagttcaatgatgttaagaagctgagaaaaacaaaacatcatctcaagcatcggttttggactgaaatatatcatggagctaaattgttttatttatctggtctcattcatgggaagtatacgccgcgtgaagttcacaacctcgcccgctttgtaattgttatcaagcctcagccattgacatggcgaacagcacatccttatgtgttggttgatcgccttgaagatgttacccctccggagaaagttcagatggataagaaatgcgatagaaatatcactgtgtttggttacctacgtggttgtaacttcaaaaaaaggatgaaggttcatattgctggagttggtgacttcattgtagctggggtgactgctttaactgatccttgtcctttaccttcagctggcaagaaaaaagggctgagggacagggataagcttttctatgctcctatgtccgggattggagatcttgtgtatgacaaagatgctgtttacatcaacataaatagtcaccaagttcagtactctaaaactgacgatggaaagggagaacctactaataaaggaaagggcagagatgttggtgaagatttggtaaagtcgttgcagaacacaaagtattctgttgatgagaaactagataagacattcattaacttttttggcaaaaagactagtgccagttcagaaacaaaacttaaggctgaagatgcgtatcactctttgccggaaggttctgacagtgagtctcaatctggcgatgatgaggaggatatagtaggtaatgaaagtgaaatgaagcaggaaactgagattcatggtggaaggttgaggaggaaagctatcttcaagacggacttgaatgaagatgattttgaggaagcagacgatcttgaattggattcatatgacccagatacatatgattttgaggaagcagacgatgctgaatcagacgataatgaagttgaagatggtggagatgactctgcttccgattcagccgatggtgaaccaggggattatcagatagatgataaggactctggtaacatatcacaatggaaagcacccttgaaggagatagccagaaagaagaaccccaacttgatgcaaattgtgtatggagcatcatcattagctactcccttgataaatgagaaccatgacattagtgatgatgacgaaagtgatgatgaagacttctttaagccaaaaggagaacaacacaagaatttaggtggtggattggatgtgggatatgtcaactcagaggattgttctaaatttgtgaattatggatacctaaagaattggaaagagaaagaagtatgtgagagcattcgtgatcgatttaccactggtgattggtcaaaagctgctctgagagacaaaaatttaggtactggcggtgagggagaagatgatgaactttatggtgattttgaggatctagagacgggagagaagcacaaaagccatgagaacttggaatcgggtgcaaatgaaaatgaagatgaagatgcagaagtcgttgagcgtgatgggaacaatcctcgtagtcaagccgatgaaccaggatacgctgataaattgaaggaagcgcaggaaattacaaaacagaggaatgagttagaatacaatgatcttgacgaggaaactcgaattgagttagcaggattccggactggaacatacttgaggctggagattcacaatgttccttatgagatggttgaattctttgatccttgtcatccaattctagttggaggtattggtttcggcgaggacaatgttggatatatgcaggcccggttgaagaaacataggtggcataagaaagtactaaagacaagagatcctattattgtgtctattggatggagacgctatcagactattcctgtatttgccattgaagatcgcaatggcaggcatcgaatgctcaagtatactccagaacacatgcactgccttgcttcgttctggggtcctcttgtcccacccaacactggctttgtcgctttccagaacctgtcaaacaatcaggcaggatttaggataacagcgacttctgtagttctggagtttaatcaccaggcccgtattgtaaagaaaatcaagctggttgggactccgtgcaagatcaagaaaaagactgcatttatcaaagacatgttcacttctgaccttgaaatagctcgatttgaaggttcatctgttcggacagttagtggcattagaggacaagtaaaaaaggctggaaaaaacatgcttgataacaaggctgaagaagggattgcgaggtgtacctttgaagatcaaatccatatgagcgacatggtattcttaagggcttggactacagtggaagttccacaattttacaatcctctaacgacagccttgcaaccccgcgataagacctggaatgggatgaaaacttttggcgaactccgtagagagctgaatattcctattccagtgaataaggattcactctacaaggcaatcgaaagaaagcaaaagaagttcaatccactacagattccaaagcgtctagaaaaagatttaccgtttatgtcgaaacccaaaaatataccaaagcggaaaagaccatcactagaggataaaagagcagttataatggaaccgaaagaaagaaaagagcatactatcatccagcaattccagctgcttcaacatcacacgatgaagaagaaaaaggcaacggatcagaagaagaggaaagagtatgaagcagagaaagctaagaatgaggaaataaataagaaacgtaggagagaagagagacgggacagatatcgtgaggaagataaacagaaaaagaagacgagaagaagccttgattaa

SEQ ID NO: 9 - DNA, 729 bp, Arabidopsis thaliana
atgggaagaggtagggttcagctgaagaggatagagaacaagatcaataggcaagttactttctcaaagagaaggtctggtttgctcaagaaagctcatgagatctctgttctctgcgatgctgaggttgctctcatcgtcttctcttccaaaggcaaactcttcgaatattccaccgactcttgcatggagaggatacttgaacgctatgatcgctatttatattcagacaaacaacttgttggccgagacgtttcacaaagtgaaaattgggttctagaacatgctaagctcaaggcaagagttgaggtacttgagaagaacaaaaggaattttatgggggaagatcttgattcgttgagcttgaaggagctccaaagcttggagcatcagctcgatgcagctatcaagagcattaggtcaagaaagaaccaagctatgttcgaatccatatctgcgctccagaagaaggataaagccttgcaagatcacaacaattcgcttctcaaaaagattaaggagagggagaagaaaacgggtcagcaagaaggacaattagtccaatgctccaactcttcttcagttcttctgcctcaatactgcgtaacctcctccagagatggctttgtggagagagttgggggagagaacggtggtgcatcgtcgttgacggaaccaaactctctgcttccggcttggatgttacgtcctaccactacgaacgagtag

SEQ ID NO: 10 - DNA, 1134 bp, Arabidopsis thaliana
atgaagaagaaggtgtctcagcagaagttactgtacagatggaagaggaaggtatacgccacgttgatgttcgctttctgctttgggactttcgtatttatacaagctcgtttcgcatctatacaagctcgtttcaatcgaatctctgcgtctctcgattcgcttaaaaagcctcgtctagatcagagaccacagattgccttcctcttcattgcccggaatcgactccctctcgagtttgtctgggatgctttctttaagggtgaggatggaaagttctcaatatatgttcattctagacctggatttgttctcaacgaggctacaacgcgatccaagtactttttggatcggcaacttaatgacagtatacaggtagattggggtgaatcaaccatgattgaagcagaacgtgtattgcttagacatgcacttagagattcatttaatcaccgctttgtttttctttctgatagctgcatacctctgtacagtttcagctacacgtataactacatcatgtcaacaccaactagtttcgttgatagctttgcagatacaaaagatagccgttataatcctagaatgaatcccattattcctgttcgtaactggagaaaaggatcacagtgggtcgttctgaatagaaaacacgcagaaattgtggtgaatgatacctctgtctttcctatgtttcagcagcattgcaggagaaaatcacttccagagttttggcgagatcgtcctgtaccagctgaaggttggaaggaacacaactgtatacctgatgagcactatgttcagacattgctatctcaaaagggtgtagatagcgaactcacacgaagatcactgacacactcagcttgggacctttcatcctcgaaaagtaatgaacgtcgtggatggcatcctatgacttacaagttttctgatgctactcctgatcttatacagtccattaagggaatcgacaatatcaactacgagactgaataccggcgagaatggtgtagcagtaaagggaaaccatcaccgtgcttcctcttcgccaggaagttcactcgtcccgccgctctccgcctactccgtgaaactatcttgttagagggcaaagagcatgacaataagtag

SEQ ID NO: 11 - DNA, 2106 bp, Arabidopsis thaliana
atgtcgttaaaactcaacactccttttccaattttcgcgccatctctatttcctaatcataacccaagagcacccagcgagatccgattctctagatggggcaacgctaatgccgaacggttcgagcagcgtcgccggagccaagaagaactcgaggctgagatccgtcgggaccgccgattcgacgccgctactaaaatcgtccatacccatgattccgaagcagcagctgctgagcctaaaacgtcaccgtttagatcaagaggcactccttcacttccctctgctcgttcgattccgggtcgaagatccaaatactccaaacccgattcaggacccaatagacccaagaacaaacctagagtacccgattcgccgccgcaactagacgctaagcctgaggttaagctaagcgaagatggattaacttacgtcatcaatggagctcctttcgaattcaagtacagttacacggagacgccaaaggttaagcctttgaagcttcgtgagcctgcttacgcgccttttggacctacgactatgggaaggccatggactggtcgtgctccgcttcctcagtcgcagaagacgccgagagaattcgattcttttcgattgcctcctgtggggaagaaagggctgaagccggtgcagaaaccgggtccttttcgacccggggtaggtccaaggtatgtttactccaaggaggagattttaggagagccattgacaaaggaagaggtcagagagctggttacttcttgcttgaagacaacaaggcaattgaatatgggcagagatggtttgacgcataacatgttgaacaacatacatgatctatggaagcggcgaagggtttgtaagattaaatgcaaaggagtttgtacagtcgatatggataatgtttgcgagcagttagaggagaaaattggtgggaaggtgatatatagaagaggaggtgtgcttttcctattccgtggcagaaactataaccacaggacaagaccgcggttccctcttatgttgtggaagcctgtagcacctgtttatccaaggctaattcaacaagtgcctgagggtttaactcgtcaggaagctaccaatatgcggaggaaaggacgagagctcatgcccatttgcaagctagggaagaatggtgtgtattgtgatcttgtgaaaaatgttaaagaagcatttgaagtttgtgaattggttcggatcgattgtcaagggatgaaaggcagtgattttaggaaaatcggtgccaaactcaaggatcttgttccatgtgtgctcgtatcttttgaaaacgagcagattcttatctggagaggacgagaatggaaatcgtctctcacaactccagataaaaagggtgatatccttgaagatatcgaagttgatactgccttgccagaagatgacgaaccatcggtgtcaccaaatcagagtcaaactatgacccagaaccctcctctggattctatggaactgcaaaatgatccagatggtcacgatttgagcccttcaactgtagattcctcggaaatggaaggcacaatcaattctttacagagctggtctacaaaagatgtaactgagccaacggtagatagttttcttcgagaccttgaagaacctgaagacgaaccagaaacatcggaagagatcagcaaacaaagcatagagagagttctgattttgatgaaacaagctgtggagagcgggactgcacttgtgttagatgctgctgatctggacgcagacacagtcttttcaaaagctgttgccttttcgagtgtagcttcaccaggaccagttttccagcatggcttgagaaaacaaccaacggttaagaagcaggaaagccaagaattcgggtacggagacttggaggcaaaatcaagtaatgtagtggtttctaggaatgcttccaaatcaagtaatgttgtggtttttgggaaaagagaagttgcagagaggggggaaagagaggagaaggaggagggatcgaagaagaaaatggacgagtttgctgaagattacagagaagtgatgccgcatggaacattgaaggtagatgaactagctaaactacttgcataa

SEQ ID NO: 12 - DNA, 1746 bp, Arabidopsis thaliana
atgttttcgttatcgttaatccaaccgcgtctccggatttcagagattccggtgactcaatcctacaaatctccgacgatatgttacagtagcgattcaagaactaagcgagaggaacagagacacgtgagattacctgggtttcgattagtttctggaaagagagcatctttcgattcgggttttagtggtttcaaaggagagaatgtgaatcaggatgattcgtcttctttcgatagcgaaagagttgattatgctctgttagcggagtggctacagtcttctaatgggatgcgactcattaaaaggatccatgcgatggcgttgaaacttggtgatttggtttatgcacgtaaagtgttcgacagtatgcctgagaaaaatactgttacttggactgctatgattgatgggtatttgaagtatggtcttgaggatgaggcttttgcactgtttgaggattatgtgaagcatggaatacgttttacgaacgagaggatgtttgtgtgtttgttgaatctgtgtagtaggagagcagagtttgagttagggagacaagttcatggtaatatggtgaaagttggagtggggaatctcattgtggagagttctcttgtttatttttatgcgcaatgcggtgaattgacaagtgcgttacgagcttttgatatgatggaggagaaagatgtgatatcttggactgctgttatatcggcgtgttcgagaaaagggcatggaattaaagctataggcatgtttatcggaatgttgaatcactggtttttgcctaacgagtttacggtgtgcagtattttgaaggcttgtagtgaggagaaagcgttaagattcggaaggcaagtacacagcttggttgttaagaggatgataaagacagatgtttttgtgggaacttcgctgatggacatgtatgctaagtgtggggagatttctgattgcagaaaagtgtttgatggaatgagtaatagaaacacggtcacatggacttcgattatagctgctcatgctcgggaaggttttggtgaggaagctatcagcctcttccggataatgaagaggcggcatttgattgctaacaatttgacagtagaacttcatgcacagattatcaagaattcgatcgaaaagaatgtctatataggaagtactttggtgtggctgtattgtaaatgcggagaatctcgtgacgctttcaatgttctccagcaattgccatctagagatgtggtttcatggaccgctatgatctctggatgttcgagcttaggacatgaatcggaagcgctagacttcttgaaagagatgattcaagaaggtgtagagccaaacccatttacatactcctcggctttaaaagcttgtgcgaattcagaatctcttcttatcggtagatcaatccattccattgcaaagaagaatcatgctctatcaaatgtctttgtgggaagtgctttgattcacatgtatgcaaaatgtggatttgtctcggaagcttttcgggtttttgacagtatgcctgagaagaacttggtttcatggaaggcgatgataatgggttatgcgaggaatgggttttgcagggaagcattgaagctaatgtatagaatggaggcagagggatttgaagttgatgattatatatttgcaacaattctctctacttgtggagatattgagctcgatgaagctgttgaatcttctgcaacttgttacttggagacatcttga

SEQ ID NO: 13 - DNA, 843 bp, Arabidopsis thaliana
atggatctagaagattgggaaatactccccaaaatcaactacaagggtctcgaacttgatctcggtcatgaagaggatcatgaagttacgaagatgatgagaaacaccgcaaaaagcttcgacagtgattacttcatctgcccaattcaagattctgtcggaaagacagagtttcttcatcagagatctagcgtggtccccacacaactcctccagattccaataacttgggaacctttgtcccccgtggacgacaaagatcacaataagtacctggatccggatttctcggaaccagacccggaacttttgacggagtcttttccgtcgccgagaataaccttcaagaaatcgaaggaaaccgaatttgccgacatgaaaatagattcaccagcagcgaggttcactagtcctctgccgcagaacgatgagagacactctgactcagaaggagggttaggaggagagtcttatgatgagatcatgggatcagaggttgaagaaagcagtgacttgagtagcaagaaagaggttgattgggatgaaggtgaaagaacgaatctgtggaagaagggtcttaatggaattggagctatatgttcatttggtgttgcagctgctgcagccaccatatgtgtcttcttccttggacacaacagtagcatccaaggtggtcggaacaagaaccagatcctcaggttccagatttactctgatgataataagcggatgaacgaggtagtgaaacatgcaacaaagctaaatgaagcaatctctgtgatgaaaggtcttccggtggcaagagctcaaatatcttttggaggatactacgatgcactttga

SEQ ID NO: 14 - DNA, 1818 bp, Arabidopsis thaliana
atggcggatcctctaaacggcaagtccttctttatctgtttctcccttttattctccttcactctgcttttcatttcgccgttgtatgccaccgagtctccggttatcgaagatgtttctaccgatgttgctgtgtctgttagcgaaaccaatcgagaagctgttctattgcataatttagaggaactcgttaagaatctgacggaattagtcgctaatctagatgctaagttatctgcaactccattaaaggagaagaacgagatctcagttgatgatgacatcggagaagagaaagagagaggaagggctaaggcgttttcagtgactaaatacagtccgttttggtcggagaggtttcagtttacatcagctgtgaaactcaattccgatgcgacttgtatcaatgtgttgccgtttagagatttcgaaggttcaagcaagtactttgcaattggtgattctaaaggtagagtttatgtgttcttgagaaatggtgatgttttgattgagtttttcaccactgttgattctccggttactgctatggtttcgtattcatctgtgtttaagaactcgagtttcgtggttacgggtcatcagaacggtgcggtgttgttgcatcggattcacgagggatcgaatggcgaagattggaactcgaattcggtttctatggaacatgttgggaagtttgatgtggatgattcagctgatcctgtgactttgttggaagtgcatcatgtgggtcgtgttaggtatatattggcgactgatttaagcgggaagctcacggttttaactgagaacaggacggtttatgggtcggttattccatcgagtagaccgctcgtgttcttgaagcagagattgttgtttcttactgagtctggtgctggttccttggacttaagaagcatgaagataagagaaactgagtgtgaaggactgaaccattcgcttgcgagaacttatgtttttgatgctgcggaacggtctaaagcttatggattcacatccgagggcgagatcattcacgtattgcttcatggagatataatgaacttcaaatgtagggttagatccaagaagaagtttcaaatggaggagccagtagctttacaatcaatcaaaggctatcttctagttatcaacgaagaaaaggttttcgctttcaatgtatcgactcagcattatgttcgtactgcgggtcctcggcttttgttctcagcgggattagaagagatcagatccgcgttcttgagccatcgcgaatcatcttcacgaaccaccacagtagtaaagactaggccgttaatagctagcgacagggaaaaccttcttgtgatcggtttagaaaacggatatttcgctgtttacaaatcgaagctgccaactctcaaaggagacttcaacacaatgctttggagcagtcctgtgttcttcttcatactatttctattcggggcttggcatttctttgccaagaagaaagaatcgctcactgcatggggaccagatgatccttttaccccgaccggcgcacaaaatagttcggcgaaagagccaacatttactgaaccttcaagaagaaacgatgacctcatggatctacggagaaggtacgctggtggctcataccggtcagttggagctaacgacccgagttcaagagctccggttgatggaaactatagaacaaccgcacaggatcataacaattatcgcggtggtggctcgggtcttgattcaaacgggtttggtaatagaagagatcatttgtttggtaacaacaaagttttggataacgaaagttag

SEQ ID NO: 15 - DNA, 255 bp, Arabidopsis thaliana
atgcaagacgccgagacatcacgacagccggcgaagtctttgtccgatcgagtgaagactaactgtttatccatggcagtaacatgccaggaagggtttagctatgtcaaagccttttttgttggccagacaaagagattgacggcaaagaacgagaaggaagctacggaggctcatctaacggagacaaaaatgcaagttgacgcaaccgatgaagcagagaatgccaagaagagacttcatcaatcttcttaa

SEQ ID NO: 16 - DNA, 1737 bp, Arabidopsis thaliana
atggctttgcgtttaggtgtttctataggggcagctttgggttcctctcattgggacgacggacaacgagtacgacaacgtgacttctccgcttctgtgaatttcaccgcaccggttacgagccggaggagcttaaggggtagtagaaccggtgtgaggattcttagggtttcaaatgaaggacgcgaatcgtacctcgatatgtggaagaacgctgttgatcgcgagaagaaagagaaggcctttgaaaaaattgcagagaatgttgtagctgttgatggtgagaaggagaaaggaggagacttggagaagaagagcgatgagtttcagaagatcctcgaggtttccgttgaggaaagagatcggattcagcgaatgcaggtcgttgatcgtgccgctgccgcaatctccgcagctagagctattctcgcctctaacaattccggcgacggcaaagaaggattcccaaatgaagacaacactgtcacaagtgaagtcacagagacaccgaaaaatgctaaacttggaatgtggagcagaacagtgtatgtgccacggtcagaaacttcagggactgagacaccaggaccagatttttggtcatggacacctcctcaaggtagtgaaattagttctgtggacttgcaggctgtggaaaagcctgctgagtttccaactttgccaaatcctgtattggagaaagataaatcagcggattctctttcgataccatatgagagtatgctttcttctgaaagacatagctttactatcccgccttttgagtctttgattgaggttcgaaaagaggctgagacgaagcctagctccgagactttatcgacagaacatgaccttgatctcatatcttcagcaaacgcggaagaagtagctcgtgttcttgatagtttggatgaatcttcaacgcatggagttagcgaagatggattgaagtggtggaagcaaacgggtgtggagaaaagacctgatggtgtggtttgcaggtggacaatgatacgtggggttactgctgatggtgttgttgagtggcaagataagtattgggaggcttctgatgattttgggttcaaggaacttggttctgagaaatcaggacgtgatgccactggaaacgtgtggcgtgagttctggagagagtcaatgagccaggagaatggtgttgtgcatatggagaaaactgcagacaaatggggaaagagtggacaaggtgatgaatggcaagagaaatggtgggagcattacgatgctaccggaaaatcagaaaaatgggctcataagtggtgcagcattgaccgcaacacgcctcttgacgctggccacgctcatgtctggcacgagaggtggggagagaagtatgacgggcaaggcggaagcacaaagtacacagacaagtgggcggaacggtgggtaggtgacggttgggacaaatggggagacaaatgggacgagaactttaacccgagcgctcaaggagtgaaacaaggtgagacttggtgggaagggaagcacggcgacagatggaaccgaagctggggagaaggtcacaacggatcaggatgggttcacaaatacggaaaaagcagcagcggtgaacactgggacacacatgtaccacaagaaacttggtatgagaagttccctcactttggcttcttccactgttttgacaactctgttcagctccgagccgttaagaagccttctgatatgtcctag

SEQ ID NO: 17 - DNA, 2301 bp, Arabidopsis thaliana
atgagtatcatgctatcaatttcccggcgccagaactcttatattctgctcaaccattctcgattcctccggcgtttttcttatgatgttgacccacggccggaaatcaaatcggagagccaggaatttgtagtagtcaaatttgtgaaaactcttcaaaataccccgcaacatgattgggcgtcgagcgagtcgctaagtgcgcttgtcgtatcttcttcttctgcttctcctttagtattctcgcaaatcacgcggcggctaggatcgtattctctagcaatctcgttcttcgagtacctggatgcgaagtctcagtctctgaaacgccgtgaagaatctctctccttggcgcttcagtcggtcattgaattcgccggtagtgaaccggacccgcgtgataaacttctccgtctctacgagatcgccaaagagaagaacattcctcttactatcgttgccactaagcttctgatccgatggtttgggcgtatgggtatggtgaatcagtcggttcttgtatacgaaagactcgattcgaatatgaaaaactcgcaggttcgtaatgttgtggtagacgtcttgctaagaaatggacttgtggatgatgccttcaaggtgctcgacgaaatgcttcagaaagaatctgtttttcctcctaatagaatcacagcggatattgtgttacacgaggtttggaaggaaaggcttttgacagaagaaaagataattgctttgatttcaagatttagctctcatggtgtctccccaaactctgtttggttgactcggtttatatcaagtctatgcaaaaatgctcgcgccaatactgcttgggatattttgagcgacctgatgaagaacaaaaccccacttgaagctcctcccttcaatgcgcttttgtcttgcttaggaaggaatatggacattagtagaatgaatgatttagtcttgaagatggatgaggtgaaaatccggcctgatgttgtgactttagggattcttattaacactttatgcaaatcaagaagggtagatgaagctctcgaagtttttgaacaaatgcgtggaaagagaactgatgatggaaatgtgattaaagctgattcgattcattttaatactctcattgacgggctctgcaaggtggggaggttgaaagaagcagaggagttattggtaaggatgaaactggaagagagatgtgtgcccaatgcagttacttacaattgcttgattgatgggtattgcagagctggaaagcttgagacggctaaagaagtcgtttctcggatgaaagaggacgagattaaacctaatgtggtaactgttaatacaatcgttggtgggatgtgcaggcaccatggattgaacatggcggttgttttctttatggatatggaaaaggaaggcgtgaaagggaatgtggttacttatatgacattgattcatgcttgttgcagcgtcagtaatgtagagaaggctatgtattggtatgaaaaaatgttggaagctggttgttctcctgatgcaaagatctattatgctttgatctctggattgtgccaagttagacgggatcatgacgccattagagtggtggagaaactgaaagaaggagggttttctcttgacttattggcttacaacatgcttattgggttgttttgtgataagaataatgcagagaaagtctatgagatgctaaccgatatggaaaaagaagggaagaaacctgattccatcacttacaacactctgatttcgtttttcggtaaacacaaggacttcgagagtgttgagagaatgatggagcagatgagagaagacgggttagacccgactgtcacgacatatggagcggtgattgacgcttattgctcagtcggcgaattagacgaagcattgaagctctttaaggacatgggtttgcactcaaaggtcaatccgaacactgtaatatacaacattctcataaacgcattttctaagctggggaatttcgggcaagcgctctctctgaaagaggaaatgaagatgaagatggtgagacctaatgttgaaacttacaatgccttgtttaagtgtcttaacgagaaaacccaaggagagacattacttaaactgatggatgagatggtcgaacagtcttgtgaaccaaatcagatcacaatggagattctaatggagcgtctctcaggttctgatgagttagttaagctgaggaagtttatgcaaggctactctgttgcttcgccgaccgagaaagcttcacctttcgatgtctttagcttgggataa

SEQ ID NO: 18 - DNA, 1116 bp, Arabidopsis thaliana
atgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgagtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatga

SEQ ID NO: 19 - DNA, 7395 bp, Arabidopsis thaliana
atggataaagagacggagattctctcccgtctcgcggcgaaccaccttcatctggctcaattcgagccattgaaggctacgttactcgctctcagggttcgtaaccctgacctcgcactcaccattctccaaaccatcgtctccaacgctggaagattcgataatgtcctctggtcacgctcttgtccttccccgtctcttctctcgttcctctccacgattgagcttctgagattcgaaaatcctacttctccttggggatttgattcagaaactctaagtttgcgtgccgatttcttgttgatggttcaggttttgatcgatagagttacagagaggattaaggaagatgaggagagtgaggatgaaaattctggattagggaattgtttaagggtgttgcaaggtgttttggagttaggtgttgagaggttgaagtttgttgttgatactagtagtagtgaaggaagtaataagattgaggaagatgcagttgtgtctttgaggagtatagtattggattactctgatgttttcgatgctttatgttgtaatattcagaggcaacttgcgggttgcgagagttacggtacatgtttggttgaggaagttcagggagaagaacagagaaaggagatgaatgaggccacatgtattggttctccggagctggataacatcaatgtgtttgctttgatacagaggaatgttcagttagcacagttggatgctatgaaaacaaagttggatgaaggtgatgagcgcggggcagctgatcgcattcgttatcttcaccttgattatggagtagagaaagagaactatcatgctgttctaaaagctctcctttcaagagttatggagaaaaaggatgaatatggtgattcctggcacatggtgcgccagaacttgctgtttatgtataaagaagctctctcatcgaattgtggagatcttgttcagatgatccagggtattcaagatgatatgctcctcccacatagccaactacatttatctctcgacaatgaacaaattccactccctcttgaatgtttccggcgatatcttgtagacttgaaaactgagagaaatatagaggacaaaagttctcctatgagcagggcaattaattcttgtctcagagatatgtatcattatgctcgtatttctggatcacatgttcttgagtgtgtgatgtgtgctgctttgtcttctgtaaagaaagagaagcttcaggaggctaatgatgttcttactttgtttccccgacttcgccctttagtagcctccatgggttgggatctattgccgggcaaaactgcaacccgtagaaaattgatgcggctactttggactagtgactcgcaagcacttcggctagaagaatcttctctttatggaaaccagacagatgaactggaacttgcatctttcgctgcttgtgtcaattctggtaaatcatggactccaaaggcatctttcttgatgcatggtaatgtgtcatccgcgcatgatgatgcggaggtggatccttttgttgaaaatcttgtattggaaaggctttcagcgcaaagtccacttcgggtattgtttgacgttgttccgggcataaaattccaagatgctatttcactgattagtatgcaacctattgcttcaactgcagaagcctggaagaggatagaagatattgaactgatgcatatgcgttatgctctggaggcaatcgttttagcactaggtgcaatggaagaggctatgaaggatgagacagatgctagtcatcgagtagtattttaccatttaaaagacctcactaaccatttggaggccattaaaaatgttccacgcaagataatgatggtgaacatagttatttcactcttacatattgatgatatccgtctcagttctacgcaaagtgcctcctcggcatgtttttctgaaaaaagtaacacacctggtttggatcctggcgatcttggtacagaaggggaaaaggaaattgttatttctttcacaaaacagctactcgatgttttacgccgcaatcttccatcacatccaattgaacaagagtgtcagctggatggtaattacagtactgatggaagacaggctttagaatggagagtatccatggctaagcgtttcattgaagattgtgaatggcgattatctgttatgcagcatcttctgccactttctgaacgccagtggggtttaaaggaggttttgagtattctaagggcagcccctgaaaaactgcttaatctctgtatgcaaagagctaagtatgacattggagaagaggcagttaatcggtttgcgttatcagcagaggacaaagctactcttgaattagctgaatgggttgataatgcgttcaaaggaacactggtagaagatgtaatgtctcgtactgctgaaggagcagctgccgtgcaagatttagattttcattctttaggttctcaattgagtccattggctatggttttactttttgcgcagtctcaagttatgttatcggaaatttaccctggaggagctccgaaggtggggtttacttactgggatcaggtccacgaagttgcaataatttctgtattgcgaaggatcttaaagcgtctgcaggaattccttgaacaggatgaccctcaaattcttcaagccagttttagtggagataccataatttcatcttgcacggaatctcatagacagggacaaaaagatcgtgctcttgcaatgctacatcaaatgattgaggatgctcataggggcaagcgtcagttcctgagtggtaagcttcataacttagcgagagcactcgctgatgaaaaaccagaagttgacgtactcaaaggggacggatcagacatggccgttgagaaggatggagttcttggtcttgggctaaaatatacaaagcaaagtcctggttcagcaaatagagccgtggatggaaatcctgtttcacatgaaacagaagacaagggaaagaagtcatttggcccattaagcaacaaaacctctacttatctatctcagtttatcctctatactgctgctattggtgatatagtagatggaactgacacaacccatgatttcaactttttctctcttgtttatgaatggcctaaagacctattgacgcgtctggtttttgatcgaagtagcacagatgcagctgcaaaagttgctgaggttatgtctgctgattttgttcatgaagtgatatcagcatgtgttcccccagtttatcccccacgttctggtcatgggtgggcttgtattcccgtcattccaaccactccatgttcccactcagagggtaaagtgctctctccttcaatagaggctaaacccaactgttatgtccgttcctcagcaacacctggtgtccctctgtatcctcttcagttggatgttatcaggcatttggtaaaaatttcaccagtacgagcagttttagcttgcgtctttggtgggagcatattgtacaatggcagtgattctatcatatctagctccttgaacgatgagtttccaagttctcctgatgcagacagattgttttatgaattttctcttgatcagtctgagaggtatcccactttaaaccgatggatacagatgcagactaatctgcatcgagtttcggaatttgttgtgacaccgaagcaaaaacctgatgacacacggattaagcctgatgaaagaactgggatcaagagacttcttgaacatgatagtgactcagagtcagatacagaagaaacattttctaaaaataacattcaaccagcattgacagacggcagtgctcgtgatggtggatcctttgaaaatggagtttgtagaactgatcctaccgttttcctttcttttgattgggagaatgaagtaccgtatgagaaagctgtaaatagactaattgatgaaggaaaactaatggatgctttagcactttcggaccgcttcttgcggaatggggcttctgattggttacttcagctcctaatcaaaagtagagaagaaaatccttcaacatcaggacgatctcagggatatggaggccagagcaacagttggcagtattgcctgcggctaaaggacaaacagctggcagcaacactggctcttaagtgttgcataggagacaagctctgcagaagtacagccacatactttcggcagatgatcgccataatagctggcaagaggttatctttcttccttttgtttgagatcatgtttggctcttggtatgctcgatgtgtcactctcaaaaatctaaatgggaaacaggtagaagccgaatgtaaagaagaccctgaaggcttggctctaagattggctgggaaaggagctgtttccgctgcgctagaagtggctgaaagtgcaggattatcaatagatcttagaagagaattacaaggacggcagcttgtaaagcttctaaccactgatccactcaatggtggtggtccggcggaagcatctcggtttttatcttcacttcaagactcggctgatgctctaccagtggttatgggtgcaatgcaattattacctgaccttcggtcgaaacagctcctggtccatttctttctcaagcgaagagatagcaatctgtcggatttggaagtcgcccggcttaattcttgggctttaggtctgaaagtgttagctgcattaccacttccttggcaacagagatgctcttcgcttcatgagcatcccaatttaatatttgaagctctgctaatgagaaaacaattacaatacgcctcactgatactcaaagagttcccagcattaagagataacaatgttatcatggcctatgcagcaaaagctatttctgtgacaattatcccaccaccaagagaacctcgaataactgtgtctgcgtcaaggttaagacagaaatcaagagcagggccagcagtaaaagcatccttcactagtagcttaagcaattttcagagagaggctcgaagggcattctcatgggctccacgtaatgctgaaaaccggacgacgtcaaaggatgtttatcgcaaaagaaagaattccggactgggggcttctgagagagctgcatgggaggcgatgacaggtattcaagaggaccagggatcatcttattcggcagatgggcaagataggctaccttctgtttctattgctgaagaatggatgctaactggcgacaaaaccaaagatgaaggtgttcgtgcatctcacaagtatgaaagcacccccgatattattctctttaaggctctactatcactttgttcagatgagctagtatcagcaagaagtgccatggacctatgcatcagtcaaatgaaaaacgtattgagctccaaacagttgtcagagggtgcgtctgttgaaacaattggccgagcatatcatgcaacggaggcatttgtgcagggtttgtcgtatgcaaaatcattgctgagaaagcttctaggtaccactgaatcgaccaataacaatggtgaaagaagtagagatgttgatgatatatcttctgatgctggcagctccagtgttggcagtcagtcaacggatgaaccatcggatgttctctcacttacagaaatctggttggggcgcgcagagttgctgcagagcctcttagggtctggaatttctacttctcttgatgacattgctgatcaattgtcatccgaatgtctacgagacagattaatatctgatgaacgatatagtatggcggtgtatatgtgcaagaaatgcaagattgatgttttccccgtgtggaaagcatggggtcttgctttgttacggatggagcgctatgctcaagctcgagtcaaattcaagcaagcctttcaattaaagggggaagacattcctgatgtcattcaggagataataaatacaatagaaggaggtccgcctgtggatgtgtcaattgtacgttccatgtatgaccatttggcgaaaagcgcacctacaattttggacgattctttatcagcagactcatacctaaatgttctgcatatgccatccactttccctcgttcagagaggtcgcggagatctctggaatcggaaaagaatagttctgtacctggttcagactttgaagatgggccccgaagcaacttggatactacacgctattctgagtgcaccaactacttgcaggaacatgctcgccaaaacctgcttgggtttatgttccgccatggtcactttaaagatgcatgcatgttattctttccgcaaagtggtcttccccctcctttgcaaacttcatctgtgggcgcagtaagcacatcttcatcacctcaacgaactgatcccttggcaactgaatatgggaccattgaaagtttgtgcgagttctgtgttggttatggagctatctcatcccttgaggaagtaattacagaaagacttgaatccgcaaagaatcaagatcaagccataaatcagtacatagctggagctcttactcgtatctgtgctttctttgagatcaaccggcatttcaattacctatacaagtttctggtactcaagaaggattatgtcacctctgggtattgttgtattcagctttttatgaattctacaactcaggaggatgctgtaaggcatcttgagcatgcaaagaaatactggtctctaactatcctcggggtacaggcacactttgaagaagcattgacagcgcgtcatagaggttcagactcaaaaaaacttgttacaaagggtgttagaggaaaaagtgccgcagagaagctgagtgaagaaactcttgttaagttgtcctcacgggtgaaaatgcagattgatgtggtgaagtccttcagtgactctgaaggagcaccatggaagcattccttgtttggaaatccaaatgattcagagacatccaggagaagatgtgaaatagtggagactcttgttgagaaaaatttcgacttagcttattctgttatatatgaattcaagctctcagctgttgatatatatgctggtgttgctacgtcactagctgataggaagaaaggcagtcagttgacagaacttttcaaaaacattaagggaacaatccaggatgatgactgggatcaggtcctgggtgctgccatcaatatatatgccaacaagcacaaagagcgccctgaccgtctcatcgacatgttaacaagcagccatcgaaaggtgctggcttgtgtggtatgtggccgcctgaaaagcgcattccagattgcatctaaaagcggaagcgtggctgatgttcaatatgtagctcatcaagccttacatgccaattcgcacacagtactcgatatgtgcaagcaatggctagctaaatacatgtaa

SEQ ID NO: 20 - DNA, 1488 bp, Arabidopsis thaliana
atgtgttttaataacattgaaactggtgatgaagtggaaaccgagaggcaagtgtttggttcatctgaagaagatgaatttcgagttgaagatactgctagaaataccaacaatgtacagatttctcaacaacagcagcaaccgctagctcatgttgtgaagtgggagaggtatctcccagttagatcgcttaaggttcttctggtggagaatgatgactcaacacgccatattgttactgcccttttaaagaattgcagctatgaagttactgctgttccggatgtccttgaagcctggagaattctagaagatgagaaaagttgcattgatcttgtcttaacagaggttgacatgcctgtgcattcaggaaccggtctgctgtccaagattatgagccataagacacttaagaacatccccgtcataatgatgtcatcacatgattctatggttctggtctttaagtgtttgtcgaatggtgctgttgattttctcgtgaaacccattagaaagaacgaactaaagaatctttggcaacatgtctggagaagatgtcacagctctagcggaagcggaagtgagagtggaatacatgacaagaagtcggtgaaacctgaaagcacccaagggtcagaaaatgatgccagcatcagtgatgaacacaggaatgaaagtgggagtagtggtggtttgagtaaccaagatggtgggagtgataacgggagtggaactcagagttcttggacaaaaagagccagtgatactaagagcacctcgccttcaaatcaatttcccgatgcacccaacaagaaaggaacctatgaaaatggatgtgcacatgttaatagactgaaggaggctgaagatcagaaggaacaaataggcacgggatcacagacaggaatgtctatgagtaagaaagctgaagaaccaggagatcttgaaaagaatgcaaagtattctgttcaagctttggagagaaacaatgatgacacgctgaatcgctcttctggtaactcacaagtagaaagcaaagcaccttcatctaaccgagaagatttgcaatcactcgagcaaactctgaaaaaaacaagagaggatagagattacaaagtcggtgatcgaagtgtgttgaggcattcaaatctctctgcattctcaaaatacaataatggtgctacttctgctaagaaggctccagaagaaaatgtggaaagttgttctcctcatgacagtcctattgcaaaactgttgggttcgagttcaagcagtgacaatcctttaaagcagcagtctagtggaagtgaccgatgggcacaaagagaagctgctttgatgaagtttcgccttaaacgtaaagagcgatgttttgagaaaaaggttaggtaccatagcaggaagaaactagctgagcaacggcctcacgtcaaaggtcaattcattcgcaagagggatgatcataaatcaggaagtgaagacaattga

SEQ ID NO: 21 - DNA, 3555 bp, Arabidopsis thaliana
atgcgtagattctctaccattgttgatcttctcatcagcaaaaaaccatcttctcaagcaaattctagaattgatctcatttgtaaaaggttccatatttcaagagttctcaataacgatttcgtagaatcaacagagagaaagaatggggttggtttagtttgtccagagaagcatgaagatgaattcgccggtgaagtcgagaagatttacagaattttgcggaatcaccattctagagttccaaaattggagcttgctcttaacgaatcaggtattgatctgcgacccgggttgatcatacgagtgttgagtcgttgtggcgatgctgggaatctaggttatagattctttctgtgggcaacgaagcaacctggttattttcatagctatgaagtgtgtaaatcaatggtgatgattcttagtaaaatgcgacaatttggagctgtttggggtttaattgaagagatgaggaagacgaatccggagttgattgagccggagttgttcgttgtattgatgcggaggtttgcttctgctaacatggtgaagaaagcagttgaggtgctcgacgaaatgcctaagtatgggttagagcctgacgagtatgtttttggttgtttgttagatgctttgtgtaagaacggtagtgttaaggaggcttcaaaggtttttgaggatatgagagagaagtttcctccgaatttgcggtattttacttcgttgttgtatggttggtgtagggaagggaagttgatggaagctaaagaagttttggttcagatgaaggaagctgggcttgagcctgacattgtggttttcactaacttacttagtggatatgctcatgctgggaaaatggcggatgcgtatgatcttatgaatgatatgagaaagagagggtttgagccgaatgtgaattgttacacggttttgatccaggcgttgtgtaggacggagaagagaatggatgaggcgatgcgggtttttgttgagatggagaggtatggatgtgaggctgatattgtgacttataccgcgttgataagtgggttttgtaaatggggaatgattgataaaggttatagtgttttagatgatatgagaaagaaaggagtcatgccgtcgcaagtaacatatatgcagataatggtggctcatgagaagaaagaacaatttgaagagtgtttggagttgattgagaagatgaagcgaagaggttgtcatcctgatcttctcatttacaatgtagtgataagattggcttgtaagttaggggaagtgaaagaagctgttcggttatggaacgaaatggaagctaatgggctaagccctggagttgatacgtttgttattatgatcaacggatttacaagccaaggtttcctaatcgaagcctgtaatcacttcaaagaaatggtaagccgaggaatattctctgcgcctcaatatggaacgctgaagtcattgcttaataaccttgttagagatgataagctcgaaatggcgaaagatgtatggagttgcatatccaacaaaacctcttcctgtgagctgaatgtatcagcttggacaatatggatccatgctttgtacgcaaaaggtcacgtgaaggaagcgtgttcgtattgtcttgatatgatggagatggatttgatgccgcaacctaatacttatgcgaaacttatgaaaggattgaataaactgtataataggacgatcgctgcagagattacagagaaggtggtgaagatggcaagtgagagagagatgagttttaagatgtataagaagaaaggtgaggaggatttgattgagaaagctaaacctaaagggaataaagaaggaaagaagaaagggacagatcatcaaaggtataagggaagagtgtctcttgctcggaaccgtcttaggtcggaaacgccatcatcttttcttgctcgagaccgtcttaggtcaaaaacgccatcatcgtctccattttcttcgaagcggcatacgcctaagacaagcgaaatagaagaagagtcgactccaaaagattcagttttgttaaaccctaaagatccttcaagcgcacctaagctcttccttgtacagcctcgtttagcaccgccaaagtatctacaagcgaagctgaacgaagcgctttgtctcgcgaattcgcttgaagagcaacgatatgggtactttgaatctgatttctttgacaaggaattgccttctcatgttgttgttcaaaaccctgtccgtagatcgtctaaacctcgcgaagaagttgatgctgttttcgtaaacgccattttgaccgctatccaacaacggaatttagagcgaatatgggcaaaacctgtcttagaccgtgtgggtcttataatcgaaatatttaatgctcatgcgcatacaaaggaagcaaaactacaggctgagttagctgctttgatgtacaataagagcagactggttcgagtgcgtggtactgatggacgccatacttttgggcagtttggagaagctgaagttgtcagtgcccgagggagagcaggaagcaagggaaccggtggcggttttgtaggtggtgcaggagaaactgagcttcagcttcaacgccgaagaatatcagaccggaggattcgcttgttatcccaaattaaagaagcccagcgaacacggctattgcagcgtgctggacgtaagaaaagagtggggttagagggtgagagttcaggaaccattgctgttgttggttacacaaatgctggaaaatcgactctgataagtgcactaacaaagactgctctctactgcaatgagcgattgtttgccacattagatcctacactcaagagtgcccatcttccttctggaaactttgtgcttcttagtgacactgtcggattcatatcagatctgcctatacagctggtgaaagcttttcaatcgactctggaagaagttgttgaagctgatctacttctgcatgtagttgattcaacagctccaaatatcgaggagcatcgttcaacagtgcttcatgtcctaaatcaaattggagtacctgaagagaagcttcaaaatatgattgaagtctggaataagattgattatgaagaagacgaagtggaggaagagaaatatctagatgatggcgaaggagtaggagaagaagacgaagacgaagctgatttaaaagctgaagaaactgttgatgcatctgaagcaacagtagatgaagaccaaatccaaaacggagacggtgacgacgctgatgggtggctattgtctgaagatgaaaatgctgacgaccctgagttctggaaagttccggaagttgctaaagtagatgctgcaaataagaaaggaccagatgttagagtttctgcattaaccggagttggtttgaaggagttgctgtatcttattgatgacaaaatgaaagagaagaagctcaagtctccgactatagtcgaaaggagtgagcttcataagcgtaaatggaggccacctcgtaacgatgatgaagaggagagattaatcccgttagatcaacgttga

SEQ ID NO: 22 - DNA, 2337 bp, Arabidopsis thaliana
atgaacattctccgacctccgacgtcatcatcatcttcgtcgtttcctccatacccaaagcccgtttcattaacccctccggtatctttcactctcatccacaaccccataaacctctgctctataaacccaccattcaccaacgctggtcgaccaattttccaacggtccgcctccggcactgctaatagctccgccgaagacctctcgtctttcttgggctctccctcagaggcgtattcaacacacaacgaccaagagcttttgtttctcctccgcaatagaaaaaccgatgaagcttgggctaagtatgttcaatccactcatctccctggaccaacttgtcttagccgtttagtttctcaattatcttatcaatccaaacccgagagtctcacgcgcgcacaatctatcctcacgcgcctccgcaatgaacgccagctgcatcgccttgacgctaattccctcggtctcctcgccatggctgcagcgaagtctggccaaacactttacgccgtctccgtcatcaagtccatgattcgttctgggtatttacctcatgttaaagcgtggacagctgcagtagctagtctctctgcttccggagatgatggtccggaagaatctatcaaactcttcatcgctattacgcgacgagtcaaacgatttggtgaccagtctttggttggtcaatctaggcctgatacggcggcatttaatgcggtgcttaacgcttgtgctaaccttggtgatactgacaagtattggaagttgttcgaggaaatgtctgagtgggattgtgagcctgatgtcttgacttacaatgttatgattaagctttgtgcgagggttggtcggaaggaattgattgtgtttgtgttggaaaggattattgacaaggggattaaggtttgtatgactacaatgcattctcttgttgcagcttatgttgggtttggagatttgagaactgctgagaggattgttcaagcgatgagggagaaaaggagagatctttgtaaggttctacgagaatgcaacgctgaggatttgaaggagaaagaagaggaagaagcagaagatgatgaagatgcgtttgaggatgatgaagactcgggttattcggctcgggatgaggtaagtgaagagggggttgtagatgtgttcaagaaattgctacctaactcggttgatccgagtggtgagccaccattgttgcctaaagtctttgcaccagactcaaggatctacacgacgttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaataggattacttataatgttctgctcaaaggatattgtaagcagttgcagatagatagggcagaggatttactaagagagatgactgaagatgcggggatcgagccagacgtggtttcctataacattataatagatggatgcattcttatagatgatagcgcaggagctctagcgtttttcaatgaaatgagaacgagagggattgcaccaactaagattagttacacaactttgatgaaggcttttgcaatgtcggggcaacccaagttggcgaatagggtgtttgatgagatgatgaatgatccaagggtcaaagttgatttgatcgcgtggaacatgttggttgaagggtactgcaggctaggtttgattgaggatgctcagagagtagtgtcaagaatgaaagaaaacgggttttacccaaatgtggcaacctatgggagtctagccaatggggtttcgcaggcgaggaaacctggtgatgctctcttgctttggaaggagataaaggaaaggtgtgcggtgaaaaagaaagaagcaccttcagattcttcttcagatcctgctcctccgatgctgaaaccagatgaagggttgttagatacactagcggatatatgtgtcagggctgcttttttcaagaaggcattggagataatcgcatgtatggaggagaatgggatacctccgaataagactaagtacaagaagatctatgtggagatgcactcgaggatgttcactagcaaacatgcttcacaagccagaatagataggcgggtagaacgaaagagagcggctgaagctttcaagttttggctcggtttgcctaattcttattatggaagtgaatggaagttaggtccaagagaagactag

SEQ ID NO: 23 - DNA, 1365 bp, Arabidopsis thaliana
atgaacccaacccaaaaacccgaaccggtttacgatatggtcatactcggagcatccggatttaccggtaagtacgtcgtcagagaagctctcaagttccttcaaacaccgtcttcttctccgttaaagtctctagctttagcgggtcgtaacccgacccgtttaacccaatctctcgaatgggccgcccgcccgaacccaccaccttcctctgtcgctatcctcactgctgatacatctgaccctgattcacttcgtcgtctctgtactcaaaccaaactcatcctcaattgtgttggaccgtttcgtatccatggtgatcctgtcgtctctgcttgtgctgattcagggtgtgattatttggatataagtggtgaacctgagtttatggagagaatggaagctaactaccatgatagagcagaagagactggctctttaatcgtttctgcttgtggttttgattcaattcctgctgaattgggtcttctctttaatgctaaacaatgggtatctccatcggttcctaaccagattgaagcgtaccttagcttggagtctgacaaaaaaattgctgggaactttgggacttatgagtctgcggttttaggtgttgctaatgcagaaaagcttaaagaattaagacgttcaagaccaagaaggccaagaccaacgatttgtggtcctcctgctaaaggaccaacattagaaaaccagaagacgattggtctttgggctttaaagctaccttcagctgatgcagtagttgttcgtagaactctcacaactctaacagagaaaccacatgggcttcctgggattaatgaaagtcctgagcagatacaaaagagagaagcattctggtcatcgatcaagcctgctcattttggtgtaaagataacgtccaaatctctctttgggatattccgatatgttacacttggagtgtcacttggtttactttccaagttctccttcggaagatggcttcttttgaaattcccttcagttttcagccttggttggttccagaagaaaggtccaagtgaagaagaggtagaaagcgctacgtttaagatgtggttcataggtcgtgggtacagcgaagagagtctagcttcacaaggagaaacaaagcctgacttggaaatcattacaagaatttcaggacctgagattggatatataaccaccccgataacacttgttcaatgcggtttgatagtcttgggccagcgcgaaagcctagttaaaggaggagtctacacacccggcattgtgtttggttcaaccgatatccagcagcgacttgaggataatggtatatcttttgagctgatttcaaagatcaagactcaaggataa

SEQ ID NO: 24 - DNA, 1398 bp, Arabidopsis thaliana
atgacaccggctattttttctccgacgactcttcctccatcaactgctacatggccatgttcaacatctcagaagctcatcaccgttagatcaccactcaagttcaagtgtagagcaacttcatcatcatcgtctatcactgactttgatctttatgacctcttgggtattgatcgaagttctgataagtctcagatcaaatcagcctatcgtgcgttgcagaaacgatgtcatccagatatcgcaggagatcccggtcatgatatggccatcattcttaacgaggcttaccagcttctctctgatccgatctcgcgccaagcctatgacaaggagcaagcaaaactagaagaactcagaggctatacagggaaaccgatatactcggtttggtgtggaccagaaacagagcaacgagctgcgtttgtggacgaggttaagtgtgttgggtgtttgaagtgtgctttgtgtgcagagaaaacatttgctattgaaactgcttacgggagagcgagggttgttgctcaatgggctgatcctgaatccaaaatcaaagaagccatcgaagcttgccctgtagactgcatttcaatggtggagagatctgaccttgctccattggagttccttatgtcaaagcaaccacgaggcaacgtgaggatcggggttggaaacacggttggtgagcgtgtctccaatgtatttgttgatgtcaagaagttccaagaaagatacgctaaagctatgagcagaaccacaaaagagacctcccagagagaagtacaaataagtgcagtagaggcgattaggtccatttccaattggctatactggagatcatcaccgtacacgaaaccattgagtccagaatcaaacatgagtctaacttttaccaaaagaaagaaagctgttgatccagatatcagaaagcttcaagatgttgtggcagcaatgaaacaagcagaccaaagcggaagaaccaaagagaaaggatcagcttacttgcttggagaagattactggagtccatcaaacgctgctcttccctcatctggaaacaacaacggttccaaagctagctcgaatccgcaagtgactcgtaagacatttccttcagaagagaaaccaactagtagaagagaaaatagaagacagttcaggataaagaaatttccaattgggacagccatagtagcagtattcttggttcagtaccaagcaagttacagagccgcctctgagctcaacgaccatatcggcggctcgctggctttatccatagttaacagtccatggcagcagatattgttagcaggagttacatggtacttcattggagcaatgttactccaacttgtggaagctgttcaacacaagctagaagataaagaaacataa

SEQ ID NO: 25 - DNA, 3522 bp, Arabidopsis thaliana
atggctagttcatcttcatctgagagatggatcgatggtcttcagttctcttccttgttatggcctccgccacgagatcctcaacaacataaggatcaagtcgttgcttatgttgaatattttggtcaatttacatcagagcaattcccagatgacattgctgagttggtccggcatcagtatccatcaactgagaagcgacttttagacgatgtgctggcgatgtttgtccttcatcatccggagcatggtcatgcagtcattcttccaatcatttcatgtcttattgatggctcgttggtgtacagcaaggaagctcatccgtttgcctctttcatatctttagtttgcccaagtagtgagaatgactattcggagcaatgggctttggcatgtggagaaatccttcgcattttgactcattacaaccgtcccatttataaaactgagcagcaaaatggagatacagagagaaattgtctgagcaaagctacaactagtggttctccgacttcagagcctaaggctggatcaccaacacagcatgaaaggaaacctttaaggcctttgtctccatggatcagtgatatactacttgctgctcctcttggtataagaagtgactatttccgatggtgtagtggtgtaatgggtaaatatgctgctggagagctcaagccgccaaccattgcttctcgaggatctggtaaacatcctcaactgatgccttcaaccccaagatgggctgttgctaatggagctggtgtcatactgagtgtttgtgatgatgaagttgctcgatatgagactgctacgctgacagcggtcgctgtccctgcacttcttcttcctccgccaacgacatccttagatgagcatctagttgctggccttccagctcttgaaccatatgcacgtttgtttcatagatactatgccattgcaactccaagtgctacgcagagacttcttcttggactcttagaagcaccaccgtcgtgggctccagatgcacttgatgctgctgtacagcttgtggaactccttcgagctgctgaagattatgcatctggtgtaaggctacccaggaactggatgcatttgcacttcttgcgggctataggaattgctatgtctatgagggcaggtgttgctgctgatgctgcagccgctttgcttttccgcatactctcacagccggcactgctttttcctccgctaagtcaagttgagggagtagaaattcagcacgcgcctattggtggctacagttcaaattacagaaaacagatagaagttcctgcagcagaagcaaccattgaagccactgcccaaggaattgcctcaatgctttgtgctcatggtcctgaagttgagtggagaatttgcactatatgggaagctgcttatggtttgatccctttaaattcttcggcggttgatcttcccgaaatcatagttgctaccccactgcaacctcctatcttgtcatggaatttatacattccactcctcaaagtacttgaatatcttccacgggggagtccttcggaagcatgcttgatgaaaatatttgttgccactgtggaaacaatactcagtagaacttttccgcctgaatcttccagggaactaaccagaaaagctagatcgagttttaccacaagatcagcgaccaaaaatcttgctatgtctgagcttcgtgctatggtccatgctctctttttagaatcatgcgctggtgtggaattagcttcacgcctactttttgttgtgttgactgtatgtgttagccatgaagcacagtctagtggtagcaagagaccgagaagtgaatatgctagtactactgaaaatattgaggcgaatcaacctgtatctaacaatcaaactgctaaccgtaaaagtaggaatgtcaagggacagggacctgtggcagcatttgattcatacgttcttgctgctgtttgtgctcttgcctgtgaggttcagctgtatcctatgatctctggtggggggaacttttccaattctgccgtggctggaactattacaaagcctgtaaagataaatgggtcatctaaagagtatggagctgggattgactcggcaattagtcatacgcgccgaattttggcaatcctagaggcactcttttcattaaaaccatcttctgtggggactccatggagttacagttctagtgagatagttgctgcggccatggttgcagctcatatttccgaactgttcagacgttcaaaggccttgacgcatgcattgtctgggttgatgagatgtaagtgggataaggaaattcataaaagagcatcatcattatataacctcatagatgttcacagcaaagttgttgcctccattgttgacaaagctgaacccttggaagcctaccttaagaatacaccggttcagaaggattctgtgacctgtttaaactggaaacaagagaacacatgtgcaagcaccacatgctttgatacagcggtgacatccgcctcaaggactgaaatgaatccaagaggaaaccataagtatgctagacattcagatgaaggctcagggagaccctcagagaagggtatcaaagatttcctcttggatgcttctgatcttgcgaatttcctcacagctgatagactcgcagggttctattgtggtacacaaaagcttttgaggtcagtgcttgcagagaaaccggagctgtctttctccgttgtttcactgttatggcacaaactgattgctgctcctgaaatccagcccaccgcagaaagcacctctgcgcaacaaggatggagacaggttgttgatgcgctatgcaatgtcgtatctgcaacgccagcgaaagcagcagcagcagttgtccttcaggctgaaagggagttgcagccttggatcgccaaagatgatgaagaaggccaaaaaatgtggaaaatcaaccaacggatagtcaaagtgttggtggaactcatgcgcaatcatgacaggcctgagtcactggtgattctcgcaagtgcatcagatcttcttctgcgggcaactgatggaatgcttgttgatggagaagcttgtacattacctcaacttgagctacttgaagccacggcaagagcaatacagccggtgctagcttgggggccatctggactagcagtggtcgacggtttatccaatctattgaagtgtcgtctaccagcaacaatacggtgcctttcacacccaagtgcacacgtacgtgccttaagcacgtcagtactacgtgatatcatgaaccaaagctccatacccatcaaagtaactccaaaactgccaacaacagagaagaacggaatgaatagtccgtcctatcgattcttcaacgccgcctcaatagactggaaagccgatatccaaaactgtttaaactgggaagctcacagcttgctctccacaactatgcctactcagtttctcgacactgcggctcgggaactcggctgtactatatccttgtcccaataa

SEQ ID NO: 26 - DNA, 1989 bp, Arabidopsis thaliana
atgcagtttcttcgacttcttacacttcttgtttcttcctacttcttcttcttcatcaacttctcctcctcactgaatccagatgggttgtctctacttgctctcaaatccgcaatcttacgagacccgacacgtgtaatgacttcctggtctgagtccgacccgactccatgtcactggcctggaatcatctgcacacatggccgagtcacctcactcgttctctccggaagaagactctcaggttacataccctctaaactcggtctactcgactcactcataaaactcgaccttgctcgtaacaatttctcaaaaccagtgccgactcgtctcttcaacgccgttaatctccgttacattgatctctctcacaactcaatctccggcccaattccggcccaaatccaatccctcaagaatctcactcacattgatttctcctccaatctactcaacggttcactccctcagtcactcactcaactcggaagcttagtcggcacactcaatctctcttacaacagtttctccggcgaaattccgccgtcgtatggccgttttccagtctttgtcagcttagatctcggccacaataatctcaccggaaaaatacctcagattggctctctcttaaaccaaggaccaacagcgttcgccggaaactctgagctctgtggtttcccattacagaagctgtgtaaagatgaaggtacgaaccctaagctcgtcgctccaaaaccagaaggctcgcaaatcctcccgaagaaaccaaaccctagcttcatcgacaaggacggaagaaagaataaaccgatcaccggatccgtaacggtttctctcatctccggagtctcaatcgtaatcggagcagtttctatctccgtatggctgattcgaagaaaattaagctccactgtgagtacaccggaaaaaaacaacacggcggcgccattggatgatgcggcggatgaggaggagaaggaaggtaaattcgtggtgatggacgaaggattcgagctcgagctcgaggatttgctgagagcatcggcttacgtcgtcggaaagagcagaagtgggattgtgtacagagtagtggccggaatgggatcaggtacagtggcggctacgtttacgtcatccaccgtcgttgctgtgagaaggctaagcgacggagatgccacgtggcggcggaaggatttcgaaaatgaagtggaggctataagtagagtccaacatccaaatatcgtacggctaagagcttattactatgctgaggacgagaggctcttgatcactgattacatacgcaacggcagcttgtactctgccttacatggtggaccctcgaatactctgccttcactctcttggcctgaaagattacttattgcacaaggaacagctcgtggcttgatgtatatacatgagtacagcccaagaaagtacgttcatggcaacctgaaatcaaccaaaatcctgcttgatgatgaattactgcctcgcatctcaggcttcggtcttacacgtttggtatcaggttactccaaactcatcggttcgctatccgccacaaggcaaagcttagaccaaacctacttaacctctgctacaacggtgacaagaatcacagctcccactgttgcttaccttgcacctgaggctcgggcttcttctggttgcaaattatctcagaagtgcgatgtctattcgtttggggttgtcctaatggagttgttgactggccgtttgcccaatgcttcctctaaaaacaatggcgaagaactcgtgcgtgttgtgaggaactgggtcaaggaagagaagccgttgagtgagattttagacccggagattctgaacaaaggtcacgcagataagcaagttattgcagccattcatgtcgccttgaactgcacggaaatggatccagaggttcgtccgaggatgagatcagtgtctgagagtctcggccggatcaaatcggactga

SEQ ID NO: 27 - DNA, 942 bp, Arabidopsis thaliana
atggcggcgacgtcgctggttctgacgtgcgcatcccctctattcagcagccctcgggttatttctgctacgaagaagctgactacagagttgtcgatttctacagctaaattccgaagaagatgctcgggaaacaatgatgaagtgcttctagaaggaatgccaccggagtattacgatgatgaatggcaagctcgacagagagagaagaccaaagaactgcggcggatgcagcgggaggaagaagaagaagaggagagaaagattgaagaataccgtgaaattggcacgaggttgaaggaatttcccgagcaagacttaaggaaagccagaaagctcgtctccagcttcatcagagctgccgaggaagtcgaagagagaattgaagaagcagccgagaaaggagaacttgacgagcttgtcctcatgatcatatggaaccggcttgaccttgctaggcgcgatgatgagaaggacgccatcagaagtcttgatcttttgtatagaagagtcgagacagagatcttaaaacggcaagcaagtcctgcaatgaaactgctgaatgatcttctaaatatgcatgatggctttgaagacgatgcttggctcaaggactgcagaaaacgaatggctgagaccttcccccgagaagaccccttcagcattctaatgccaccgggattcgacattgatatgcatcaaggacagttgcgaccgcccattgagactgagacagacaacacccttctgagagtagactttgtaagagaagtggatgcactgctacaggaagtgaggatagaggaagacgctacaactggtagcaaaggagaagggcttgatcctgaagctatagcacttaagtttaagcaacaggagaagcaacgaaccatccgccaaattgaagccattcttgatttagccctcaacttgaagtggtag

SEQ ID NO: 28 - DNA, 1278 bp, Arabidopsis thaliana
atggagaaaatgaatgtccgttttatgattgtgttgatggtaatgtctctggttctgggtttttcgtcggcagttgatttcagatggaggaaaactgcaggattctcagatagattcaccagagctgtttcttcagtcgtgttcccagttcatggcaacgtttatcctcttgggtactataatgtaaccatcaacataggacaaccaccaagaccttattatcttgatcttgatactggtagtgatctcacttggctccaatgtgatgctccttgtgttcgttgcttggaggcgcctcatccactgtatcagcctagtagtgatcttattccttgcaatgatccactgtgtaaggctttgcatttgaatagtaatcagagatgtgagactccagagcaatgtgactatgaggttgagtatgctgatggaggatcttctctcggtgttcttgttagagatgtcttctctatgaactatacacagggtctccggctcactccccgtcttgctctaggttgtggatacgatcaaatcccaggggcttcgagtcatcatcctctagatggagtattagggcttggtagggggaaagtaagcattctgtcacagcttcatagccaaggttatgtaaagaatgttatcggtcattgcctaagcagtttaggtggaggaattctcttttttggcgacgatctttatgattcttcaagagtctcatggacaccaatgtctcgtgaatactcaaaacactactctcctgcaatgggaggggaacttctattcgggggaagaacaacaggattgaagaatctattaacagtatttgacagtggaagttcttacacatacttcaattccaaggcataccaagccgtaacatatttgctaaagagagaactaagcggaaaaccgttgaaagaagcacgggatgaccacacgctgcctctatgctggcaaggacgtagaccattcatgagcattgaagaagtcaagaagtatttcaagcctctagctcttagcttcaaaacaggctggagatcaaaaactctgtttgagatacccccagaagcttatctaatcatttctatgaaggggaatgtatgtttgggaatcttgaatggcacagaaataggtctccagaacctaaacctcatcggcgatatatcgatgcaagatcagatgataatctacgataacgagaaacaatcaatcgggtggatgccagtggattgcgatgaacttgcttcactaaaagcagctcaagtatatgaatactga

SEQ ID NO: 29 - DNA, 1059 bp, Arabidopsis thaliana
atgcattgcggatgtgtattcgcttcacaaactgtgtcatcacttctcccatttgaaattaaaacctatgcgtcaaagcttcgagcttcgtcagcccaattaccgagaacccagattcagataaacccttcagatgatctctctatctacggttcagataaatctccggcgaatagagtttcgttgccgtctcatgtaaattctatcaccagtactacaaacccttttgtcaaacactgcttgaagctccgccaaagttcctcgtatcgccacgctcatggctctgttcttgtcgtcggaactatccccatcagggaagtatgtatgtttcaaacgaataagcaaggaatgaccactgaaattgagtgcctacttcttcatgaggaagctaagattccacaaggattagagagtctaagtatccgaattgttagagtaagttctttagtgatgaagaaactctctggagtgcaatctactgaatctgttgaagccattgccttgatgagaatccctagcagctttactgatcttaaagatgataaagacatcataacagactgcaacaaatggttcccttctgctcacagagttcttgttctggacagcatacaggatccagggaaccttggcacattagtcagatcagctatggcttttaattgggatggtgcatttctacttccgggttgttgcgatccgtacaacgacaaagctcttcgagcaagccgaggtgcttcgtttcagctacctatagtttccgggaattggaaccatcttaagcttctagaaaatgagttccagatgaagctattagctggtcatccagcaacgactactcagaaactgaaacctgtctccaaactttcggtagagtttgctcaatctttagcagagaagcctttatgcttgattttaggtagtgaagggaatggtttgtctgagcaggcacggaaagtatgcgtgctagtgagcattcccatggcaggtgactttgaatctcttaacgtctctgttgctggtggtattttcttgtacatgcttcaaaatcttgtttag

SEQ ID NO: 30 - DNA, 1038 bp, Arabidopsis thaliana
atggctgcgagcgatgaagttaatcttattgagagcagaacagtggttcctctcaatacatgggttttaatatccaacttcaaagtagcctacaatatccttcgtcgccctgatggaacctttaaccgacacttagctgagtatctagaccgtaaagtcactgcaaacgccaatccggttgatggggttttctcgttcgatgtcttgattgatcgcaggatcaatcttctaagcagagtctatagaccagcttatgcagatcaagagcaacctcctagtattttagatctcgagaagcctgttgatggcgacattgtccctgttatattgttcttccatggaggtagctttgctcattcttctgcaaacagtgccatctacgatactctttgtcgcaggcttgttggtttgtgcaagtgtgttgttgtctctgtgaattatcggcgtgcaccagagaatccatacccttgtgcttatgatgatggttggattgctcttaattgggttaactcgagatcttggcttaaatccaagaaagactcaaaggtccatattttcttggctggtgatagctctggaggtaacatcgcgcataatgtggctttaagagcgggtgaatcgggaatcgatgttttggggaacattctgctgaatcctatgtttggtgggaatgagagaacggagtctgagaaaagtttggatgggaaatactttgtgacggttagagaccgcgattggtactggaaagcgtttttacccgagggagaagatagagagcatccagcgtgtaatccgtttagcccgagagggaaaagcttagaaggagtgagtttccccaagagtcttgtggttgtcgcgggtttggatttgattagagattggcagttggcatacgcggaagggctcaagaaagcgggtcaagaggttaagcttatgcatttagagaaagcaactgttgggttttacctcttgcctaataacaatcatttccataatgttatggatgagatttcggcgtttgtaaacgcggaatgttaa

SEQ ID NO: 31 - DNA, 4134 bp, Arabidopsis thaliana
atgaagcgaattagggatgatatttacgcaaccgggtctcaatttaaacgtcctttgggctcttctcgtggcgaatcatatgagcaatctccaatcactggaggagggagcattggtgaagggggaatcaacactcagaaattgactaccgatgatgctttgacctacttaaaggaagtaaaggagatgtttcaagatcagcgagacaaatatgatatgttccttgaggttatgaaagactttaaggcacaaaagactgatacatctggtgtgatttcacgagtgaaggagctgtttaaggggcataacaatttgattttcgggtttaacacctttttgcctaaggggtttgaaataacgcttgatgatgtagaagctccttcaaagaaaactgttgaatttgaagaagccataagctttgttaataaaattaagacacggttccagcacaatgaacttgtctataagtcgtttctggaaatcttaaatatgtatcggaaggataataaggacatcactgaggtttacaatgaggtgtctactctttttgaggaccactcggatttgcttgaagagttcactaggtttttaccagactcgttggcgcctcatacagaagcccagttacttcgtagtcaagcccaacggtatgatgaccggggatcaggccctcctcttgtgcgtcgaatgtttatggagaaggatcgccgacgagaaagaactgttgcttctcggggtgatcgtgatcacagtgttgaccgttctgaccttaatgatgataaatcaatggttaagatgcacagagatcagaggaaacgtgttgataaggataatagagaaaggagaagccgtgatttggaagatggagaagcagagcaagataacttgcaacatttttcagagaaaaggaagtcctcgagaagaatggaggggtttgaagcttattctggtcctgcttcacattctgagaaaaacaatctaaaaagcatgtacaaccaagcatttttgttttgtgagaaagtcaaggagagattatgcagccaagatgattatcaagcattcttgaagtgtctcaatatgtttagcaatggaattatccaaaggaaagatctgcagaatttggtttccgatgtgcttggaaaattccctgatctcatggatgagttcaatcagttctttgagcgttgtgagagtattgatggtttccagcaccttgctggtgttatgagcaaaagtaggcagcagtctcctagcttcttgtctatgagtatacttttctcttttttttcgtacgttataggaatagaaataacactgcctggtacacttgctgcagaatcacttggtagtgaagaaaatttatctagatcagtgaagggggaggaaaaagatagagaacacaaacgtgacgttgaggctgctaaggaaaaagagcgatccaaggacaagtacatggggaaatctattcaagagcttgatctatctgattgtgagcgttgcactcctagctaccggcttctccctccagattatccaatcccgtctgtgcgccacagacagaaatcaggagctgctgtgttaaatgatcactgggtttctgtcacttcaggaagtgaagactactcttttaagcacatgcgcaggaaccaatatgaagaaagcttgtttagatgtgaagatgatagatttgagttggacatgctgttggaatctgtgggatctgctgccaaaagtgcagaagaattgttgaatattatcattgataagaaaataagttttgagggctccttccggattgaagaccatttcacagcactaaatctaaggtgtatagagagactttatggagaccatggtcttgacgtgacagacttaatacgtaagaatccagctgctgcacttcctgtaattctaactcgattgaagcagaaacaagatgaatggacaaaatgccgtgaaggttttaatgtggtctgggcggatgtgtatgcgaaaaaccattacaaatcacttgatcaccgcagcttctattttaagcagcaagattctaagaatttgagtgcaaaagcgctggtgtctgaagtcaaggacttgaaagagaagtctcagaaagaagacgatgttgttctgtctatttctgctggttacaggcaaccgataattcctcacctcgagtatgactatctcgacagagctattcatgaagacctgttcaaactagtccaattttcttgtgaggagatatgttctacaaaagagcagactggtaaagttctgaagctctgggctaattttttggagctgatgcttgatgttgcacccagggccaaggggtcagattctgttgaagatgttgtagaaacccagcatcagcgtgcatttaccagtggggaggctaatgagagttctgatgcgataagtttggtttctaggcaactaaaatttgctaccaatggagatgtgcatgcttcatctggggtctccaagcatggtgagactggtttgttgaatagggattcttcagggaaagaaaatttgaaggatggtgatcttgctaataaagatgttgccacctgtgctgaaaaaccccaaaaagatcaagaaattggaaatggagctgctaaaagatctggagatgttgatgaaagagtggccacttcaagttcgtctttcccaagtggggtcgaaaacaataatggtaaagtaggaagcagagattcgtcaggttcacggggcatattatccaaaccaagtgaagctatagataaagttgatagcattcaacatacgcagggagttgatataggccgaattatagtcttaggaaatggtctgcagtcagatacttctaaagccaacagtaattatgatgaatcgggtggtccatccaaaattgagaaggaggaaggtgaattatcacctgttggtgattccgaagacaactttgttgtttacgaagatcgtgagttgaaggctactgcgaaaacagaacattcagttgaagctgaaggagaaaatgatgaggacgctgacgatgaggatggtgatgatgcttccgaagctggtgaggatgcttcgggaactgaatctattggtgacgaatgttcacaggacgataatggcgttgaggaagagggtgagcatgatgagattgatggtaaagctgaaagtgaaggagaggcagagggaatggagtcacatcttatagaagacaaagggttgtttccgtcatcagaacgtgttctattatcagttaagcctctgtcaaaacatatagctgcagcggctttggttgatgagaaaaagaaggattccagagtattctatgggaatgacgacttttatgtccttttcaggcttcatcgagtgagtgcaattgattcttatgatttgctttctcacatcctgtacgagagaattctgtctgcgaaaacatattgctccggcagtgaaatgaaactgagaaacactaaagatacttgttcaccagatccttatgcaaggtttatgaatgctctgtttagtctgcttaatggctcagctgaaaattccaagtttgaggatgaatgccgagctattattggaaaccagtcatatgttttattcactttggaaaaactgatatacaaattggttaaacagcttcaagctgttgtagctgacgacatggacaataagcttcttcagttgtatgagtatgagaattcccggagacctgggaggtcttcctctccatctcgtttgtcaatccagcttatggataacataattgaaaagcccgacgcttatgcagtctccatggagcccacatttacgagttatttgcaaaatgagtttctctccaactcatcagggaagaaagagctacaggacattgtgctacaaaggaacatgcgtggatacaatggtctggatgatcttgcagtagcttgcaaggccatggaaggtgtacaagtaattaatggccttgaatgcaagatgtcttgctcttcctacaagatttcgtatgttttggacacagaggatttcttccacaggaagaagaaacagaagaagagcaacaacttgtcactggctaaattatcacagaatagaatagcaagattccacaagtttctctcagcttcaagatga

SEQ ID NO: 32 - DNA, 2976 bp, Arabidopsis thaliana
atgcttcagccaagtcctcctcactactcttcctctagagatgtcagacatcatcatcatcatcatcatcatcatcatcatctagctctgagttctaaagctagggtttttccactttcacttccctgtaacttctcctctagggtttcttttaagcttcaacttcactgcgccgcttcttcctcttcttcagtttctccacctcgatgctctaaacctaacccaagctctcgaaaacgcaaatatggcggcgtaatcccttccattttgcgttctcttgactcttccactgatattgaaacaactctagcttctctttgtctcaatttaagccctaaagaacaaactgttcttcttaaagagcagactcgttgggaaagagttcttcgtgtgtttcgatttttccagtctcaccaaagttatgttcctaatgtgattcattacaacattgtgttaagagctttagggagagcggggaaatgggatgaattgaggctttgttggattgagatggctcataatggtgttttgcctactaacaatacttatggtatgcttgttgatgtttatggtaaagctggtcttgttaaggaagctcttctttggattaagcatatgggacagagaatgcatttccctgatgaagtcactatggctactgttgttagagttttcaagaactccggtgagtttgatcgtgctgataggttctttaaaggttggtgcgctggaaaagttgatcttgatttggattctattgatgattttcctaagaatggttcagctcaatctcctgtgaacttgaagcagttcttgtcgatggagctttttaaggttggtgcaaggaatcctattgagaaaagtctgcattttgcatctggttcagactcttctccgaggaagccaaggttaacttccaccttcaacactctgattgatttgtatggaaaggcaggtcgtttaaacgatgctgctaatctcttctcggagatgttgaaatctggagtacctatagatactgtaacgtttaacacgatgatacatacttgtggaactcatgggcatttgtcagaggctgaatccttgttgaagaagatggaagagaaagggatatcccctgatactaagacatataatatccttttgtctcttcatgctgatgctggggacattgaggcagctcttgagtactataggaaaattaggaaagtaggactttttcctgatactgtaactcatcgagctgttcttcatatcttgtgtcagcggaaaatggttgcagaagttgaagctgtgatagctgagatggacagaaatagcattcgcattgatgagcactctgttcctgttattatgcagatgtatgtcaatgaaggtttagtcgtacaggcaaaagctctgtttgagaggttccagttggattgtgtgctttcgtcaacgacacttgcagcagttattgatgtctatgctgaaaagggactgtgggttgaagcggagactgtgttctatgggaaaagaaacatgtcaggccagaggaatgatgttttggagtacaatgtcatgatcaaggcttatggtaaggccaaacttcatgagaaagcactttctctcttcaaagggatgaagaaccaagggacttggcctgatgagtgcacttacaattccctattccagatgcttgctggggttgatttagtggacgaagcccagcggatcttggctgaaatgctggattcgggctgtaaacctggatgcaagacctatgctgctatgatagctagctatgtgcggcttggcctgttgtctgatgcagttgacctgtacgaggcaatggaaaaaacaggggtgaaaccgaatgaagttgtttatggttccttaattaatgggtttgctgagagtggaatggtcgaagaagcgattcaatactttagaatgatggaagaacatggcgttcagtccaatcatatcgttctgacttcccttatcaaggcttatagcaaagtggggtgtcttgaagaagctaggagagtgtatgacaaaatgaaggattcagaaggtggcccagatgttgctgcatcaaacagcatgctaagtctgtgtgcagatcttggcatagtttctgaagcagaatccattttcaatgctctcagagaaaaaggcacatgtgatgttatttcgtttgcaacaatgatgtacttgtacaagggcatgggcatgctcgacgaggctattgaagtggctgaagaaatgagagagtctggtctactaagtgactgcacttcatttaatcaggttatggcttgctacgctgctgatgggcagttaagtgaatgctgtgaactgtttcatgagatgttagttgaaagaaagctcttgctggattggggaacatttaaaacgctcttcacgctcttgaagaaaggtggggtgccaagtgaagctgtgtcgcagctacaaaccgcatacaatgaagctaaaccactggcaacaccagcaatcactgcaactctgttctcagccatgggtttgtatgcatatgcgctggaatcatgccaagagctcacaagtggtgaaattcctcgcgagcattttgcatacaacgcagtgatatacacttatagtgcatcaggagacattgacatggccctaaaggcatacatgagaatgcaggaaaaaggtctagaaccagatattgtcacgcaagcctaccttgttgggatatacgggaaagcgggaatggtggaaggtgtgaagagggtacatagccggctgacgtttggggagcttgaaccaagccaatcgttgtttaaagcagttagagatgcttatgtgagtgcaaacagacaggacttggctgatgtggtgaagaaagagatgagcattgcttttgaagctgaaagggagtgtagttcaagatctggagaagaagaagaagacgatgaagaggaaaattctgaagaagacgaggcattttga

SEQ ID NO: 33 - DNA, 2217 bp, Arabidopsis thaliana
atgggccgatatgagctacactatggaggtgatcggcggaataatgcgccggcaatgagaagagattataacggcggattgatcgccttttcgagatatttcagcttcttttctagcagaacatgttcaccggaatcatctatcaataaccagtttaggcttctctgcatcacttgtgataccctgacaacgacgcacaatttctctcagcttctcagacaatgcatcgacgaaagatcgatatcaggaatcaagactatccaagcccatatgctgaaatctggttttccggccgaaatttccggcagcaaactcgtcgacgcgagtttaaagtgtggcgatatcgattacgcacgacaggtgttcgatggaatgtctgagagacatattgtaacatggaactctttaattgcttatttaattaagcacagaagaagcaaggaagctgttgagatgtatagattgatgattacgaataatgttttgccagatgagtacacgttgtctagtgttttcaaggcgttttcagatttgagtcttgagaaggaagcacagagaagccacggacttgctgtgattttgggtttggaagtctcaaacgtgttcgttggaagtgctcttgtggatatgtatgtaaagtttggtaaaacgagggaggcgaagttagtattggaccgcgtggaggagaaagatgtagttttgatcacagctttgatcgttggttactcgcagaagggtgaagatactgaagctgtgaaggcatttcaaagtatgttggtggagaaagttcagcctaatgagtatacttacgctagtgtattgatttcttgtggaaacttaaaggatataggtaatggcaagttgattcatggacttatggtcaagtccggttttgagtctgcgcttgcttcacaaacttctcttcttaccatgtatttgaggtgcagtttggtcgatgattccttgcgggttttcaagtgtattgagtacccaaatcaggtgagttggacgtctcttatatcagggcttgtccaaaatggtagagaagagatggctctaatcgaatttagaaaaatgatgcgtgattcaatcaagcctaactcttttacattgtcaagtgctctcagggggtgctcgaatctcgcaatgtttgaagaaggtagacagattcatggtatagtgactaaatatggttttgatagagataagtatgccggatcagggctcattgatttatatgggaaatgtggatgctcagacatggcaagattggtttttgataccttgagtgaagttgatgttatatctttgaacacaatgatatacagttatgcacagaacggttttggacgcgaagcacttgacttgtttgagagaatgataaatcttggactgcagccgaacgatgtaacagtcttgagcgtactcttggcttgtaataactctagattagttgaggaaggttgcgaactctttgactcctttagaaaggataagatcatgttaacaaatgatcattacgcgtgtatggtagatttgcttggacgggcagggagattagaggaagcggaaatgcttacaaccgaggtaataaacccggatttggttctgtggaggacgctgcttagtgcttgtaaggttcatagaaaggtagaaatggcagagcggataacgagaaaaatcctagagatagaacctggggatgaaggaactctcattctaatgtcaaatctctacgcatccactgggaaatggaacagggtgattgagatgaagagcaaaatgaaggatatgaaactaaagaagaatccagcaatgagttgggttgaaatcaataaagagacgcatacattcatggctggagatttgttttcgcatcccaactctgagcagattcttgaaaatctcgaagagctgattaagaagtctaaagatttgggatatgtagaagacaaaagctgtgtgtttcaagacatggaggagactgcaaaagagagatctctgcatcaacatagcgaaaaactcgccatagctttcgcagtgtggagaaatgttggtggaagtataaggattctaaagaaccttagagtttgtgttgattgtcacagttggatcaagatcgtgtcaagagttatgaagagagaaattatatgtagagattcaaaaaggtttcatcatttcagagatgggtcttgttcgtgtggggattattggtaa

SEQ ID NO: 34 - DNA, 2748 bp, Arabidopsis thaliana
atggcgtctacagcggctgaacaagacgagagaaaaattgtatcggtagcatcgaacgctagccaggacatcaaaacggctgctgctgcatcgcggatcagtagccaaaacggcgcttctccatctccgtcgctcaactccaaggacttcatcgtctcagcagcagctaacatcgcttctcagccgttacagaactacgattcgaacgtttggggagtcctcaccgcaatttcaagcaatgctcgcaaacgccggcagggcataaatatacttttgacttctgatgagcattgcttaggacggctgccatgtcacgctagttatcaggtagaatcaaatgcaattagcgggaatcactgtaaggtattccgtaagccggtaacaggcggtgatggggatgatgtaactgtctttatggtagacacaagcacaaatggtacgtttctcaattgggaaaggttaacaaagaatggccctgaagtcagggttcaacacggtgacatcatatcgcttgctgttcctccagagcatgagaaggcatttgcatttgtataccgcgaagtacttggtaataatcctgcgctgtcctgcatgaacagaaaaagaaaagcagaggatactacttgtgaaattaagaggcagaagggcataggcatcagtggtcccaatggtccaatatctttggatgattttaagagcctccagcgttcaaacacagaactgaggaagcaattagaagcccaggtgcttaccattgacactctgcgtaatgagtcccgctcaattgttgagcaccatgaaagtgattatttgagtatctctactgaaatatctttgcatttgcaggaaataaaacagataaaagaatccactgcaaaatcatttcataatgaactgattgagctacgtgatcaattagatacgaagcagaaggaactggcgcaggtcaacaaattatcagctgaacagaagaattccatagatgaacttggtgagagagtaagcgcttctttgcaaactctcagtgaagcaaatgaagtaattcaaagtcaaaaggcatctatagctgaactgaagacggggttggatgaagagagaaaccaaagaagagaggaaagagaaactgccattgctgaactcaaagctgcgatacatagatgccaaattgaagctcaggaagaattgaaaagattttctgatgctgctatgagacacgagagggaacaacaagaagtaatcaacaaaatgaaggagtcagagaaagaaaagtcaatgcaagtcgaaacattgatgtcaaaattggaagatacaaggcagaggttggtgtgttctgagaatagaaaccgtctgctagaagctcaagtttctgaggagcagcttgcttttgctgatgcacaaaaaaaactggaagaacttgaccttcaagtaaaaagactgcaaaaggatctggacagtgaaaaggcagctcgagaagaagcatgggcgaaagtgtctgccttagaactagagataagtgctgctgttcgagaccttgacgtcgaaagacagagacaccgtggtgcaagggaaagaatcatgctccgtgaaactcagatgcgggcattttattctacgactgaggagatctcggctttgtttgcaaagcagcaggaacagctcaagactatgcagagaactctagaagatgaggataattgtgacaatacttcactagatattgatcttaatccaataaacagaagtcccaacagagctaatacgcagggagataaaagagcaacttcccatttgaattttgctgccagggcaagctcgtccacttcagggcaaaggtctaccagaaatgaagttgtggatacgtcatgtgaggatgcagatgctacccaaaagcatgattgtgaaatcatgagtcaggaaggccaaaacacccaagaagcagagtatccaagctctgataaagttgcaaagggtggctttggctcagatatagaaggtattggtacagcacccacttcgggaacagaccctgtaggaacagagcaagtcaatgaaactcaaagtccaggaaatgattatgagagaaatgatcatctgaggaagtctattattttagctggtgatacaatgcaaatagattgtgaaactcaggtacatgaaagtgttcagattgaaggagctgttctcttgttaaggaacccgaacgatcgaagggatactcaagacatagagggagtaggtactatagggacgtcggatcttctagcttctgaagttgcggggagttgggctaatagcacgaatccttctgtacatggagaaaacgaaactgaaagaagtagagaagatgaagagagtcagactcaaaaaatcaaggaagtgaccatagtacaggattctgctggtcagataggggaaagtcaaactaaaccgacaagtccaggggtcctggtcactaacaaggatgatgcagagcgtggagttattaacgagccagtggggatcactgatcaagggaagataaaacatggtactcgttcggactcagagacagagagttgttctgactctgatgatgatcatgagaaggaaaaacacaatcctgtctcagattctgatacagagggttctgatatgaatgatgacaagggatcactctcgtcggatcctgatacagaaagaagccatgaagttgatggggatcagaagaaacaagtggacaccatggacgaagacgataaagctacttag

SEQ ID NO: 35 - DNA, 2841 bp, Arabidopsis thaliana
atggcgaataatcctccgcagtcttctggtacccagggtcagcattttgttcctgcagcttcacaaccttttcacccttatggacatgtacctccaaatgttcaaagtcagcctccacagtattctcagccgatacagcagcagcagctctttccagtgagaccaggtcagcctgtgcatattacatcatcctcacaggctgtatcagttccgtatattcaaacgaacaagattctcacttctggatctactcaaccacagccaaatgcacctccaatgacgggctttgctacatctggacctccattttcttctccatatacttttgtaccatcatcttatcctcagcaacaaccaacatccttggtccaaccaaattctcagatgcatgtagctggcgtccctccagcagcaaacacttggcctgttcctgttaatcaaagcacatcacttgtttcccctgtgcagcagactgggcaacaaacaccggtcgcagtttccacagacccaggaaacttgactccgcaatctgcatctgactggcaggagcatacatctgctgatgggagaaaatgtctgtttcatggttttgggtctatgaattcgctttatctgatatatacttatctttctaggtattattataacaagcggacaaagcaatcaaattgggaaaaacctcttgaactgatgacaccacttgagagggctgatgcatccactgtatggaaggaatttacaacacctgaaggaaagaaatattattataacaaggttacaaaggagtctaagtggacaattccggaagatttaaagttagctcgggaacaagcccaactagctagtgaaaaaacgtccctttcggaagctggatctacccctctatcccaccatgctgcatcctcgtctgatctagcagttagcactgtgacttctgttgttcccagcacatcttcagcacttactggacattcttcaagccctattcaagcgggtttggctgtacctgtcacccgtcctccctctgttgctcctgttactccaacatctggtgcaattagtgacactgaggctactacaatgtactatttttccttgggaagttttgctgagaataaggaaatgtctgtgaatggaaaagccaatttgtcacctgctggtgacaaagcaaatgtcgaggaacctatggtatatgctactaagcaggaggccaaagctgctttcaagtctcttttggaatctgtaaatgttcattccgactggacatgggaacagacattgaaagagattgttcacgataaaagatatggtgctttgaggacactcggcgagcggaaacaagcgtttaacgagtatcttggccaaaggaaaaaagtggaagctgaggaaagacgaaggaggcagaagaaagctcgggaagaatttgtcaagatgctagaggagtgtgaagaactttcatcatccctgaaatggagcaaagcaatgagtttgttcgaaaatgatcagcgttttaaagctgttgaccgtcctagggatcgtgaagatctttttgacaattacattgtggaacttgagaggaaggaaagagaaaaggcagcggaggaacatcggcagtatatggcagactatcggaagtttcttgaaacctgtgactatatcaaagctggtacacaatggcgcaaaattcaagatagactggaggatgatgacagatgctcatgtcttgaaaagatagatcgtctgattggttttgaggaatacattcttgacctagagaaggaagaagaagagctgaagagagtagagaaagaacatgtaaggcgggccgagagaaaaaaccgtgatgcatttcgtacactattggaagaacatgttgctgcaggcatccttacagccaagacgtactggttggattattgcattgagttaaaagacttgccccaataccaagctgttgcatctaatacatctggttcaactccgaaagacttgtttgaagatgtcacagaagaattagagaagcagtatcatgaggataagagctatgtgaaggatgctatgaagtcaagaaaggcaaattttaaatctgctatttcagaagatctcagtactcaacagatatcagacataaatttaaagcttatatatgatgacttggttgggagagtgaaggaaaaagaagaaaaagaggccagaaagcttcagcgtctggctgaagaatttaccaatctgttgcacactttcaaggaaatcaccgtagcttcaaattgggaagatagcaaacaactagtagaagaaagtcaagagtacagatcgattggagatgaaagtgttagccaagggttatttgaggaatacataacgagtttacaagaaaaggcaaaggagaaggagcgtaagcgtgacgaggaaaaggttagaaaagagaaggaaagggacgagaaagagaaacggaaagacaaggataaggagagaagggaaaaggaaagagaacgtgaaaaagagaagggaaaagagaggagtaaacgggaagaatcagatggtgagactgctatggatgtgagcgaaggtcataaagacgagaaaagaaagggaaaagatcgtgacagaaaacatcgaagacggcatcacaacaattctgatgaagatgttagttctgatagggatgacagagatgagtcgaagaaatcatcccgtaaacatggtaatgatcgcaaaaaatcaagaaagcacgcaaactcgcctgaatcggagagtgaaaaccggcataaaagacagaaaaaagagagtagtcgccgaagtggtaatgacgagctagaggatggagaagttggggagtga

SEQ ID NO: 36 - DNA, 459 bp, Arabidopsis thaliana
atggaggagggacgtcaaaaagacttgcaattgttggaggagattatcgacaaaggtttgaaacagaagcttgtacatgcaactgcttcacgggacaagatctttgaagaacaaaaaacactctctgacttgcggaaaaacctagaaactctggagaagaatggtgtaaatagtctcaaaacaagggtcaaccttggttcagaagtttacatgcaagctgaagtgccagatactcggcacatattcatggatgtaggcctcggcttttatgtggagttcacacggcaagaagctcttgactatatagcacaaagggaggaaagaactcaaaaacaactagaagagtatactggtgttattacgcagatcaaagggcgcatcaaactggctcattaccagattcagcaaatactcaatcttcctgaagagaatccgtcatcccggcaacgtgcgttttag

SEQ ID NO: 37 - DNA, 276 bp, Arabidopsis thaliana
atgggtgatcataatagctcgcaagcttcttacatccatttggtgcatcatttgatagaagaatgtatagtattcaacatgggcaaagaagagtgtatggatgctctgttcaagcatgctaatattaagcctatcatcacttccacagtgtggaaagagctagcgaaagagaacaaagagttcttcgaggcatacgagagaagacgagaagaaataccgaccgagaaagagacagctcgaagaatccgtgatttgctttcacgaactacaatctaa

SEQ ID NO: 38 - DNA, 1599 bp, Arabidopsis thaliana
atgttcgcatgtttgcgtattggacgctttattcgtctgggtaacgttaccgttaaatccactaatttggtactgaggtgtgtctttatccgaaattttgccacccatgccgaccacctgttcgacgaattgccgcaacgagacctctcctcacttaactctcaactctcgtctcacctccgcagtggaaacccaaatgacaccttggctctctttcttcagattcatagggctagccctgatctcagctcgcacactttcactccggttctcggggcctgttccctcttgtcgtacccagaaacaggacgccaagttcacgccttgatgatcaaacaaggcgctgaaacaggaaccatatccaaaactgcgcttattgacatgtactccaagtacggacacttggttgattccgttagggtattcgaaagcgttgaggaaaaagatctcgtctcatggaatgctctgctttcgggtttccttagaaacggtaaaggcaaagaagctcttggcgttttcgcagctatgtatagagaaagagtagaaatcagtgagttcactttgtcttctgttgttaaaacttgtgcctctctcaagattttgcagcaagggaagcaagttcatgccatggtggtggtcaccggacgcgatctcgtggttttaggaactgcaatgattagtttttactcgagtgtaggtttgatcaatgaagccatgaaggtttataacagtttgaatgttcatacggacgaggtgatgttgaattctttgatatcaggttgcattcgaaaccgaaattacaaggaagcgtttctgcttatgagtaggcagagacccaatgtgagagtgctcagtagctctcttgctggctgctctgataactctgatctgtggattggtaaacagatacactgtgtcgctttacgtaatggtttcgtttcagattctaagctatgcaatggcttaatggatatgtatggaaaatgcggtcagattgtgcaagcgcgtactattttcagagctattccatctaaaagtgtggtttcttggacgagtatgatagatgcgtatgcggttaatggggatggggttaaagctcttgaaatcttcagggaaatgtgtgaagaaggaagcggagttttaccgaattcagtgacatttcttgttgttatatcggcttgtgcacacgcaggactagttaaagaaggtaaggaatgttttggtatgatgaaggagaagtatcggttggttcctggaacagagcattacgtatgcttcatcgatatcttaagcaaggctggtgagacagaagagatatggagattagtcgagagaatgatggagaacgataatcaaagcattccttgtgctatatgggtagcggtactcagtgcttgtagtcttaatatggatcttacgcgaggcgaatatgtagcaaggaggcttatggaagagacgggtccagagaacgcgagcatttatgtgttggtttcgaatttctatgcagcgatggggaagtgggatgtcgttgaagaattgagaggaaaactgaagaataaaggtttggttaaaacagcaggacacagcttattcatatga

SEQ ID NO: 39 - DNA, 438 bp, Arabidopsis thaliana
atgttacggaacatgatgatgccttggaacagtagcgatcacaatgtagttggaatgttaacaaggcatttcgccacaaaaccaaaacccaagatgaaaccgattgagctgaacacaccaccggagcaaactcagacgataacccgagtgatctttgatattttgaaggatcatggacctctaaccattgctgaaacttgggatcgtgtcaaggaagtgggattaagagggctgacgagcaagcgtcacatgaagataatactaaggtggatgagagagagacagaagctgaagctgatatgtaaccatgttggtcctcacaagcaattcttgtacactacttggttcactaaacacaacccttcttctaaattccccaagttaccaccggaaaatctcacaggaaaatcctctggccaccctcctaaacttccctga

SEQ ID NO: 40 - DNA, 1329 bp, Arabidopsis thaliana
atggctttcgttcgatatatcccttgtcggaagattccacgaaatgttgatcaattcgagctgccatgtcttggatcgcttcgagctttcttctctactcagaagctcataggggatgaaccagttctcgttcgagatttcatacacactgcattatatgatccaatacaaggctacttctctcaacggtcaaaatctgtcggggttttggagagaagcattaagttcaaccagcttgaagggaggaaagcatacatgaaactcttggaaaaagtatacaagcagagtgacatttcttggtttactccagtggagcttttcaagccttggtatgctcatgggattgcagaagctatactgcgtaccacaaatctctcagttccattaaagatatacgaaattggtggtggatcgggcacatgtgccaagggtgtattggactatataatgttgaatgctccggagagaatctacaagaacatgagctacacttctatagaaatcagtccctcacttgctaagattcagaaggaaactgttgcacaagttggaagtcatctatcaaagttccgagttgagtgtcgtgatgcatctgacctagctggatggaagaatgtggagcaacaaccgtgctgggtgataatgcttgaggtgctagataatctcccacatgatcttgtctattccaaaagtcaactttccccatggatggaagtcttggttgaaaataaaccagagagcgaagcactctctgagctatacaagcctttagaagatccactgattaagcggtgcattgaaattgttgaacatgaagatgatccggtttcaaaaccaaaagaaatttggtctaaactatttcccaaacctagacgtagttggcttccaacaggttgtttgaaactgctagaggttttacatgcaaagctgccaaagatgtccctaattgcttcggactttagcttcttgcctgatgtgaaagttcctggtgaaagagccccattggtttcaacaaagaaagatggatgtagctcagattacagtagttatctggacgcaaagggtgatgctgatatatttttcccaaccgatttctggcttctagaacgaatggaccattattgttccggttggaggaagatggaaaaagacgggacaccatcgaaaaaaggaaggaaaaggcgaactctcactcttgatacatcagcgttcatggatgagtttggtttaccttcaaagacgagaacaaaggacggatataaccccttacttgatgacttcaagaacactaagttctatcttagtgtcccaacacacaacactaagtag

SEQ ID NO: 41 - DNA, 16011 bp, Arabidopsis thaliana
atggctattgatgggagtttcaaccttaaacttgccttggagacgttctctgtacgttgtccaaaggtcgcagcttttccatgtttcacttcgattctcagcaagggaggagaagttgtggataacgaagaggtgattcatgctttaggggatgcgtttcttcacccggagtttacagttccgttggttcattgcttccttccaataataagaaatgttgtagatagagtggtgggtcttcttcgtctagtggatgatcttaagtcaagtattgactactcagacgatgtgtcatcagttttggataatgctatgacggaaggtattagtgtgattgatttttatgtccggcgtggacaaaggttggagcttcatgagtgtgcttgcttggccttcagtcgtgcgcttcatttcaatacgtctttgttagggtctattctaaattattttgagaaagctccaccaccatacgagcgaattcttgtgaaagatatagtttctgagtcgcgcatggaggctacagatgcgtacttgctttgtcttcgagtatcatatcgttttcttgtcattagacctgaagttttctctaagttgtgggattggtcttgttacttggactccatgaaaaggctctcagaatgtcctagacaacaaaggcatttcttggaaaagtatcgagatgctgtgtggtgtgggattcaaattctttctgttgttttgagatgcagtgacagattagcaggatgttttggttttgaagaggaagaagcactttcgtgcttgctacgctgggaggaattttgtcaggatatagaaatagagaaggctggattatacattcaattgcctacatacacagcgttgaagtctttgcaacaatttaatacccttgtacctggaattaacaagcgacaatcagcagggttagaagcagatgagccacagatgaagattcggaggctggacacctgggatgtcaattctttctctgaaccatttgaaatccactctagggtgaagaaatcttttgaaatggtctcattggctgttagtcaaaagcgacctgttcttctgtatggtccctcggggtctggaaagtctgccctcattaggaagttggctgatgaaagtggtaaccatgttgtatttatccacatggatgatcaacttgatgggaaaacattggttggcacttatgtgtgtactgatcaacctggcgaattcagatggcagcctggctcacttacccaggcgattatgaatgggttctgggtggttcttgaggacatagacaaagctccatcagatgttcccctcgtcttgtcatctttgctgggagggtcttgctcattcttgaccagtcaaggagaggagatacggatagcagaaactttccaactgttttcaactatatcgacacctgaatgcagtgtgtcacacatcagagacgctggaaattcgttgagtcctctatggaggagaattgttgtatatccaccagatcgtgagagcttgcaaagtatcctgggtgctaggtatcctaacctaggtcctgttgcagagaagcttattgaaacatttgaaaccatcaactctgctcttcgtccccaattttctagttcaacaactgaaaactcagctactttcagttctccaagtagattttcactgagagatctgctcaagtggtgtgaacgagttcatggcctgccctcctatgatggccatgcagtttatcaggaggcagcagatatattctctgcgtctaatatgtcagttaaaaaccgagtggcagtaagtgagattgtggctagtatttggaatgtcgctgttccagaatctcaggataagcccccaattcaggaattttccaggattctaaaaattggtagagtttctcttccacttggtgaaactgcgtcacatgatcggtctaggtttgttgaaacacgcacatctacacggttacttgagaaaatagctcgctctgtcgagtacaatgagccagttctcttagtaggagaaacagggactgggaaaacgacactagttcaaaatcttgcacactggatcggacagaaactcactgttttgaatttgagccagcaaagtgatatagttgatctattgggtggttttaagcctattgatccaaagcttatgtgcacaatggtgtacaatgaattcaatgaattggcaagagatttgaagattaaggatgattcaaaaattatgaaatggctgcaagataattttagagccaagaagtggcatacatttttgactgggttattggacattattaaaggcattgaaggtagaattactgaacgcatggaaggtaaaattggggaagcaaggtctagatctggtagaaagaggaagaaaccagaagaagagctcaaaaactgtgcgtgtctgaggacgaaagtgaataagatacgacaacagatccattcaggtggaatggtttttacctttgttgaaggtgcgtttgtgactgccctcagggaggggcattgggttttactagatgaagtgaacttagccccaccagagatattgggcaggctgattggtgttcttgaaggagtgagaggatcactttgtttagctgagagaggggatgtaatgggcattcccagacatttgaatttccgtttgtttgcttgtatgaatccagccacagatgctggtaagcgagacttgccattctcattccgaagcagatttacagagtatgctgtggatgatgacatatgtgatgatgacctggagatattcgtgagacgatttttaggtggacgtggatctgacagtaagttagtagccaacattgtttggttttacaaagaagctaaaaggttatctgaagaaagcttgcaggatggtgctaatcagaagccacagtacagcttaaggtctctataccgtgcgctagaatatgcgataaaagcagaagctattggtggttttcagaaagcattatatgatggattttccatgtttttcctctccttattggatgcttccagtgctaagatcgtggaaccgataataaagcgtatctccggggaaaatatccgaagccaaccacttcaaagatacttgggagaattaaaaggcagttctgataaatttgttggcagttatgttaagacgaagagtgtaattgatcatcttaatcatttggcgcatgccatttttattaaaagatatcctgtgctcttacaaggaccaacatccagtggaaaaacaagccttgtcaaatatcttgcagcaataagtggaaacaaatttgtaagaatcaataatcatgagcagactgatatccaagagtatttaggttcctatatgactgattcttcagggaagcttgtatttcacgaaggagcgttggtgaaggctgtcaggggtgggcattggattgtcttagatgaacttaatttggctccatctgatgtcttagaggcactaaacaggctgcttgatgacaatagggagctttttgtgcctgagctgagtgaaacaatctcagcgcatcctaattttatgctcttcgctacacagaaccctcctactttatatggtggacgcaaaatactgtctcgagcttttcgcaatcggtttgtggagattcatgttgatgaaattccagaagatgaactgagtgaaattcttactacgaagtgtagtattgctaacagtcatgcttcaaaaatggttgaagtgatgaaagacctgcaacgcaataggcagagtagcaaagcttttgctggaaaacatggttatataactccaagagatttattccggtgggcctatcgtttcaggacttatgacggtacatctcatgaagaactcgccagagaagggtattacatccttgcagaaaggctgcgtgatgacactgagaaggtagttgttcaagaggtgctggagagacatttccgtgtcagtcttgccaaagatgatttgtacaatatgcctgttcttcttgttggtgacactggaggaggcaaaacaacaatctgccaaatactaagcgatgttaagaagaaaagattgcacatccttaactgtcatcaatacaccgaaacatctgatttccttggtggattctttcctgtgagagacagatcaaaattgatcacagaatacgagaatcaagtcaaacagttggagctctctcaggcattgacgccttttggccaagatattgttatttgtggagatattagtagagctgaagtgtcgatcaaatcagtagaggtagctttggagaagtacaaaaatggttcagttataggagtggccgccacgccacaggatgttgattttcttgagaaaataaggaacaatatggtgatgctgtatcaaaaatggcgtgcaatatttgtttggcaagatgggccccttgtggaagctatgagagctggaaatatcgttcttgtggatgagatatctttggctgatgacagtgtattagaaagaatgaatagtgtgttggagacagacaggaaattgtccttagctgagaaaggtggtcccgtcttggaggaagttgtagctcatgaagacttttttgttctagccaccatgaacccgggtggtgattatggaaagaaggaattgtcacctgcgcttcgtaatcgttttactgagatatgggtccctcctattacagatactgaggagctcagaagtattgccttttctggcctgtccagtttgaaggaatctaatgttgtagatcccatcatcaacttctgggagtggttcaacaggttgcatactgggagaacgcttactgtcagagatctcctctcctgggttgcatttgtcaacatggcaactgagagtttaggaccagcatatgctattcttcatggagcatttctcgtgttacttgacggtttaagtctcggaactggtttctctggaagggatggtcaagatctgagagaaaaatgcttcgctttcctgttacaacaacttgagctttttgctagcgatacactacctttggagctttcaagaatggagctgtatggctggggtgattccaaagcaatttgtgaaaaaagtaagagtgttcgacatgagggcatgtttggcatcgatccattttttataagcaaaggtgatgaaaatcctgagattggtggattcgagtttttagcaccaactacccacaggaatgtcttgagagtattgcgtgcaatgcagctttcaaaaccaattttattagaaggtagccctggtgttggaaaaactagtctgatattggcgttgggaaaatattctggccacaaggttgtgcgcataaatctatcggagcagactgacatgatggatttgctgggatcagatttaccagttgaaagtgatgaggacatgaagtttgcttggtctgatggaattctcttgcaggctctaaaagaaggctcgtgggttttgttagatgaactgaaccttgccccacaatctgttctagagggtttgaatgcgattttggatcatcgtgctcaagtcttcatcccagaactgggctgtacctttgaatgccctccaacatttagagtttttgcatgtcagaatccttccactcaaggtggtggcaggaaaggtcttcccaagtctttccttaaccgattcacgaaagtttatgtggacgagttagtggaagatgattacctcttcatctgtcgctcactttacccatctgttcctagtccattgctttcaaagcttattgctctcaacagacagttacacgatggtactatgttatatcgaaagtttggtcacgatggctcaccatgggaattcaatctacgggatgtgataagatcatgccagtttatgcaagaggcgatacatgacttagaagttgaaagctttctcaatgttctgtacattcaaagaatgcgtactgcaactgaccgtaaagaagttctgcgtatctataaggctatttttgataaaaccccgtcgataaatccgtatcctcgggttcagctaaatcctgcgtacttagttgttggaactgctgccattaaacgaaatttaaatcagtctaatattgccagtgagcagttgaaacttttgcctgaaatccgtcaaaatctggaagctgttgcacattgtgtgcagaataaatggttgtgcatcctagtcggaccatcgtcatctggaaagacttcggtgatcagaatattggctcagttaacaggatatcctcttaatgaattaaatctttcgtctgcgactgacagctctgatctactcggatgctttgagcagtacaatgccttccgtaatttcagattggtgatgactcgagttgagcaccttgtcgatgagtataacagtctgctattacagtcttcccaggaggcccttttcagcaataggagtggcttagtttccagatggctttcctatttaaataagattgattcctctctcgtggagaacccattattcttcttgaacgactctgaaacactgtctacattagaagaggttgtagaagacctggaacaggtcttgaaagaaggtgttttacccgttagttggtcaaaaaagtatctggaacaaatctcgaagactatattgcagttacaaactcatgagaaaaagcagtctacaaagtttgaatgggtgacaggaatgctgataaaggcaatagaaaagggagagtgggttgtcctcaaaaatgctaatctctgtaatcccacggtacttgatagaattaactcattggtggaaccgtgtggatcaatcactataaatgaatgcgggatcgttaatggtgaacctgtcactgtggttccgcacccaaactttcgtttgttcctgtctgtaaatccaaaatttggggaagtatcaagagcaatgaggaatagaggcgttgaggtatttatgatggggccacattggcagctcaatgaggatggctcaaactgtgaagagcttgtgctgagaggtgtggaaaggtttcttgctctgtcaggtattccaggttataagctggttacttccatggccaaagcacatgttcatgcatggctaaacggtcaaagctttggtgtacggatcacgtatcttgagctcgaacagtgggttcacctcttccaattgctgctcatgaatggtaatcaacttttgtggagcttacagctaagttgggagcacatctatctctcttcgcttggggtaactgatggaaaagaagttgttgattttgtgcgtgagacatatttatcagatgttgaactttctgagcttgattcatttatgggtggggatctgtacctgcctggaggatggccaaagcctttcaacttgagagacttgacatggtactcaagagaaacaacagtaagacagaattgcatgtatctggagttcctaggagctcagtatgcctcacatcagcctaaaataagcgacaatgtcaaatcaagagatagggagttggctgctggggaaccaagaattatttattctattgattcttggacgcttaaaaaagtcttgtttcctaaagccttaattgggtcaagctgtgcaccagatgcagcaaattttgaaaatgatttggcttcaaaaatgctattgtttgctgccaactggacaatagaacaggcaaccgaagaggatattcaactctatcttgcgtggtttagttggtttggttctagactgcaacaacactgtccgtttctgctttgttttctcaatacgttgaaggttgagtttgagcatccaatttggaatcatatatctagatgtcggaaaaatctgaaattcctctgcagattggatccagatgctgttccaattcctatgctgtcctccaaattgattgatgtagccgcatcaaatgaccagtccaaaccttacagtaaatccctctttgagtctctcaactctgttggcgttcttcgtcgttcgtatcagcagtggcttgtagagagcaacgacaaccacacagatgtatccacttttactcggtttttggattcgcttcgcgtattggagaagaaaattctttgcgaaattgttggagcaccatctttcagtgtgttgattcagttgtacaccgaagttattgacaaccattcattcttttggtctggtttggtctcttcttcagatgagtatctattgttttccttttggtcactgataaaatctatcaaaaagatgcacagttttttccctggagaagttcaggtggttctggaggaaagcaaaaatattaacaacatagttttgcatggtcaccctgaaaagtctatgctgtgggcttatgggggacatccttccttgccggtatctgcagagctgttccacaagcagcaagagtttctacagctgtgcagcacagtttggccattgaaatcagaatcagatgaacacggaaatgatcatcttaccaaagccattccattttctggccctgaattatgtttgcttgccttggaaggtctttgcatttcatcatacattgctgacgaagacgatgtagattatgtagctgctgttcagctggatgagatctaccagacttttttggagaggctgaaactagagaagaagagactggaggataaaatgggtttcagtgagattgacaatactgaaaatataactgcttcctgctgcgtgttctgtccagagattgtgactacagggtctggatttagcagttgggtgaagacatgttttattgctagcagtgaaagttgttctctagacgtagagttacttgctgcacttcagcacctcttggttgctcgacctactgaacatcaggatcttgtggacattcgaaaactgctcaaaccggctctagaatattctttatcctcaaccaggcctccacagactcttgtagctcatcaaaaactcctgtgggcaattgatgcacatgcctctgaactaggagtggacaccaaaattgctggttttgctctcgagatttggtactggtggcattctgtattgtggaaaaatagtcaaattggtctcatgaatatctcagacactggcaactgtcagattctgtcaccttctatgctgattcagcctgtgaaaacagctaccgttgctcagattctggaaaatgtattttctgttaaggattattctgttcaatcaatgaaacttctttctgcttcacgatatctatggaaaagctcacaaccctatcaagaaatgcctggttctctattgtcaattgcacgttcccttttccaacagataatatatacgcaccaaaagtcatttgagtcagaaacgtttgtggcaattaagtctgtatttcatgcaattgagaaaaagcagaacaagatggatggaatacagaatcttatctcactgattggctcatcaagccataataaattgaaatccgttactcactcatttgtcggaccattagcaaaacgtctttattccgatagctcatcaaatgaattctactgcaatcttggcttggcgtggctttatcttggaggactacgcttccatcttttgaatagcttagatgttatagatccagccatgaagatcacttgcaagctgttaaagctagaagagaaaatctcatcacttgagctaaacatcaaggtccggggagaatgtgggtatctgtctggattgctttactctggaaacaatgacgaaagcagtgaacatacattatctaagctcaaaactgagcataaaagattgcaaagaaaggttatttttagatctgatccaaaaaagtaccaggatttacgaagggcgctggatgaatttgctggatttctcacacgtcccataagtctggtcaacgatattgaagtgcttgattggaatcaggttgttgagcaggttttcaactggcaggagacagcaatatcttttattgatcggatgtcaagtgactattctgaatatgtcgatataactcagccaattcaagtttcagtgtacgagatgaaattgggtttatcactctttgtatctggtgctctcttgggaaaacttctcaacagatttgacatagacatggttgactcagtcatggaaacaatttatgccttaatgagatttccaagggactcgtcgatagcttcaactacctacaccgaatgtttgccacctttgcacctttcccatggtgcaaattctcgtgctaagtccttaggtttggatgttggcttgttgcacaaacttatctctgtttcaagtgcagaagattcgagaaaagcctcagagttgcaactcaaagttgctctttataaaaatctccatgctcgtgttttacaatttgtcgcaaatactgggctactggatgaagcttcttttgagttattggacaagatatatgttgaattggcgagaatttggatggagatgaagtttcaagccaaaacaaaggctgacaatcttcctgggctgtacaaatttcgttcccgggacttcaaaattgatagtgtcatggaagtagatatatctgcccttggcaagtatttcccaaacgaaagtttctctgagtggcaagagtatctggctgatgatgatacgaagaatgtgaaagatatgacacatattgaccaggatgaggaaaatttggaggatgattgggacttgatacaggagcatctggatagtatatatagcacacataatgagttatttggtttctgtgacctctctgaaaagtctggaagattctgtattactgacagtagaagactggattcgttcactgattcctatgaacttggagtcagtatgatcaaagggctaaggggtttatttacatcgagcttggatgcaaaacttgttccagaacacctacttcgtctttgcctggaaaacaaaaaaaacttcacttcaaactatcagtcagccagtaaatataacttttacaaggatttggatggtcctgagctggggaaaatggtcaagtttctcactcctcttcaacaaagaattaattctctattgcaagaacgggaggaccatcctggtcttcagaaactttctggtgtacttcagatgctcttggctattccctccagtactcctctcgcaaaggctctctcaggattgcaatttctgctctgcaaggttcacaagttacaggaagagggatgtaaattgcccatctctgatcttttggagccaattatttccctagcaagctcttggcagaaggtggaatttgagcgctggcctactttgcttgatgaggttcaggatcagtatgaactaaacgctaggaagttgtggcttcctttgttttcagttctgtttcagaaggatgctgtggaaatttcagaacatgaaaacgagtccatttcacaaagtttggtggagttcattgaaacgtcaaatgttggtgaatttaggagacgtcttcagcttctcttttgtttccttcttcaattaagtatgggtagctcgttggggatatattcaagtgtaatggagcagttagatttgaatagaaaaaatgttgaaactgagttaaaggaggttcttaaactttgtcggtgggagaggccagataattatttgtacaatgagaccactaaaaggaccaggcaaaaggtcaagaaactgatacagaagtttacggacatgctacgcctccctgtaatgcttgttaagccagacctgacgaaggaacgagctcaatttctccctctactagatccagatcttatggatggagcatccgacatgaggatcgaggtcctagttagtgctttagatgcagagcaattgagggacaggtcttcatggtatgttgtctggtggaataaattaaaggaatcggtaggacgctttcaccaagaaatgcactataaaacattgctgatgggtgcagagcatcagtattcgtcccctgtctatcagggtgattggaaaaatttgtggagtacggttgctaggattggtgaaaccatagctggctgttcagatctatggagaaacagtgatagagatgttgcaaagaagagggccctgtttgaacttctcaagttattagaaagtagtggtttgcagaaacacaagtttgaaaatatagagatgtcaaatcactttaaagggttgctttatcagccagcatacgatccaaagcatctgttactgctaacacataccaaaagtaacatacatccttccatgggtgtagaagatcaaaacaaggaaaattcactagttgagtggagagtggcaaatgagttttactttaagagcttggcttcagtgcaactcatgttaaatattgaccgaaaacactccgatgtaacagctgagcaggttaaacgggcaatctcatttctcaatcatcttgtggaaatacaacggcaacaaaggaaatctgcgtatgcctttgccgaacttttcaaccgctttcgccaatgtgttttatctctagcgagattactgggtgattcagttggtgcggatagaaaggatgattctgtgttcagtttcccccaaaatcaacatgctgtcttcaattgcttgtggctacagaagcaactctttgataacattactgcaatgcttcttgaggagtcggccttactgagaacagttggaagtacacacttggattcctgtcaagctgtgaaaacctcatcacggagtttgctcagctttattgaaatactaattcccatcgctcaaaattccaaggcttcgctggataggcttctacttgattgcaacggttttatcatcacaccaagtagcagtcttaagcagtttgtcactcagcatatggttcaggtgctacgccagaactttgatcaacttacggaccttgagaaccaaatttcaagtttctgtgaaaacaatgagaaaagctattgcagagacgttcttctcagtcaattttcccctgtgtttaaagaggggaaattgttggctgaaaatctgaactgcttacttaacgtgagagaccagtcaactggaatggaacccaaggaacgactatttcttgaagaaaatcttgcaagtatatttgcaaatgttaaggatgtgattggaaagctttgctcttataaagatggaagtctttctcaagaagaggaaatgaatattactacatgggatggtctgtttaagaaggcagaaaatgacttgaaccttgataacctgtgtaaactcctgtccgaatcatttggttccattgaacaactgttgaactcatcaggcgtcctttcagctggtgttggagaccagttgaagcaacttcaagcatttttggatcttttattgagctttggggattgttaccttaaagagtttttggcgataagcaaaacggtttcactgataacccatgtccttgcaagtgttcttgccgatctatttacaaaaggatttggcatctccaaaaatgaagaagatgatgactctaaagttgacaaatcggaagctgcagaaggtactggtatgggagatggtgtgggggcaaaagatgtaagtgaccaaatagaagatgaagaccaactgcatggcacagataagaaggaagaggaagagaaagagcaagatgatgtgctgggtaaaaacaaaggcattgagatgagtgacgaatttgatggcaaagaatacagcgttagtgaggatgaagaagaagacaaggaagacgaaggaagtgaggatgagccgttggataatggaataggagatgtgggatctgatgccgaaaaagccgatgaaaagccatggaacaaggatgaagaagatgaggaagaaaatatgaatgagaagaatgaatctggaccatctatagtcgacaaggacacaagatcaagggagctaagagccaaggatgatggtgttgaaactgctgatgagcctgaggagtccaatacttctgacaaaccggaagaaggaaacgatgagaatgtggagcaggatgattttgatgatacagataatttagaagaaaaaatccagaccaaggaagaagcacttggtggactaactcctgatgtcgataatgaacaaattgatgatgacatggagatggacaaaacagaggaggtcgaaaaggaagatgcaaatcagcaggaagaaccttgttcagaagatcaaaagcatcctgaagaaggtgaaaatgatcaagaagaaactcaagagccatctgaggaaaatatggaggctgaggctgaagataggtgtggatcaccccaaaaagaagaacctggaaatgatcttgaacaggaaccagaaacggaaccaatagaaggaaaagaagttatgtcagaagacatgatgaaaccgaacttccgtaatgataatatttctggcgtagagtctggttcacaaaatccccatgggtctaatgtgctgggtgcaggaagtacagcaccacaagaaaatttgtctgctactgatgttacggatgaactcactgattcaatggatctgccttcgagtagtaacacggaaatgaacctcatgatgaccaacatggccaacggtgagacattgacagacaacttaccaaagatggaatttcctcaaaaccagtcatctactgctcaacaaaccaaggtcaatccttataggaacgttggtgatgccttgaaggagtggaaagaaagagttagaatctcctctgaccttggagaaaagcaagaggctgaaaatgagatggaagaccctgatgctagtgaatatggatttgcttctcagtttgatgcaggaacttcccaagctctaggacctgcgttgcctgagcaagtgaacacagatatgagagaaggggaatccgaagaagaaaaacttgcaggtaatcaggatgatgtctctccaatggatattgatgacttgaacccagaaaacaaacctgctgtccaatccaaaccatcgatcagtaatagcatcgcggaacaggtccaagaaccagatacagataggacccaccaagagaactctcctattcataattttggtgatggtaacagtaggatggactctatggtctctgtcgacaatactttcttgggggaagaggcatgtaatctggaccggatgcaagtgactgataatgactcggaaagcaatcaggataatcaggaagatccagatgccagaagcaatgctgttgttctttggaggagatgtgaattgcttactgcaaaaccgtctcaggagctggctgagcaactacgtcttatcttagaacccacgcttgctagcaagctcagtggtgactacagaacgggtaaaaggatcaacatgaagaaggttattccatacatagcaagtcactatcggaaagataaaatttggttgaggaggacaaaaccaaacaagcgtgattaccaagttgttatcgctgtggatgactcgcgtagcatgtcagaaagtggatgtggtgattttgcaattagagctttggcaacggtatgccgagctatgtcacagcttgagctgggaagtttggctgtggcaagtttcgggaagcaagggagcataaagatgttacatgattttggtcagtctttcaccacagaatccggcattaagatgatctcaaatttgacatttaaacaagaaaatctcattgaagatcaaccagtcgtcaatctgctgagaaacatgaatgaaatgctagagaatttggccagcacaagacgacagtcttacgggagcaacccgcttcaacaacttgtactaatcatcggcgatgggaagttccatgagcgagagaagttgaaacgaactgttagaagctttctccagcaaaaacgtatggtggtatatctgcttctcgatgacgcagagcaatctgtttttgatttagcggactatgtatatgatggtgaaaggagaccttataagaaaatgaattacttggattccttccccttcccatactacattgtgctaagagacatcgaagccttacccagaacacttggtgatgtgttgagacagtggttcgagctgatgcaaagctcgcgggactga

SEQ ID NO: 42 - DNA, 1005 bp, Arabidopsis thaliana
atggagactaccggagaagttgttaaaacaaccaccgggagcgacggaggcgttacggtggtgagatccaacgcgccgtcagacttccacatggctccgaggtcagaaacttcaaacacacctcccaactccgtcgctcctcctcctcctccaccgccgcaaaactcctttactccgtcggcggctatggatggtttctcaagcggaccgataaagaagagacgtgggcgccctaggaagtacggacacgacggagcagcggtgacgctatctccgaatccgatatcatcagccgcaccaacgacttctcacgtcatcgatttctcgacgacatcggagaaacgtggcaaaatgaaaccagcaactccaactccaagctcattcatcaggccaaagtaccaggtcgagaatttaggtgaatggtctccttcctctgccgccgctaatttcacgccgcatattattacggtgaatgcaggcgaggacgttacgaagaggataatatcattttctcaacaagggtctctagctatttgcgttttatgcgcaaacggtgtcgtttcgagcgttacacttcgtcagcctgattcatctggtggtacattgacctatgagggtcggtttgagatattgtcactatctggaacattcatgcctagtgactcagacgggacacgaagcagaacaggcgggatgagcgtgtcgcttgctagccctgatggacgtgtagtaggtggtggtgttgctggcttgctggttgcagccactcctattcaagtggttgtaggaactttcttaggtggaacaaaccagcaagaacagacaccgaagccgcataaccacaacttcatgtcttctccattaatgccaacttcttcgaatgtagctgatcatcgaaccatccgtcccatgacatctagtctcccgatcagtacatggacaccgtcttttccttctgattcacgacacaagcattctcatgactttaatatcactttgacgtga

SEQ ID NO: 43 - DNA, 1179 bp, Arabidopsis thaliana
atgggtactcacattgatatcaacaacttaggcggcgatacttctagagggaatgagtcaaagccattggcgaggcagtcttcgttatattccttaacgtttgatgagcttcagagcacattaggtgagccggggaaagattttgggtctatgaatatggatgagttactcaagaacatatggactgctgaggatactcaagcctttatgactactacatcttcggttgcagccccgggacctagtggttttgttccgggaggaaatggtttacagaggcaaggctccttgaccttgcctagaacgcttagtcagaagactgtcgatgaagtctggaaatacctgaattcgaaagaaggtagtaatgggaatactggaacggatgcgcttgagaggcaacagactttaggggaaatgactctggaagatttcttactccgtgctggcgttgttaaagaagataatactcagcagaacgaaaacagtagtagcgggttttatgctaacaacggtgctgctggtttggagtttggatttggtcagccgaatcaaaacagcatatcgttcaacgggaacaatagttctatgatcatgaatcaagcacctggtttaggcctcaaagttggtggaaccatgcagcagcagcagcagccacatcagcagcagttgcagcagccacatcagagactgcctccaactatctttccaaaacaagcgaatgtaacatttgcggcgcctgtaaatatggtcaacaggggtttatttgagactagcgcagatggtccagccaacagtaatatgggaggagcagggggtactgttacagctacttctcctgggacgagcagtgcagaaaacaatacttggtcatcaccagttccttacgtgtttggtcggggaagaagaagcaatacgggcctggagaaggttgttgagagaaggcaaaagagaatgatcaagaatcgggaatccgctgctagatcaagggctcgaaaacaggcttataccttggaactggaagctgagattgaaagtctcaagctagtgaatcaagatttgcagaagaaacaggctgaaataatgaaaacccataatagtgagctaaaggaattttcgaagcagcctccattgctggccaaaagacaatgcttgagaagaacccttaccggtccgtggtaa

SEQ ID NO: 44 - DNA, 2505 bp, Arabidopsis thaliana
atggttgataacagtaacaataagaagaggaaagagttcatcagtgaagcagacatcgccactcttttgcagagatatgatactgtgacgatactgaagttgctacaagaaatggcgtattatgctgaagcaaagatgaattggaatgagttagtgaagaagacaagtactggaattactagtgctagagaatatcagttgctttggcggcatcttgcttatagagattctctcgtccctgtgggaaataatgctcgagttctggatgatgatagtgatatggagtgtgaattggaagcatcccctggagttagtgttgatgtagtaacggaagctgttgcgcatgtgaaagtgatggctgcttcctatgtgccaagtgagtccgatattcccgaagactcaacggttgaggctcccttgaccattaacataccttacagcctgcatagggggcctcaggaaccatcagactcatattggtcatcaagagggatgaatatcacctttcctgtttttcttccgaaagcagctgaaggacataatgggaatgggttagccagtagcttggctcctcggaagagaagaaaaaaatggtcagctgaggaggatgaggagctgattgctgctgttaagcgacatggtgaaggcagctgggcccttatctctaaggaagaatttgaaggagagcgaacagcctcacaactctcacagcggtggggggctataaggagaaggactgatacttcaaacacttctacccaaactggcctacagcgaacagaagcacaaatggcagctaatcgtgcattatctttagcggtgggaaatcggttaccctcaaaaaaacttgcagtaggtatgactccaatgctgtcatccggtaccatcaagggagcacaagccaatggtgccagcagtggtagtacattgcaaggtcaacaacagcctcagccacaaattcaagcattatcacgggcaacaacatcagtgccagttgcaaaatctcgagttcctgtaaagaaaacaacagggaactccacttcgagagcagacctaatggtaactgctaattcagtagctgctgcagcctgtatgtctggcctggcaaccgctgtaacagtgcctaagattgaaccaggaaagaatgctgtttctgcgttggtgccgaagactgaacccgtaaaaaccgcttccacagtttctatgcctcgtccttcaggtatatcatcagcactgaatactgagcctgtaaaaaccgctgtggcagcctctttgcctcgttcatcaggtattatttcagcaccaaaggttgagcctgtaaaaaccgctgcttcagcagcctctttgcctcgtccatcaggaatgatatcagcaccaaaggttgagcctgtgaaaaccaccgcctctgtagcctctttgcctcgtccatcaggtattatttcagctccaaaggctgagcctgtaaaaaccgctgcttctgcagcctcttcgcctcgtccatcaggaatgatatcagcaccaaaggttgagtctgtgaaaaccaccgcctctatgcctcgtccatcaggtattatatccgcaccaaaggctgagcttgtaaaatccgccgcttctgcagcctctttgccttgtacatcaggtattatatcttcaccaaaggctgagcttgtaaaatccgccgcttctgcagcctcttttcctcgcccatcaagtatgctatcagcaccaaaggctgacccagtaaagattgttcctgctgctgccactaacactaaatcggttggacctttgaatttaaggcatgcagtcaatggaagcccaaaccacacgataccttcatcaccctttactaagcctttacatatggctcctctctccaaaggatctacaatccagagtaattcagttcctcctagttttgcatcgtcaaggttggtccccacacagagagctcctgcggctactgttgtcacgccacaaaagccaagtgtggtagcggcagctactgttgtcacgccacaaaagccaagtgtgggagcagcagctactgttgtaacgccacaaaagccaagtgtgggagcagcagctaatgttgtaacgccacaaaagccaagtgtgggatcagcagctactgttgtaacgccacaaaagccaagtgtgggagcagcagttaccgtcacttccaagccggttggtgtacagaaagagcaaactcagggaaacagagcaagccccttggttacagcaacacttccgccaaataaaaccatcccagcaaattcagtgattggcacagcaaaagcggtggctgcgaaagtggagactcctcctagccttatgcctaagaaaaatgaagtagttggcagttgcaccgataaaagttcattggataaaccacctgagaaagaaagtactaccacggtgtcacctctagctgtagctgcgactaaatcaaaacccaaagatgaagcaaccgtgacagggaccggactgaaggagttgtag

SEQ ID NO: 45 - DNA, 3969 bp, Arabidopsis thaliana
atgggttataccttgcaacagatactgaggagcatctgctccaacacggattggaactacgccgtgttctggaaacttaatcaccactcgccaatggttcttactttggaggatgtgtactgtgttaatcatgagcgcggtttgatgccggaaagcttgcatggagggcgccatgctcatgaccctcttgggttagctgtggctaagatgtcatatcatgtacactctcttggggaagggattgtaggacaagtagcaatctctggacaacatcaatggatcttctctgaatatttgaatgactctcattcgacacttcaggttcacaacggttgggagagtcaaatttctgctggaattaagacaattcttatagtagctgttggttcttgcggagttgtgcagcttggctctttgtgtaaagttgaagaagacccggctttggtgactcatatcaggcatttatttttggcacttacggatccactagcagaccatgcatcaaatttaatgcaatgtgatattaacagtccatcggatcggccaaaaataccttccaaatgcttacatgaggcatcccctgatttctcaggagaatttgacaaagctatggatatggaagggttaaatattgtatctcaaaacacaagtaatagaagtaacgaccttccatacaatttcactccaacatattttcacatggagaggactgctcaagtaattggtgggcttgaagcagtccaaccttccatgtttggaagcaatgattgtgttacaagtggtttttcagttggtgtggttgatactaaacacaagaatcaagtggatataagtgatatgagtaaggtgatttatgatgaggaaacaggtggataccgatactcaagagaattagatcccaatttccaacactactcgaggaatcatgtgcgtaatagtggaggcacatctgctttagctatggagagtgataggctaaaagcaggttcatcatatccacaacttgattcaactgtacttactgcgttgaaaacagataaagattattctcgtcgaaatgaggttttccaaccatctgagagccaaggaagtatatttgtgaaagatacagaacataggcaggaggaaaaaagtgagtcaagtcagttggatgctttaactgcatctttgtgttctttttctggcagtgagctgttagaggcattagggccagcgttcagtaaaacaagcactgattatggggagctagcaaagtttgaatctgctgcagctataagacgaacaaatgatatgagccatagtcacctgacatttgaatccagctccgagaatcttctagatgccgttgttgctagtatgagtaatggtgatggtaatgtcaggcgtgaaatatcttcaagcaggtcaacacagtcattgcttacaactgctgaaatggcacaggcagaaccttttggtcataataagcaaaatattgttagcacagttgatagtgtgattagccagccgcctctagcagatgggcttatccaacagaatccatcaaatatctgcggagcattttcttccattgggttttcatcaacatgtctcagttcatccagcgaccagtttccgacgtccctggaaattcccaagaagaacaaaaagagagctaaacctggtgaaagttctcggcctcgtccaagggacaggcaacttattcaggatcgtatcaaagaactaagggagcttgtgcctaatggatctaagtgcagtattgattccttgctagagtgcacgatcaagcacatgctcttcctgcagagtgtctctcagcatgctgacaagctcactaaaagtgcaagttcaaagatgcaacacaaggataccggcaccctaggaatatcaagcactgaacaaggttcgagctgggcagtggagattggaggccatctgcaagtgtgctcaatcatggtggagaatctggacaaagaaggagtgatgcttattgagatgctatgcgaagaatgtagccactttctcgagatagcgaacgtgataaggagcttggaactcatcatcctcagaggcaccactgagaaacaaggcgagaaaacatggatatgttttgtagtggagggacaaaacaacaaagtaatgcacaggatggacatcctgtggtctcttgtgcaaatatttcaacccaaggctacaaacagtctgcatctttatcgacaatctcaaattctttacatgaatgctttcgccaatgtgcatagtcttcgggtaccttctcaccatcttcgagatttctcagcgtcactctctctggctcctccaaatttaaagaaaatcatcaagcaatgctcgacgcccaagcttctggagtctgctttagccgccatgatcaagacgagcctaaaccaagactgtcgcttaatgaaccaattcatcactgcctgcacttcctttaaacgtcttgacctcgcagtttccaccatgacccagatgcaggaacctaatgttttcgtctacaacgcgttgtttaaaggcttcgttacttgttctcacccgattcgatctctggaattgtatgttcgtatgctcagggactcggtttctccatcaagctacacgtactcttcactagtaaaggcgtcttctttcgcttctaggtttggggaatcactccaggcgcacatctggaaatttggatttggtttccatgttaaaattcagacgactcttattgatttttattcagccactggtagaatcagggaagccaggaaagtgtttgatgaaatgcctgaaagagatgatattgcttggaccacaatggtttctgcttatcgtcgggttttggatatggactctgcgaattctttagctaaccaaatgtcggagaagaatgaggctacgtcgaactgtttgattaatggatatatgggattaggcaatctggaacaagcagagtcattgtttaatcagatgcctgtgaaggacataatctcatggaccactatgatcaagggttactcgcagaataaaagatatagagaagcaattgcagtgttctacaaaatgatggaggagggcatcattcctgatgaggttactatgtcaactgttatttcagcttgtgcccatctcggcgtgctggaaataggtaaggaggttcatatgtacacgttacagaacggttttgttcttgatgtctacattggttctgcactggtagatatgtattccaaatgtggtagcttagagcgggcgcttctggtgttcttcaatttgcccaaaaagaatctattttgttggaattcgatcattgaaggactggcggctcatggttttgcacaagaagcactgaaaatgtttgccaagatggagatggagtcggtgaaacctaacgcagtcacttttgtgagtgtttttactgcgtgtactcacgcaggtcttgttgacgaaggtcggaggatatatcgcagcatgattgatgactattccattgtctctaatgttgaacattacggaggcatggttcatctattcagcaaagctgggttgatctatgaggctcttgaattgattggaaatatggaatttgaaccaaatgcggttatctggggggccttgcttgatgggtgcagaattcacaagaatctcgtgatagctgaaatagcgtttaacaaactgatggttttggagccgatgaatagtgggtattatttccttttagttagcatgtatgcagaacaaaacaggtggagagatgttgcagagattaggggaaggatgagagagttgggtatagaaaagatatgtcctgggacaagttcgattcggatagataaacgagaccatctgtttgctgcagctgataagtctcactcagcttcagatgaggtttgcttgctgcttgatgagatatatgatcagatgggattagctggatatgtgcaggaaactgagaatgtatattaa

SEQ ID NO: 46 - DNA, 1746 bp, Arabidopsis thaliana
atgggattcttcgatcttagcattccgtacaatgagccgccacgatcaggtggtaaggaaatcgccggcgggaaaaccttacgattaaagctcgccacgaaagccatggagctaggctatgttgggatcgcacataaccgttcgatcaaaggcgtaatgtctgacaaagactcttgtacgatccctcttctcactcttgggtctctaatcaaagtcgctccgcggttagcttcttctgtcggattccatcgcgatttactcggtgttccgcgaactactccgtttcggcagtacacgcgtctcacagttcatgtggagagtaatgctcagtgtcagagtttgaattctgggaatccgattctaaagagttatgatattattgctgttaggccgatgaatcagaacgctttcgattatgcctgtgagaaagctgaggttgatcttatttcgatagattttacggacaagatgttattccgattgaagcatcccatggttaaagctgctattcagcgagggatttactttgagattaagtactctgatatccttatggatgcacaaacgaggagacaagttatatcaaatgctaagttactggtggattggactagggggaagaatctaattatatcaagtggtgctccttcagtcacagaacttagaggtccaaatgatgtcataaatctcatgttcttacttggactctctgctgaaagagctagagctgccatttcaaaaaattgtaggaatatgatagccaaggttttaaagaaaaaacggtttcacaaagaagctgtcagggttgaattactttctgctggtgatacttttagcctcgaacagcctctgtctgaagattgcatgaaatgggatcgcctttcgagcggtgaaggtgacatgcttttggatgatcttgcaaaggcttttgatgccacaaatgttgtggcgcacaaatcctcgaaggcgattgatttcacctctgttcttgatggcttgccaaaacatggtttccgggttaaggatattgtaggaactgaatcagtgactcagccttctgcagctaaggtgattgacactcaggtgcacagtagtaatcaagtttctgaactacgtatggccacagcttcatctgatgataaccttcgggaaattgaaaccataagccaaattgacatgctgatgtctgaagatgacaataaggtggaacctactacaaatgtcctcaaagaagaagcatttgccctaaggaaatgcagtgccagccatggccaggggattttggtgcaaaatcagacggctactccctttacactgacaagatgtacaaagtcagaagcagcgtcggatgttagcatgaatattgagtcgacttccgaaggtggatcaatgtcaccgtcaaaaagcgatcatgggatcccacaaagtcctgttgaagtgaataacatgggaaatgctgcttttgaagaagaagcctcagtggacgaaaacagcaaagaaagagctactactggtcatgctagtaatgatgagatgcatatcactgagtctggacaccacgcatccattgatgatgagaagcatatccctgagcctgaacacctcacatccattgctgatgagatgaaaattgattgttcttcggaagcaaatcacgacgagtacatggaggtcacaatggaagaccagatgcatgaaacagtccagatgcggttgtgcaagaccatgacgaagcatcaagactag

SEQ ID NO: 47 - DNA, 612 bp, Arabidopsis thaliana
atggcaaaaggtcgaaagccgacgacaatgaaccggagcgatcgataccttggaagctacacttacggtgacagtcacggaaactccgttaccgacgaattagagctcggtgaggaagacatctggtcaccggccgtcattcacgacgacaccaccgagaatgaggaatcctacggcacgtggaacttacgcgctaccttgggaaaaaacgggcgcgtgggaggattgtcgctggctttcgagggctctttggttgctccgccgtcgtcttcgccgatgatagtgcagaagattcacggcggaggaggtgagggagaggaagaccggagaaaattggcgtcttcggcgccggtaaacgtaccggactggagtaagatataccgagttgactcggttgagtcaatacacgagttagacgacgaggatgacgaggatgaggaatccgggatgatgccgccgcatgagtaccttgctaagagtcaagcacggcggagtagaaagatcggaggtggtggtgcgtcggtgtttgacggcgtcggaaggactctcaaaggcagagaactaaggcgcgttcgtgacgcgatttggagccaaacagggttctacggctaa

SEQ ID NO: 48 - DNA, 531 bp, Arabidopsis thaliana
atggatcaacaacaacaaggtgataagaatctgacagtgttcgtaggaccctggggaggaaatggaggaaccacttgggatgatgggatttatgatggtgtccgtgagatcagacttgtttatgaccattgcattgactccatctcggtgatctacgataagaatggtaaacccgcaaagtcagagaagcatggaggtgtgggaggcaacaaaacatcagagataaagctgcaatacccagaggagtatctgactggcgtgagtggctactactgtccaatggttaacagtggcactcctgtaatcagatcaatgaccttcaagagcaataaacaagtgtatggaccttatggagttgaacagggaacacccttcactttctcagtcaatgggggacgcattgttggtatgaacggtaggagtggctggtaccttgactccatcggcttccatctatcacgccctaaatcaaccaagatgatcaacaagctccgaaagaagattcactggctcacaaggatagtagcatga

SEQ ID NO: 49 - DNA, 384 bp, Arabidopsis thaliana
atgactaatataggaaaatgcatgcagggatatctcgacgaacaattcatggagttagaagagctccaagatgatgcaaaccctaattttgttgaagaagtttccgcattatacttcaaagattcagctcggttaatcaataacattgaccaagctttggaaagaggatcatttgatttcaatcggctggatagttacatgcatcagtttaagggaagcagcacgagcattggggcaagtaaagtgaaagctgaatgcactacgtttagggaatactgcagagctggaaatgcggaaggatgcttgaggactttccagcaactgaagaaagaacactcaacgttgagaaagaagcttgaacattatttccaggcgagccaataa

SEQ ID NO: 50 - DNA, 1575 bp, Arabidopsis thaliana
atgcctctgtttgagcttttcaggctcaccaaagctaagcttgaatctgctcaagacaggaacccttctccacctgtagatgaagttgtggagctggtgtgggaaaatggtcagatatcaactcaaagtcagtcaagtagatcgaggaacattcctccaccacaagcaaactcttctagagctagagagattggaaatggctcaaagacgactatggtggacgagatccctatgtcagtgccatcactaatgacgggtttgagtcaagacgatgactttgttccatggttgaatcatcatccctcccttgatggatattgctctgatttcttgcgtgatgtgtcgtctcctgttactgtcaacgagcaagagagtgatatggcggtaaaccaaactgctttcccgttgtttcagagaagaaaggatggcaatgaatcagctcctgctgcttcttcgtcgcagtataacggtttccaatcgcattctctgtatggaagtgatagagctagagatcttcctagccaacaaaccaatccggatcggtttactcagacgcaggaaccactaattactagtaacaagcctagtttggtcaacttttcacatttcttacgccctgcaacttttgcgaagactactaataataaccttcatgacactaaagaaaagagtcctcaaagcccgccaaatgtgtttcagaccagagttcttggagctaaagactctgaagataaggttcttaacgagtctgttgcttctgctacgcctaaagataaccaaaaggcttgcctaatatcagaggactcatgtagaaaagaccaagagagtgaaaaagcagttgtatgttcttctgttggctcgggtaatagtctcgatggcccatccgaaagtccttcactttctttaaagagaaagcattcgaatattcaagacattgactgtcatagtgaagatgtggaagaagaatcaggagatggaagaaaggaagcaggtccatctcgaacgggtttgggttcaaagagaagccgctctgcagaagtgcataatctgtctgaaaggagacggcgtgataggatcaacgagaagatgcgtgccctgcaagaactcattccaaactgtaacaaggtggacaaagcttcgatgctagatgaagccatcgagtatctcaagtcactccaacttcaagtgcagatcatgtcaatggcgtctggttactatctgccaccggcggttatgttcccaccgggtatggggcattacccggcagcagctgctgcaatggcaatgggtatgggaatgccttatgcaatgggcttgcctgatttgagccgtggtggttcatcggttaaccacggaccacagttccaagtctcggggatgcaacaacaaccagtggcgatgggtattccacgtgtctctggtggtggtatctttgccggttcttcgacgattggcaatggctcgactagagatttatctggttctaaagatcaaacaacgacgaataacaacagtaacttgaaaccaataaagagaaaacaggggtcttctgatcagttttgtggatcgtcgtga

SEQ ID NO: 51 - DNA, 657 bp, Arabidopsis thaliana
atggagaatgattgcacggtgaatattgtctctctggagaaggatcgcgatgtttcggaggcgtcggctgaatctcagagcgagtcgactctttcgaactcgctcgattccggtgttacggctgagacctctcgttctgatgctgattccaaactggatgaatgtactgcttggacgaatgagaaacacaactcatatcttgattatttagagagctcgtttgttaggcaattatactccttgcttggaggtgggactcagagactttctagaactcgtgatgtgcagtctaactctcataaatcagctgatcagtttaccgtcctacaaaatggttgctggcagaaggttaactttggaaagaaacaatcttgtttggagacttcatctgagtttcgttttcacagaaattcattgagaaataagcctgaaaattccaacggaaattacaccatgggaactactgtccaaggagatgtgttatgtcatgacgaaaccaaacactcagaggcgtcagggcagaatttcagagaagaagaagaagaagaagagaagggagaggtgagcaaaaaacgagaaagagaagcaaataacgatgatagttcattgaaggaggatcaggttgtgccggtaaggatggtgaagcccagaacgtga

SEQ ID NO: 52 - DNA, 459 bp, Arabidopsis thaliana
atggctgaaaaagtaaagtctggtcaagtttttaacctattatgcatattctcgatctttttcttcctctttgtgttatcagtgaatgtttcggctgatgtcgattctgagagagcggtgccatctgaagataaaacgacgactgtttggctaactaaaatcaaacggtccggtaaaaattattgggctaaagttagagagactttggatcgtggacagtcccacttctttcctccgaacacatattttaccggaaagaatgatgcgccgatgggagccggtgaaaatatgaaagaggcggcgacgaggagctttgagcatagcaaagcgacggtggaggaagctgctagatcagcggcagaagtggtgagtgatacggcggaagctgtgaaagaaaaggtgaagaggagcgtttccggtggagtgacgcagccgtcggagggatctgaggagctataa

SEQ ID NO: 53 - DNA, 1017 bp, Arabidopsis thaliana
atggcggctccgcatttcacacaactcaaaattacactaaaccctctcatgtatccctttctcgtcttatctctactaactctcgccctcttctcattcgtctccgccatcttctttctcctcaaagcttcccgcagcagagctgctttgtacagccagaaactcttatccgaatccgaaaccaaactccaaccagaatcgtctctatcggagatttccgacgaagcccagtaccaaacccatgaaaatgaaccgacccatttgacgaattcgcgactctatgagttactgctctccgataagaaggaggatgattcggattgggaaggagatcatgtgaaaaagaagaagaagaagaagaagaatcgaggtaagaagaagaaatcagacataagaggagatgaatccggcggcgaaaagcagctcggtgagggagaagatgggcttgttttgaatccgaggacagactcgatttcgatatcggaaaacaaaccggagtttgtttgtttatatccttttacatcgacgagcagtgctacgcagaggaagattaagcagcaatacgatcagcttgttaaatgcaataatgccaaaggattgacactagctcaggttggggagtttgctaattgtttgatagaagccaaaaatgaactacaacacaagtcagaagtaatcaagcgcaagttttcaataacaaaagcccttctctttaaggctgatagatcttcctttgaccgacttcgtcaacagatctataagctggagatggaacaaaaaagagtagaagaagatgcacttgtatataattggctccagcaacagcttaaactctcacctgcatacaaaaaggttcttgaaataagcgcttccatggaactcaaagacaaatcgagcacagagttagacaatccagatgatgaattttcagacatttccttcgaagagctattggaacaggaaaagaaagactcgttttggtcagcatttctctccatctctcctcaagcttattag

SEQ ID NO: 54 - DNA, 1302 bp, Arabidopsis thaliana
atgagtagttcggagagagtaccgtgcgatttctgcggcgagcgtacggcggttttgttttgtagagccgatacggcgaagctgtgtttgccttgtgatcagcaagttcacacggcgaatctgttgtcgaggaagcacgtgcgatctcagatctgcgataattgcggtaacgagccagtctctgttcggtgtttcaccgataatctgattttgtgtcaggagtgtgattgggatgttcacggaagttgttcagtttccgatgctcatgttcgatccgccgtggaaggtttttccggttgtccatcggcgttggagcttgctgctttatggggacttgatttggagcaagggaggaaagatgaagagaatcaagttccgatgatggcgatgatgatggataatttcgggatgcagttggattcttgggttttgggatctaatgaattgattgttcccagcgatacgacgtttaagaagcgtggatcttgtggatctagttgtgggaggtataagcaggtattgtgtaagcagcttgaggagttgcttaagagtggtgttgtcggtggtgatggcgatgatggtgatcgtgaccgtgattgtgaccgtgagggtgcttgtgatggagatggagatggagaagcaggagaggggcttatggttccggagatgtcagagagattgaaatggtcaagagatgttgaggagatcaatggtggcggaggaggaggagttaaccagcagtggaatgctactactactaatcctagtggtggccagagttctcagatatgggattttaacttgggacagtcacggggacctgaggatacgagtcgagtggaagctgcatatgtagggaaaggtgctgcttcttcattcacaatcaacaattttgttgaccatatgaatgaaacttgttccactaatgtgaaaggtgtcaaagagattaaaaaggatgactacaagcgatcaacttcaggccaggtacaaccaacaaaatctgagagcaacaatcgtccaattacctttggctctgagaaaggttcgaactcctccagtgacttgcatttcacagagcatattgctggaactagttgtaagaccacaagactagttgcaactaaggctgatctggagcggctggctcagaacagaggagatgcaatgcagcgttacaaggaaaagaggaagacacggagatatgataagaccataaggtatgaatcgaggaaggcaagagctgacactaggttgcgtgtcagaggcagatttgtgaaagctagtgaagctccttacccttaa

SEQ ID NO: 55 - DNA, 1929 bp, Arabidopsis thaliana
atgcctaatttctcagttaacgttccccaactctcatctctttacagtacaaaaacgcccaaagtgagaatgaatctatgtgccgatcaggtgttcgataaaaagcttctgtggagagatatgtcaacgaagatgaaatttccttctttttctgctgcggaattacctgatttgaggaaaagtaacaagaggaggggatctcttaggatgatcaagtgcagagccgccggagctgacggtggacgcgtggctgttggggatgatgtgttttcggttactacttcttctaagtatgaagttgactatctgggtcaaagtactaagggagatttgaatctcaagcttgaccctcttcagtcatttggagatgggcaggctacattggagggtcccattgaggaggtagcgagaacagaggctcaagcggctgaaaatttgattagagagttgggtatccaaggccctttctctgcacagcactctcctcggggtatattttgtagtcgtacattgaatcttcggtccattagtgcaattggatatgatatggattacactttgatgcactacaatgtcatggcttgggaaggaaaggcttatgactattgcatggaaaatctaaagagcatgggtttccctgttgatggacttgcttttgatccggaactggttatcaggggtctcatgattgacaaagagaaaggtaatttagttaaggccgatagatttgggtatgtgaagagagccatgcacggtacaaagatgttatcaaataaagctgtcagtgagatctatggaagggagttagttgacctgcggaaccagagtcgatgggagtttctcaatacatttttttcagtttcagaggctctggcttatgcacagatggttgatagattggatgatggatttatttcggcagatcttggcactcttgattataaaggactgtataaggctgttgcaaaagctctcttcagagcacatgttgaaggacaacttaagagtgagataatgtccaagccggaactatttgtcgagccagacccagaactacctttagctcttttagatcaaaaggaggctggtaagaagctcttgcttatcacaaactcggattatcactacacagacaaaatgatgaagcattcatttaacaaattccttcccaatgacatggactggcgagatctttttgacatggtgatagtttctgcgaggaaaccagagttcttccagatgtcgcaccctctatatgaggttgtgactggagagggtttgatgcgtccatgcttcaaggctgaaacaggaggtttgtactcaggaggaagtgctcaaatgatagagagttcactcaacgttcatggagatgagattttgtatgttggtgaccacatctacactgatgtcagcgtatccaaagtccatctcaggtggcgaactgcgctgatttgccgtgaactggaagaagagtatatggctctaattggcagtcgtggtcaccgagaagagctaatagagcttataaatcaaaaagaggttgttggggatctctttaaccaacttcggcttgctcttcaaagacgaagcaaaggccgtcctgctcagactctcgctgctaccaacttggatgatcaagaactgacagagaccatgcaaaagcttcttattgtaatgcaaagactagatgacaagattggtctaatgctggaaacagatggcgagctctttaacaaaaggtggggcttcctctcacgcgcgggtttgtgggataaaagccacttgatgagacaaatcgaaaagtatgcggatatatacacatcaagagtctccaacttcctcaactacacacccttcatgtatttccgctcacaagagcagtcactggctcacgattctccgcttccagatgcgggtatagaaaactag

SEQ ID NO: 56 - DNA, 321 bp, Arabidopsis thaliana
atgtgggatgaaactgtagccggacctaaaccggagcatggccttggccgcctccgcaataagatcaccacccaaccccttgacatcaaaggagaagggagcagtagtaaaactgtggcggcggtggccgggagtcctggaactccgacgacgccaggatcggcgcgtaaggaaaacgtgtggagaagtgtgtttcatccaggaagtaacatcgccactagaggaatgggcacaaacctcttcgacaagccttctcacccaaactctcccaccgtctacgattggctatacagcgacgacactaggagcaagcaccgttga

SEQ ID NO: 57 - DNA, 2106 bp, Arabidopsis thaliana
atggagattccactctcgcgttaccagagcataagattagacgagattcgagactcttcttccaatcccaaggttctcactttcccgcgaaaattctcgttacgaggaagaagatggaagaacccatttggaagactcagttgttcttctgtagttcaaggtctgaaaccaaaaccaaagctgaaaccagaaccaattagaatcgaggttaaggaatcgaaagatcagattttggatgatacccagatcagtaaatctggtgtaacgatttgtagtcagatagagaagttggttttgtgtaatagattcagagaagcttttgaattgtttgagattctggagattcgctgtagttttaaggttggtgttagtacttatgatgctttagtggaagcttgtattcgtttgaaatcgattcggtgtgttaaaagggtttatgggtttatgatgagtaatgggtttgagccggagcagtatatgatgaacagaatcttgttgatgcatgtcaagtgtgggatgattattgatgcacgtaggttgtttgatgaaatccctgagagaaatttgtattcttattactcgattatctctgggtttgttaattttgggaattatgttgaagcttttgagttgtttaagatgatgtgggaggagctttctgattgtgagactcatacgtttgcggtgatgctacgggcctcggctggcttagggtctatttatgtggggaaacagttacacgtttgtgcgttgaagttaggagttgttgataatacctttgtctcgtgtggattgattgatatgtatagcaagtgtggggatattgaagatgctcgatgtgcttttgagtgtatgcccgagaaaactactgttgcttggaacaacgttattgcgggttatgcgcttcatggttatagtgaggaagctctgtgtttgttgtatgacatgcgagactcaggtgtgtctattgatcagttcacactttcgataatgataagaatttctacaaagcttgcaaagcttgagcttactaagcaggcacacgctagtttaattcgaaacggttttgaatcggaaatcgttgcaaacacagctcttgtagacttttatagcaaatggggtagagtagatactgctagatatgtttttgataaattgccgagaaaaaatataatctcatggaacgctttgatgggtggatatgcaaatcatggtagaggaactgatgctgttaagttgtttgagaaaatgattgcagcaaacgtcgctccaaaccatgtcacatttcttgcagttctatcagcttgtgcctattcaggtttatctgagcaaggttgggagatttttctatcgatgagtgaggttcatgggatcaaaccaagggcgatgcattatgcctgcatgattgagctgttgggtagagacggtttattagatgaagccattgcgtttatccgaagagctcctttgaaaaccacggtgaacatgtgggcagcactcttgaatgcctgtaggatgcaggaaaacttagagcttggaagagtggttgctgaaaaactctatggaatgggacccgagaagctcggaaactacgttgtgatgtataatatgtacaacagtatgggaaaaactgcagaagctgcaggggttttggagacattggagagcaaaggattaagcatgatgccggcttgtacttgggttgaggttggagatcagactcacagctttctttcaggagataggtttgattcttacaatgagacggtgaaaaggcagatataccaaaaagtggatgaactaatggaagagatttccgagtatgggtactcagaggaggagcaacaccttcttccagacgtagatgaaaaggaagaagagcgagtagggcgatatcacagcgagaaactagccatagcttacggattggtgaatacgccggaatggaatccattgcagattactcagaaccataggatatgcaaaaattgccacaaggtggttgagttcatatctttggttacaggacgagagatggtagtgagagacgcgagccggttccatcattttaaagaagggaagtgttcttgtggaggttattggtga

SEQ ID NO: 58 - DNA, 1500 bp, Arabidopsis thaliana
atgaaaacttgtttgatcttcttcctctacacaacaattctccaatactatttccacttctctgtgtcatcattatcaacacctcttctcctccatctctcccactctctctcaacctcaaaacactcttcatctcctctccaccttctcaaatcatcctcctcccgttcctccgcccgcttccgccgccaccaccacaaacaacaacaacaacaactttcactccctatctcctccggcagcgattatctcatctccctctccgtcggctcctcctcctcagccgtctccttgtacttggacaccggaagcgacctcgtttggttcccttgccgtcctttcacttgcatcctctgtgaatccaaaccactccctccttctcctccttcatctctctcctcctccgccaccaccgtctcctgctcctccccttcttgctccgccgctcactcttcccttccctcctccgacctctgcgctatctccaactgtcctcttgatttcatcgaaaccggagattgcaacacttcttcttacccttgtcctcctttctactacgcttacggtgacggctctctcgtcgcaaaactctactccgactcactctctctcccttccgtctccgtctctaacttcaccttcggctgcgctcacaccactctcgctgaacctatcggcgtcgctggattcggccgtggacgtctctctcttcccgctcagctcgctgttcactctcctcatctcggtaatagcttctcttattgtctcgtctctcactcttttgactcggaccgagtccgccgtccgagtccgctcatcctcggtcgcttcgtcgataaaaaagagaaacgtgtcggaaccaccgatgatcatgatgacggtgatgatgagaagaagaagaaaaatgagttcgtcttcacagagatgcttgaaaacccaaagcatccctacttctactctgtttcactccaaggaatctcaatcggaaaacggaatattccagctccggcgatgctcagaagaattgacaaaaacggtggcggaggagttgttgttgactcagggacaacgttcacgatgcttccggcgaaattctacaattcggtggttgaagaattcgatagtcgggtcgggcgggttcacgaacgggctgatcgggtcgaaccgagttcgggtatgagtccttgttactatttaaaccagacggttaaagttccagctctggttttgcattttgccgggaacagatccagtgtgacgctccccaggagaaattatttttacgaatttatggacggtggagatggtaaagaagagaagaggaaaattggatgtttgatgttgatgaacggtggagatgaatcagaacttcgaggtggtactggggctattctggggaattaccagcaacaagggtttgaggtggtttatgatctgttgaacagaagagttgggtttgctaagaggaagtgcgcatctttatgggattcgcttaaataa

SEQ ID NO: 59 - DNA, 1284 bp, Arabidopsis thaliana
atggcattaacccttttgtctcacgaattatctgacctctgtatcggtaagccacctttacggtgtctctccgtcgccacagccaccgtagctgacgccatcgccgctctcaaatcctctgacgaaccgttcctcaccgtatggagctgtaatcacgatgagaaaacagatgataatgataagtgtgagtgtttgggtaagatctgtatggctgatgtaatctgttacctatccaaattcgacaacaatgttttgtctctttcctctgctttcgacgcatctgtctctgttcttcttcccaaatctcgtgccctcgtcgtccatgttcaatcttcttgcagtttgattgaagctattgatctgataatcaaaggagcacagaatctgattgttccgattcatacgaaatcaatcacaaagagaagacaacaacaaaaacttctgaaacgaaacgtcgtcgtttcactcaccaacgcaacttcaacaacccacaaaaacagccgagaattctgctggatcacacaagaagacattattcgattccttctcgattccattagcgttttctctccattaccgtcgctttctatctccgatcttggagttatcaatagtacacacactatcctcgccgttgattactactcctcagctgcttccgccgtctctgccatctctcgtgccatcttggacaatgtctctgtcgcggtggttggtaaaggatgtgatcaagaagatccatgtatggttttgataggcgagatttcaccgatgacactcgcttgctgcgatgaaactgccgtagcagcggttgctacactctctgccggagatttaatgtcctatatcgacggtagtggtccgccggagagtctagttggagtagttaggaatcgtttggaagataaagggatggttggattaatctcactcattgattctttgtcgttgtcgtcggggtcttcctcggatgaagaatctccggcggggaagacgagaatgacttcttcgtatgggaggtcggtgagtagcgcggcgaggatggctaggaaatcggtggcgatagtgtgtaatcggaagagttctttaatggcggtgatgatacaagctattgctcatagagtgagttatgtgtgggtgattgatgaagatggttgtttgattggttgtacaatcggaatcagtaattatgtaaataggttagttagaccacgtattttggctgcaatatgggtttcacgtctaaacctaaagaaagcaacacttgatgagtctcacgtctaa

SEQ ID NO: 60 - DNA, 1098 bp, Arabidopsis thaliana
atggcatctgcattttgctcactttgtcccactcccacctccttattctcttcccacgcgcttatacccactctacagtggcgttcgagttcgagttcgaggtctcctccgctacatatttcccgcgttttatcagttgaaactgttcctttaagcccatcattcacctggaacgatgtttttgagaacagtcgaaaagaatacgtgcctcagaactcctccgatctcaccggatttctcgagaaagtcgaccgctgtaatcgtggattagagaagttaggtgagttcattccatttgttatagaggaacaaatcgttggttatattcacaagggatttacaaagtacttgagggactttaatgatatctttacattttcacaatatggtggccatgtaacgcttaacatgatgcttgacaagcctgaagaaagaaccagagcagttgcacatgtgatcaaaatattgggtaacaaaggaatcatccctgggatacgaaatgagctatatcctgtgaagccatcgtttaatgctcctgcctttttttctatagagcgtgctgctgctccttattttggattgaagggttacgcaattcatgtgaatgggtatgtagaaagagatggacaaaaatttctatggataggtaaaagaagtctagcaaaatccacttatccaggaaaacttgatcatctggttgctggaggattgcctcacgggattagcgtttgtgagaatctagtaaaggaatgcgaagaggaagctgggatttccaaagtcttggctgatagggcgattgcggtcggtgttgtttcctacatggatatcgatcggtactgtttcacacgtgatgtgctgttttgttatgatttggaactccctcaagattttgttcccacaaatcaagatggagaagttgacagcttcaggttgattccagtcgctcaagttgctaatgtggttcggaagactagtttttttaaggacagttgttccctggtcattattgacttcttgtttcggcacgggttaatcagaccagagtcaccgggttacttggatctataccgacgcctgaggaatggagattgctcataa

SEQ ID NO: 61 - DNA, 2268 bp, Arabidopsis thaliana
atggaaatctacaccatgaaaacgaattttcttgtactggctttgtctttgtgtatccttctttcaagcttccatgaggtttcttgtcaggatgatggtagtggtttgagtaatttggatctaatagaacgtgattatcaagatagtgtcaatgctcttcaaggcaaggacgatgaagatcagtctgcaaagatacagagtgaaaaccagaataacactacagtgactgataagaacactatttctctatctctatcagatgaatctgaggttggatctgttagtgatgaaagcgttggacgttcgagtctgttggatcaaatcaaacttgaattcgaagctcatcacaatagtattaaccaagctggatctgatggtgtcaaggctgaatccaaggatgatgatgaagaattatctgctcatagacagaaaatgttggaagaaatcgaacatgagtttgaagctgcttcagatagtctgaaacaactaaagactgatgatgtaaacgaaggaaatgatgaagaacattctgcaaagaggcaaagtttgttggaagagatcgaacgtgagtttgaagctgctacaaaagaacttgaacaactaaaggttaatgacttcaccggggacaaagatgacgaagaacactctgcaaagagaaaaagtatgcttgaagctattgaacgcgagtttgaagctgctatggaaggcattgaagcacttaaggtttctgattccacaggaagcggagatgatgaagaacaatctgcaaagagactaagtatgcttgaagagatcgaacgggaatttgaaggtcttgaacaactaagggctagcgattcaaccgcggacaataacgaagaagaacacgctgcaaagggacaaagtttgttagaagagatcgaacgagagttcgaagctgctacagagagccttaagcaacttcaagttgatgattctactgaagacaaagaacactttacagctgcaaagaggcaaagtctgctggaagagattgaacgtgaatttgaagctgcaacaaaagatcttaaacaactaaatgatttcactgaaggcagtgctgatgatgaacaatctgcaaagagaaacaaaatgttggaagatatcgaacgcgaatttgaagctgctacaataggtcttgaacaactaaaggctaatgatttctctgaaggcaataataatgaagaacaatctgcaaagagaaagagtatgcttgaagagatcgaacgcgagttcgaagctgctattggaggtcttaaacagatcaaagttgatgattccagaaatcttgaagaagaatctgctaagagaaagataattttggaagagatggaacgtgaatttgaagaagcacacagtggtattaatgcaaaggctgacaaagaagaatctgcaaagaaacagagtggctctgctataccagaggttcttggactaggacagtcaggtggttgtagctgttctaaacaagacgaagattcctcgattgttataccaacaaaatatagcatagaagatatcctctctgaagaatctgcagtccagggaacagagacttctagtctcaccgcgtctttgactcaactcgttgagaatcacaggaaagaaaaggaatctctactcggacacagagttctcacttctccttctatagcttcttccacaagcgaatcatctgctacatcagagactgtagaaaccctaagggctaaactgaatgagcttcgcggcttaaccgctcgtgagcttgtgacacgtaaagatttcggtcagattctcattacggctgcgagttttgaagagctaagttcagctccaatcagttacatttctaggttagctaaatacagaaacgtcatcaaagaaggacttgaagcttctgagagagttcacatcgcgcaggtacgagcaaaaatgctcaaagaagttgccacggagaagcaaaccgccgtggacactcatttcgcaaccgctaaaaagcttgctcaagaaggagacgcgttgttcgttaaaatcttcgcaatcaagaaactgttggcgaaacttgaagcagagaaagaatctgttgatggaaagtttaaggagactgtgaaagaactttctcatcttctggctgatgcttctgaggcttacgaagagtatcatggcgcggtgaggaaggcgaaagacgagcaagcggctgaggaatttgcgaaagaggcgacgcaaagtgcagagatcatttgggttaagtttcttagttctctttag

SEQ ID NO: 62 - DNA, 501 bp, Arabidopsis thaliana
atggcagcgcgtgcccatgatgttgcggcattgagtatcaaaggaagttccgcaatccttaacttccctgagctcgcggattttctgccaagaccagtctcgctcagccaacaggatatccaggccgcagccgccgaagccgctcttatggatttcaaaactgtaccattccatcttcaggatgactcaacgccgttgcaaactaggtgtgatactgagaagatcgaaaagtggtcatcctcatcgtcctcagcctcatcctcatcctcatcttcgtcctcgtcctcatcatctatgctttcgggggagctaggagatattgtggagttgccgagtcttgaaaacaatgtaaaatacgattgtgcgctgtatgactcgttggaggggctggtgtcgatgcccccatggttagatgctaccgaaaatgattttaggtatggagatgattcggtactgttggacccatgtctcaaagaaagctttttgtggaattatgagtaa

SEQ ID NO: 63 - DNA, 465 bp, Arabidopsis thaliana
atgtcgctagacgcgaatacttgttctatcgtcttctttctattcttcactgtctctttcgccgtctccggccaaaagccaaccgcttacgacgccgtcaaactctataacttaccaccaggaatcctcccaaaaggagtggttgactatgagcttaacccaaaaacaggcaacttcaaagtctacttcaatgacacgtgtgaattcaccatccaatcctaccagctcaagtacaaatcaactatctccggcgttatatcacccggtcatgtcaagaatctgaaaggagttagtgttaaggttcttttcttctgggttaatattgctgaagtgtctcttgacggcgccgatctcgacttctccgtcggaatcgcgtcggcgagttttccggctgctaattttgaagagagtcctcagtgtggttgtgggtttgattgtaataatgggcttttattttcttcttga

SEQ ID NO: 64 - DNA, 891 bp, Arabidopsis thaliana
atggctcaaaaggttgaagcaaaaggagggaaaggaggcaaccaatgggacgatggatccgatcatgacgccgtgaccaagattcaggttgcagtaggcggaatgggaattcaatacattcagttcgattacgtcaagaacggacaaaccgaacaaactcctcttcgtggtatcaaaggcagtaccattccaactgatccgtttgtgattaaccatcctgaggagcatctagtttctattgaaatttggtataaacctgatggtcttattcaagggcttaggttcatatccaacaaaaagacttctcgtttcattggatacgatcgtggtactagatcatttctccaagttcaagacaagaagatcattggctttcatgggtctgccggagacaatcttaattctcttggagcttactttgctccgttgactatcccattgactcctgccaagccgctaccggcacttggtagtgatgatggaacagcatgggatgatggtgcttacgttggggttaagaaggtgtacgtaggacaagcccaagatggtatatcggctgttaagtttgtatacgacaaaagccctgaggaggtcacaggagaagaacatggaaagagtactctactcggattcgaagagttcgtacttgactatccaagtgaatacatcatcgcagtcgaaggcacctacgataaaatctttgggagtgatggctcagtcataactatgcttaggttcaagactaataagcaaacgtcccctccctttggacttgaagctggcactgccttcgaactcaaagaggaaggccacaaaatcgttggcttccatggaagagccgatgctttgctccacaaaattggagttcatgtccgtcccgtttccaactga

SEQ ID NO: 65 - DNA, 1071 bp, Arabidopsis thaliana
atgtcttcacttgcagatttaatcaatctcgatctctccgattccactgaccagatcatcgccgagtacatatggattggtggatcgggcttggatatgagaagcaaagcaaggactttgcctggaccagtgacggatccatcgcagttaccgaaatggaactacgacggttcaagcaccggccaagctccgggcgatgacagtgaagtcatcatctaccctcaagctatcttcaaagaccccttcagaagaggcaacaacatccttgtgatgtgtgacgcatatacaccggcaggagagccgattccgacgaacaaaaggcatgcggcggctaagatctttgaagaccctagtgttgtcgccgaagaaacatggtacggaattgaacaagagtataccttgttgcaaaaggatattaagtggccggtaggttggccggtcggcggtttcccaggtcctcagggaccgtactactgtggagttggagcagacaaagcctttggaagagacatcgttgattctcattacaaagcttgtctttacgccggaatcaatgtcagtgggactaacggcgaagttatgcctggccagtgggagttccaagtcggtcccaccgttggaatcgctgccgccgatcaggtctgggttgctcgttacattcttgagaggatcacagaattggctggagttgttctgtctctagaccctaaaccaattccgggagattggaatggtgcaggggcacacacaaattacagtacgaagtcgatgagagaagatggagggtacgaggtgataaagaaagcaatagagaagcttggattgcgtcacaaggaacacattgctgcttatggtgaaggcaacgagcgtcgtctcaccggaaaacatgaaaccgccgatatcaacactttcttatggggtgtggcaaaccgtggggcatcgattagggttgggcgtgacactgagcaggctggaaaaggatactttgaagatcgtaggccagcttcgaacatggatccttacactgtgacctccatgattgctgaatccacaatcctttggaaaccatga

SEQ ID NO: 66 - DNA, 990 bp, Arabidopsis thaliana
atgactgattctgcttacagagtagacaccatttctagactcgcccaatggcgaatccacaatctttcttcctccacttaccgcaagtccgatcctttcaagatgggtctttggaattggcacttgtctgtggagaaaagcaagatgctattaaatgttaagttgtatccagaggtatcaaaccttaccagagaaaatccaccggttgcttcctttgctcttcgtgttgtctcttctactggtgagagaaaggctctatctcatccagaagtaatagataagcggattaagacaaacgaagattttatttggactattgaagttcccttaactgggaaaatcatcatcgacgtcgagtttcttgacttgaaggttttgtctcaagatagtggagaactttactctatctgggccaacggttcaactgagaatcaatcgcaagtaactgcggtaacatcccttggacgtatgttgacagaaagcatttacaccgacataacgatcaatgcctctgatggaagcattggagctcaccgagcagttctcgctgcccgttcacctgttttccgcagcatgtttttacacgacctgaaagagaaagaactatcagaaataaacgtactcgacatgccacttgatgcttgccaagcttttctcagttatatctacggcaatatccaaaacgaagactttcttatacacagattggcactcctccaagcagctgagaaatatgatattgctgatttaaaagaggcgtgccacttgagtcttctagacgatatcgacacaaagaatgtgcttgagaggctacagaatgcttatctctatcaattacctgaactgaaggctagctgcatgagatatcttgtgaagtttggcaaaatatttgagatccgagacgagttcaacatattcatgcaatgcgcagacagagatttgatttctgaaatcttccacgaagtcctcagtacctggaaaggattttag

SEQ ID NO: 67 - DNA, 1113 bp, Arabidopsis thaliana
atgtcggcgatcatgaaaagtctctgtttctccttccttatcctcgcttcatttgcaactttcttctccgttgctgatgcatggaggtttaacgtcggaggtaacggcgcttgggttacaaatcctcaagaaaactacaatacttgggctgaaagaaaccgtttccaagtcaatgactctctctattttaagtacgcgaagggatcagactctgttcaacaagtgatgaaagcagattttgatggatgcaacgttagaaatccgatcaagaacttcgaaaatggtgaatctgtggttactcttgatcgatctggtgctttttatttcataagcggtaatcaagatcactgtcaaaagggacagaaattgatcgtcgttgtcctcgccgtcagaaatcaaccttcggctccggctcattcccctgttccttcagtttctcctactcaacctcctaaatctcattcccctgtttctcccgttgctccagcgtctgctccttcaaaatctcagccacctagatcctctgtttctccagcacaaccacctaaatcttcttcccctatttctcacacaccagctctctcaccgtcgcatgctacatctcactctccagctactccatctccgtcaccaaaatctccctcccctgtttctcactcaccatctcactctccggcacataccccatctcactctccggcacataccccatctcactctccggcacatgccccatctcactccccggcgcatgctccatctcactctccggcgcatgctccatctcactccccggcgcattctccatctcactcaccggcgactccaaaatccccatctccttcttcttctccagctcagtctccggccactccatctccgatgacaccacaatccccatcccctgtttcttccccatcacctgatcagtctgctgctccctctgaccagtctacacccttggcaccttctccttccgaaacaactccgaccgccgataacatcactgcgccggctcctagtcccaggacaaactcagcaagtggtttagccgttacttcggttatgtctacactatttagtgcgacttttacctttctgatgtttgcttaa

SEQ ID NO: 68 - DNA, 588 bp, Arabidopsis thaliana
atggctttgaagacagttttcgtagcttttatgattctccttgccatctattcgcaaacgacgtttggggacgatgtgaagtgcgagaatctggatgaaaacacgtgtgccttcgcggtctcgtccactggaaaacgttgcgttttggagaagagcatgaagaggagcgggatcgaggtgtacacatgtcgatcatcggagatagaagctaacaaggtcacaaacattattgaatcggacgagtgcattaaagcgtgtggtctagaccggaaagctttaggtatatcttcggacgcattgttggaatctcagttcacacataaactctgctcggttaaatgcttaaaccaatgtcctaacgtagtcgatctctacttcaaccttgctgctggtgaaggagtgtatttaccaaagctatgtgaatcacaagaagggaagtcaagaagagcaatgtcggaaattaggagctcgggaattgcaatggacactcttgcaccggttggaccagtcatgttgggcgagatagcacctgagccggctacttcaatggacaacatgccttacgtgccggcaccttcaccgtattaa

SEQ ID NO: 69 - DNA, 2006 bp, Arabidopsis thaliana
atgtcaaaggaagagccttgtgttacaagcaagactggatcgctggtctacgttctggtttcggaggttttacaggatttagcaccgacaacatatgttttcttcgcctctgcgcttcctgttattgcctttggcgagcaacttagccacgacacagagagatcgttgagcacagtggaaacgttagcatcaacagcgttatgtggagtgatacactcgttattgggaggacaaccattgttgatacttggagttgcagaaccaactgtcttaatgtacaaatacttgtacgacttcgctaaaggaagacctgaattgggcaaacaactctacttagcttgggttgcttgggtttgtgtgtggacggctttgttactattcctaatggcgatattcaacatggcttatatcatcaaccggttcacgaggatcgctggtgagctgtttggtatgttgatcgctgttctatttctccaacaaaccataaagggaatggtgagtgaatttaggattccaaaaggtgaagactcaaaacttgaaaagtatcagtttgagtggctctacacaaacggacttcttggccttattttcacagtcggtcttgtctacaccgctttgaagagcagaaaagcaaggtcttggccatacggaacaggatgttgccgaagcttcgttgcagactacggagttccgttgatggttgtggtttggacagcattgtctttcagtacgccatcaaaactaccctctggtgtcccgagaagactcgttagtcctcttccatgggactctgtttctttaacacattggactgtcatcaaggacatgggtaaagtctctcccggttacatatttgcagcgtttatacccgcattgatgatcgcaggcctctacttctttgaccacagcgttgtctcgcagctcgcgcagcagaaggagtttaacctcaagaacccttctgcatatcactacgacattctcttgttaggtttcatggtattgatctgtggaatgctcggtctaccgccttccaacggagtcctcccgcagtctcctatgcataccaaaagcctagctgttttcaaacgacagttaatgcggaggaagatggtgatgacagccaaagaaagcatcagacagaaagcaacgtcctctcaagtgtacgaggatatggaacaagtcttcatagaaatggacaaaagcccacttgctgagacacacacaacactgataaatgagctgcaagatctgaaagaggcagtgatgaagaagagtgacgacgacggggataccggcgaagagagtggtttcgatccagagaagcacgttgacgcttacttgcctgttcgagtcaacgagcagagagtgagcaacctgttgcaatcattgctagtgataggtgcagtgtttgctctaccggtcattaagctcataccgacttcacttctatggggatattttgcttacatggccattgatagcctcccagacaatcaattcttcgaacgaacagtacttctcttcgtcccaccaacccggagattcaaggtcttggaaggagcgcatgcatcgttcgtggagaaagttccgcataagtcaatcgctgcattcacgctatttcagatactctactttgggctttgctacggagtgacgtggattccagtggccggaatcatgtttccggttcttttcttccttttagtagccatcagacagtaccttctccctaagctctttaaaccagcctatctccgggaactcgatgcggcgggtatgaggagatccctggaactcctagaaacccgcttgaactgtctttcaggtcgaataactcggcgagaggggtccaagagtgtgatgctgagattctagacgagttaacaacgagcagaggcgagctcaaagtccgtacactcggtcataacgaagacaaaggccaccagatatatcccaaggagatagtagaagtaggggatggggacatgagttcttcgagagagtga